SEQUENCE LISTING (672 sequences)

SEQ ID NO: 1 - protein, 10 aa, Human immunodeficiency virus
KLTPLCVTLN

SEQ ID NO: 2 - protein, 9 aa, Human immunodeficiency virus
AEWDRVHPV

SEQ ID NO: 3 - protein, 9 aa, Human immunodeficiency virus
SLFNTVATL

SEQ ID NO: 4 - protein, 9 aa, Human immunodeficiency virus
ELHPDKWTV

SEQ ID NO: 5 - protein, 10 aa, Human immunodeficiency virus
GMDDPEREVL

SEQ ID NO: 6 - protein, 10 aa, Human immunodeficiency virus
GMDDPEKEVL

SEQ ID NO: 7 - protein, 10 aa, Human immunodeficiency virus
HLWRWGTMLL

SEQ ID NO: 8 - protein, 10 aa, Human immunodeficiency virus
LLLTRDGGVN

SEQ ID NO: 9 - protein, 10 aa, Human immunodeficiency virus
HLWKWSTMLL

SEQ ID NO: 10 - protein, 9 aa, Human immunodeficiency virus
ILKEPVHGV

SEQ ID NO: 11 - protein, 10 aa, Human immunodeficiency virus
KRWIILGLNK

SEQ ID NO: 12 - protein, 8 aa, Human immunodeficiency virus
CRIKQIIN

SEQ ID NO: 13 - protein, 10 aa, Human immunodeficiency virus
CRIKQIINMW

SEQ ID NO: 14 - protein, 10 aa, Human immunodeficiency virus
VSFEPIPIHF

SEQ ID NO: 15 - protein, 9 aa, Human immunodeficiency virus
RCSSNITGL

SEQ ID NO: 16 - protein, 10 aa, Human immunodeficiency virus
VSFEPIPIHY

SEQ ID NO: 17 - protein, 9 aa, Human immunodeficiency virus
CRIKQIVNM

SEQ ID NO: 18 - protein, 9 aa, Human immunodeficiency virus
IRSENITNN

SEQ ID NO: 19 - protein, 8 aa, Human immunodeficiency virus
IRIFIMIV

SEQ ID NO: 20 - protein, 10 aa, Human immunodeficiency virus
ISFDPIPIHY

SEQ ID NO: 21 - protein, 8 aa, Human immunodeficiency virus
YRTGDIIG

SEQ ID NO: 22 - protein, 10 aa, Human immunodeficiency virus
IRIGPGQTFY

SEQ ID NO: 23 - protein, 8 aa, Human immunodeficiency virus
GCSGKIIC

SEQ ID NO: 24 - protein, 8 aa, Human immunodeficiency virus
RRRAPQDS

SEQ ID NO: 25 - protein, 9 aa, Human immunodeficiency virus
IRSENITDN

SEQ ID NO: 26 - protein, 8 aa, Human immunodeficiency virus
CRIKQFIN

SEQ ID NO: 27 - protein, 9 aa, Human immunodeficiency virus
KRISIGPGR

SEQ ID NO: 28 - protein, 9 aa, Human immunodeficiency virus
GCQQIIEQL

SEQ ID NO: 29 - protein, 10 aa, Human immunodeficiency virus
GRRGWEILKY

SEQ ID NO: 30 - protein, 10 aa, Human immunodeficiency virus
SFEPIPIHYC

SEQ ID NO: 31 - protein, 10 aa, Human immunodeficiency virus
ELDKWASLWN

SEQ ID NO: 32 - protein, 10 aa, Human immunodeficiency virus
CTRPNNNTRK

SEQ ID NO: 33 - protein, 10 aa, Human immunodeficiency virus
GVAPTKAKRR

SEQ ID NO: 34 - protein, 10 aa, Human immunodeficiency virus
SFNCGGEFFY

SEQ ID NO: 35 - protein, 10 aa, Human immunodeficiency virus
ITLPCRIKQI

SEQ ID NO: 36 - protein, 10 aa, Human immunodeficiency virus
SSNITGLLLT

SEQ ID NO: 37 - protein, 10 aa, Human immunodeficiency virus
RLRPGGKKKY

SEQ ID NO: 38 - protein, 10 aa, Human immunodeficiency virus
AISPRTLNAW

SEQ ID NO: 39 - protein, 10 aa, Human immunodeficiency virus
AWEKIRLRPG

SEQ ID NO: 40 - protein, 10 aa, Human immunodeficiency virus
FRDYVDRFYK

SEQ ID NO: 41 - protein, 10 aa, Human immunodeficiency virus
LKEPVHGVYY

SEQ ID NO: 42 - protein, 10 aa, Human immunodeficiency virus
ETVPVKLKPG

SEQ ID NO: 43 - protein, 10 aa, Human immunodeficiency virus
ETPGIRYQYN

SEQ ID NO: 44 - protein, 10 aa, Human immunodeficiency virus
QKEPPFLWMG

SEQ ID NO: 45 - protein, 10 aa, Human immunodeficiency virus
NNETPGIRYQ

SEQ ID NO: 46 - protein, 10 aa, Human immunodeficiency virus
TPDKKHQKEP

SEQ ID NO: 47 - protein, 10 aa, Human immunodeficiency virus
IPHPAGLKKK

SEQ ID NO: 48 - protein, 10 aa, Human immunodeficiency virus
LVDFRELNKR

SEQ ID NO: 49 - protein, 10 aa, Human immunodeficiency virus
SAEPVPLQLP

SEQ ID NO: 50 - protein, 10 aa, Human immunodeficiency virus
  VARIANT (10): Wherein Xaa is any amino acid.
RGDPTGPKEX

SEQ ID NO: 51 - protein, 10 aa, Human immunodeficiency virus
LADQLIHLYY

SEQ ID NO: 52 - protein, 10 aa, Human immunodeficiency virus
QVDPGLADQL

SEQ ID NO: 53 - protein, 10 aa, Human immunodeficiency virus
LHSLGQHIYE

SEQ ID NO: 54 - protein, 10 aa, Human immunodeficiency virus
RAEDSGNESE

SEQ ID NO: 55 - protein, 10 aa, Human immunodeficiency virus
NLWVTVYYGV

SEQ ID NO: 56 - protein, 10 aa, Human immunodeficiency virus
GIKQLQARVL

SEQ ID NO: 57 - protein, 10 aa, Human immunodeficiency virus
QLQARVLAVE

SEQ ID NO: 58 - protein, 10 aa, Human immunodeficiency virus
KLTPLCVTLN

SEQ ID NO: 59 - protein, 10 aa, Human immunodeficiency virus
NMWQEVGKAM

SEQ ID NO: 60 - protein, 10 aa, Human immunodeficiency virus
QMHEDIISLW

SEQ ID NO: 61 - protein, 10 aa, Human immunodeficiency virus
DMRDNWRSEL

SEQ ID NO: 62 - protein, 10 aa, Human immunodeficiency virus
SLYNTVATLY

SEQ ID NO: 63 - protein, 10 aa, Human immunodeficiency virus
ELRSLYNTVA

SEQ ID NO: 64 - protein, 10 aa, Human immunodeficiency virus
EMMTACQGVG

SEQ ID NO: 65 - protein, 10 aa, Human immunodeficiency virus
DLNTMLNTVG

SEQ ID NO: 66 - protein, 10 aa, Human immunodeficiency virus
LLWKGEGAVV

SEQ ID NO: 67 - protein, 10 aa, Human immunodeficiency virus
ILKEPVHGVY

SEQ ID NO: 68 - protein, 10 aa, Human immunodeficiency virus
KLLWKGEGAV

SEQ ID NO: 69 - protein, 10 aa, Human immunodeficiency virus
HLKTAVQMAV

SEQ ID NO: 70 - protein, 10 aa, Human immunodeficiency virus
GLKKKKSVTV

SEQ ID NO: 71 - protein, 10 aa, Human immunodeficiency virus
ELHPDKWTVQ

SEQ ID NO: 72 - protein, 10 aa, Human immunodeficiency virus
KIEELRQHLL

SEQ ID NO: 73 - protein, 10 aa, Human immunodeficiency virus
KLLRGTKALT

SEQ ID NO: 74 - protein, 10 aa, Human immunodeficiency virus
QILVESPTVL

SEQ ID NO: 75 - protein, 10 aa, Human immunodeficiency virus
FLNKGLGISY

SEQ ID NO: 76 - protein, 10 aa, Human immunodeficiency virus
DLADQLIHLY

SEQ ID NO: 77 - protein, 10 aa, Human immunodeficiency virus
HIPLGDARLV

SEQ ID NO: 78 - protein, 10 aa, Human immunodeficiency virus
LLEELKNEAV

SEQ ID NO: 79 - protein, 10 aa, Human immunodeficiency virus
ILAIVVWTIV

SEQ ID NO: 80 - protein, 10 aa, Human immunodeficiency virus
HSFNCGGEFF

SEQ ID NO: 81 - protein, 10 aa, Human immunodeficiency virus
TLFCASDAKA

SEQ ID NO: 82 - protein, 10 aa, Human immunodeficiency virus
HSFNCRGEFF

SEQ ID NO: 83 - protein, 10 aa, Human immunodeficiency virus
SLWDQSLKPC

SEQ ID NO: 84 - protein, 10 aa, Human immunodeficiency virus
TVYYGVPVWK

SEQ ID NO: 85 - protein, 10 aa, Human immunodeficiency virus
VSFEPIPIHY

SEQ ID NO: 86 - protein, 10 aa, Human immunodeficiency virus
CTRPNNNTRK

SEQ ID NO: 87 - protein, 10 aa, Human immunodeficiency virus
SLYNTVATLY

SEQ ID NO: 88 - protein, 10 aa, Human immunodeficiency virus
LARNCRAPRK

SEQ ID NO: 89 - protein, 10 aa, Human immunodeficiency virus
RLRPGGKKKY

SEQ ID NO: 90 - protein, 10 aa, Human immunodeficiency virus
ILDIRQGPKE

SEQ ID NO: 91 - protein, 10 aa, Human immunodeficiency virus
LVDFRELNKR

SEQ ID NO: 92 - protein, 10 aa, Human immunodeficiency virus
QLDCTHLEGK

SEQ ID NO: 93 - protein, 10 aa, Human immunodeficiency virus
AVFIHNFKRK

SEQ ID NO: 94 - protein, 10 aa, Human immunodeficiency virus
QIIEQLIKKE

SEQ ID NO: 95 - protein, 10 aa, Human immunodeficiency virus
GIPHPAGLKK

SEQ ID NO: 96 - protein, 10 aa, Human immunodeficiency virus
KVYLAWVPAH

SEQ ID NO: 97 - protein, 10 aa, Human immunodeficiency virus
AIFQSSMTKI

SEQ ID NO: 98 - protein, 10 aa, Human immunodeficiency virus
KLVDFRELNK

SEQ ID NO: 99 - protein, 10 aa, Human immunodeficiency virus
KILYQSNPYP

SEQ ID NO: 100 - protein, 10 aa, Human immunodeficiency virus
TACNNCYCKK

SEQ ID NO: 101 - protein, 10 aa, Human immunodeficiency virus
ALTALITPKK

SEQ ID NO: 102 - protein, 10 aa, Human immunodeficiency virus
KLTEDRWNKP

SEQ ID NO: 103 - protein, 10 aa, Human immunodeficiency virus
WTLELLEELK

SEQ ID NO: 104 - protein, 10 aa, Human immunodeficiency virus
RLIDRIRERA

SEQ ID NO: 105 - protein, 10 aa, Human immunodeficiency virus
TVQCTHGIKP

SEQ ID NO: 106 - protein, 10 aa, Human immunodeficiency virus
FAILKCNDKK

SEQ ID NO: 107 - protein, 10 aa, Human immunodeficiency virus
NVTENFNMWK

SEQ ID NO: 108 - protein, 10 aa, Human immunodeficiency virus
TITLPCRIKQ

SEQ ID NO: 109 - protein, 10 aa, Human immunodeficiency virus
TVYYGVPVWK

SEQ ID NO: 110 - protein, 10 aa, Human immunodeficiency virus
CTRPNNNTRK

SEQ ID NO: 111 - protein, 10 aa, Human immunodeficiency virus
VTENFNMWKN

SEQ ID NO: 112 - protein, 10 aa, Human immunodeficiency virus
IRLRPGGKKK

SEQ ID NO: 113 - protein, 10 aa, Human immunodeficiency virus
KIRLRPGGKK

SEQ ID NO: 114 - protein, 10 aa, Human immunodeficiency virus
LVQNANPDCK

SEQ ID NO: 115 - protein, 10 aa, Human immunodeficiency virus
ARNCRAPRKK

SEQ ID NO: 116 - protein, 10 aa, Human immunodeficiency virus
FTTPDKKHQK

SEQ ID NO: 117 - protein, 10 aa, Human immunodeficiency virus
GIPHPAGLKK

SEQ ID NO: 118 - protein, 10 aa, Human immunodeficiency virus
TTPDKKHQKE

SEQ ID NO: 119 - protein, 10 aa, Human immunodeficiency virus
IPHPAGLKKK

SEQ ID NO: 120 - protein, 10 aa, Human immunodeficiency virus
AVFIHNFKRK

SEQ ID NO: 121 - protein, 10 aa, Human immunodeficiency virus
NTPVFAIKKK

SEQ ID NO: 122 - protein, 10 aa, Human immunodeficiency virus
PGMDGPKVKQ

SEQ ID NO: 123 - protein, 10 aa, Human immunodeficiency virus
QVRDQAEHLK

SEQ ID NO: 124 - protein, 10 aa, Human immunodeficiency virus
PTVLESGTKE

SEQ ID NO: 125 - protein, 10 aa, Human immunodeficiency virus
TACNNCYCKK

SEQ ID NO: 126 - protein, 10 aa, Human immunodeficiency virus
IKPPLPSVKK

SEQ ID NO: 127 - protein, 10 aa, Human immunodeficiency virus
ALTALITPKK

SEQ ID NO: 128 - protein, 10 aa, Human immunodeficiency virus
WTLELLEELK

SEQ ID NO: 129 - protein, 10 aa, Human immunodeficiency virus
WTIVFIEYRK

SEQ ID NO: 130 - protein, 10 aa, Human immunodeficiency virus
RYLKDQQLLG

SEQ ID NO: 131 - protein, 10 aa, Human immunodeficiency virus
SYHRLRDLLL

SEQ ID NO: 132 - protein, 10 aa, Human immunodeficiency virus
IYQEPFKNLK

SEQ ID NO: 133 - protein, 10 aa, Human immunodeficiency virus
VYYDPSKDLI

SEQ ID NO: 134 - protein, 10 aa, Human immunodeficiency virus
YYFDCFSESA

SEQ ID NO: 135 - protein, 10 aa, Human immunodeficiency virus
PYNEWTLELL

SEQ ID NO: 136 - protein, 10 aa, Human immunodeficiency virus
MIVGGLIGLR

SEQ ID NO: 137 - protein, 10 aa, Human immunodeficiency virus
SLAEEEIIIR

SEQ ID NO: 138 - protein, 10 aa, Human immunodeficiency virus
IVQQQNNLLR

SEQ ID NO: 139 - protein, 10 aa, Human immunodeficiency virus
IVQQQSNLLR

SEQ ID NO: 140 - protein, 10 aa, Human immunodeficiency virus
SLAEEEVVIR

SEQ ID NO: 141 - protein, 10 aa, Human immunodeficiency virus
STVQCTHGIR

SEQ ID NO: 142 - protein, 10 aa, Human immunodeficiency virus
LQARVLAVER

SEQ ID NO: 143 - protein, 10 aa, Human immunodeficiency virus
LVWASRELER

SEQ ID NO: 144 - protein, 10 aa, Human immunodeficiency virus
IVWASRELER

SEQ ID NO: 145 - protein, 10 aa, Human immunodeficiency virus
IILGLNKIVR

SEQ ID NO: 146 - protein, 10 aa, Human immunodeficiency virus
QMVHQAISPR

SEQ ID NO: 147 - protein, 10 aa, Human immunodeficiency virus
KIQNFRVYYR

SEQ ID NO: 148 - protein, 10 aa, Human immunodeficiency virus
LVDFRELNKR

SEQ ID NO: 149 - protein, 10 aa, Human immunodeficiency virus
KLVDFRELNK

SEQ ID NO: 150 - protein, 10 aa, Human immunodeficiency virus
SMTKILEPFR

SEQ ID NO: 151 - protein, 10 aa, Human immunodeficiency virus
SINNETPGIR

SEQ ID NO: 152 - protein, 10 aa, Human immunodeficiency virus
GIGGYSAGER

SEQ ID NO: 153 - protein, 10 aa, Human immunodeficiency virus
TFYVDGAANR

SEQ ID NO: 154 - protein, 10 aa, Human immunodeficiency virus
SQIIEQLIKK

SEQ ID NO: 155 - protein, 10 aa, Human immunodeficiency virus
GTRQARRNRR

SEQ ID NO: 156 - protein, 10 aa, Human immunodeficiency virus
KTACTNCYCK

SEQ ID NO: 157 - protein, 10 aa, Human immunodeficiency virus
AILGHIVSPR

SEQ ID NO: 158 - protein, 10 aa, Human immunodeficiency virus
QVMIVWQVDR

SEQ ID NO: 159 - protein, 10 aa, Human immunodeficiency virus
LQQLLFIHFR

SEQ ID NO: 160 - protein, 10 aa, Human immunodeficiency virus
KILRQRKIDR

SEQ ID NO: 161 - protein, 10 aa, Human immunodeficiency virus
EITTHSFNCR

SEQ ID NO: 162 - protein, 10 aa, Human immunodeficiency virus
IVQQQNNLLR

SEQ ID NO: 163 - protein, 10 aa, Human immunodeficiency virus
MIVGGLIGLR

SEQ ID NO: 164 - protein, 10 aa, Human immunodeficiency virus
ASITLTVQAR

SEQ ID NO: 165 - protein, 10 aa, Human immunodeficiency virus
AIAVAEGTDR

SEQ ID NO: 166 - protein, 10 aa, Human immunodeficiency virus
IVQQQSNLLR

SEQ ID NO: 167 - protein, 10 aa, Human immunodeficiency virus
AVLSIVNRVR

SEQ ID NO: 168 - protein, 10 aa, Human immunodeficiency virus
IILGLNKIVR

SEQ ID NO: 169 - protein, 10 aa, Human immunodeficiency virus
GVGGPGHKAR

SEQ ID NO: 170 - protein, 10 aa, Human immunodeficiency virus
YVDRFYKTLR

SEQ ID NO: 171 - protein, 10 aa, Human immunodeficiency virus
YSPVSILDIR

SEQ ID NO: 172 - protein, 10 aa, Human immunodeficiency virus
ELKKIIGQVR

SEQ ID NO: 173 - protein, 10 aa, Human immunodeficiency virus
LVDFRELNKR

SEQ ID NO: 174 - protein, 10 aa, Human immunodeficiency virus
GSDLEIGQHR

SEQ ID NO: 175 - protein, 10 aa, Human immunodeficiency virus
SMTKILEPFR

SEQ ID NO: 176 - protein, 10 aa, Human immunodeficiency virus
SINNETPGIR

SEQ ID NO: 177 - protein, 10 aa, Human immunodeficiency virus
GIGGYSAGER

SEQ ID NO: 178 - protein, 10 aa, Human immunodeficiency virus
EAELELAENR

SEQ ID NO: 179 - protein, 10 aa, Human immunodeficiency virus
KIQNFRVYYR

SEQ ID NO: 180 - protein, 10 aa, Human immunodeficiency virus
EGTRQARRNR

SEQ ID NO: 181 - protein, 10 aa, Human immunodeficiency virus
GISYGRKKRR

SEQ ID NO: 182 - protein, 10 aa, Human immunodeficiency virus
EVHIPLGDAR

SEQ ID NO: 183 - protein, 10 aa, Human immunodeficiency virus
QVMIVWQVDR

SEQ ID NO: 184 - protein, 10 aa, Human immunodeficiency virus
HSRIGITRQR

SEQ ID NO: 185 - protein, 10 aa, Human immunodeficiency virus
DSGNESEGDR

SEQ ID NO: 186 - protein, 10 aa, Human immunodeficiency virus
GVAPTKAKRR

SEQ ID NO: 187 - protein, 10 aa, Human immunodeficiency virus
AVLSIVNRVR

SEQ ID NO: 188 - protein, 10 aa, Human immunodeficiency virus
IVQQQNNLLR

SEQ ID NO: 189 - protein, 10 aa, Human immunodeficiency virus
IVQQQSNLLR

SEQ ID NO: 190 - protein, 10 aa, Human immunodeficiency virus
TVYYGVPVWK

SEQ ID NO: 191 - protein, 10 aa, Human immunodeficiency virus
NVTENFNMWK

SEQ ID NO: 192 - protein, 10 aa, Human immunodeficiency virus
STVQCTHGIR

SEQ ID NO: 193 - protein, 10 aa, Human immunodeficiency virus
GVGGPGHKAR

SEQ ID NO: 194 - protein, 10 aa, Human immunodeficiency virus
GVGGPSHKAR

SEQ ID NO: 195 - protein, 10 aa, Human immunodeficiency virus
LVWASRELER

SEQ ID NO: 196 - protein, 10 aa, Human immunodeficiency virus
IVWASRELER

SEQ ID NO: 197 - protein, 10 aa, Human immunodeficiency virus
AVFIHNFKRK

SEQ ID NO: 198 - protein, 10 aa, Human immunodeficiency virus
LVDFRELNKR

SEQ ID NO: 199 - protein, 10 aa, Human immunodeficiency virus
LVEICTEMEK

SEQ ID NO: 200 - protein, 10 aa, Human immunodeficiency virus
QVRDQAEHLK

SEQ ID NO: 201 - protein, 10 aa, Human immunodeficiency virus
LVKLWYQLEK

SEQ ID NO: 202 - protein, 10 aa, Human immunodeficiency virus
FTTPDKKHQK

SEQ ID NO: 203 - protein, 10 aa, Human immunodeficiency virus
DSWTVNDIQK

SEQ ID NO: 204 - protein, 10 aa, Human immunodeficiency virus
NTPVFAIKKK

SEQ ID NO: 205 - protein, 10 aa, Human immunodeficiency virus
GTRQARRNRR

SEQ ID NO: 206 - protein, 10 aa, Human immunodeficiency virus
KTACTNCYCK

SEQ ID NO: 207 - protein, 10 aa, Human immunodeficiency virus
EVHIPLGDAR

SEQ ID NO: 208 - protein, 10 aa, Human immunodeficiency virus
QVMIVWQVDR

SEQ ID NO: 209 - protein, 10 aa, Human immunodeficiency virus
WTLELLEELK

SEQ ID NO: 210 - protein, 10 aa, Human immunodeficiency virus
DSGNESEGDR

SEQ ID NO: 211 - protein, 10 aa, Human immunodeficiency virus
KPVVSTQLLL

SEQ ID NO: 212 - protein, 10 aa, Human immunodeficiency virus
RPVVSTQLLL

SEQ ID NO: 213 - protein, 10 aa, Human immunodeficiency virus
KPCVKLTPLC

SEQ ID NO: 214 - protein, 10 aa, Human immunodeficiency virus
RCSSNITGLL

SEQ ID NO: 215 - protein, 10 aa, Human immunodeficiency virus
APTKAKRRVV

SEQ ID NO: 216 - protein, 10 aa, Human immunodeficiency virus
RAIEAQQHLL

SEQ ID NO: 217 - protein, 10 aa, Human immunodeficiency virus
GPCKNVSTVQ

SEQ ID NO: 218 - protein, 10 aa, Human immunodeficiency virus
TPQDLNTMLN

SEQ ID NO: 219 - protein, 10 aa, Human immunodeficiency virus
TPQDLNMMLN

SEQ ID NO: 220 - protein, 10 aa, Human immunodeficiency virus
GPGHKARVLA

SEQ ID NO: 221 - protein, 10 aa, Human immunodeficiency virus
APRKKGCWKC

SEQ ID NO: 222 - protein, 10 aa, Human immunodeficiency virus
QPDKSESELV

SEQ ID NO: 223 - protein, 10 aa, Human immunodeficiency virus
GPKVKQWPLT

SEQ ID NO: 224 - protein, 10 aa, Human immunodeficiency virus
SPAIFQSSMT

SEQ ID NO: 225 - protein, 10 aa, Human immunodeficiency virus
SPIETVPVKL

SEQ ID NO: 226 - protein, 10 aa, Human immunodeficiency virus
KIEELRQHLL

SEQ ID NO: 227 - protein, 10 aa, Human immunodeficiency virus
QVRDQAEHLK

SEQ ID NO: 228 - protein, 10 aa, Human immunodeficiency virus
LVSQIIEQLI

SEQ ID NO: 229 - protein, 10 aa, Human immunodeficiency virus
IPAETGQETA

SEQ ID NO: 230 - protein, 10 aa, Human immunodeficiency virus
LPPLERLTLD

SEQ ID NO: 231 - protein, 10 aa, Human immunodeficiency virus
  VARIANT (5): Wherein Xaa is any amino acid.
GPKEXKKKVE

SEQ ID NO: 232 - protein, 10 aa, Human immunodeficiency virus
KPPLPSVTKL

SEQ ID NO: 233 - protein, 10 aa, Human immunodeficiency virus
KPPLPSVKKL

SEQ ID NO: 234 - protein, 10 aa, Human immunodeficiency virus
FPRIWLHSLG

SEQ ID NO: 235 - protein, 10 aa, Human immunodeficiency virus
LVILAIVALV

SEQ ID NO: 236 - protein, 10 aa, Human immunodeficiency virus
NAKTIIVQLN

SEQ ID NO: 237 - protein, 10 aa, Human immunodeficiency virus
PTKAKRRVVQ

SEQ ID NO: 238 - protein, 10 aa, Human immunodeficiency virus
LYKYKVVKIE

SEQ ID NO: 239 - protein, 10 aa, Human immunodeficiency virus
TLPCRIKQII

SEQ ID NO: 240 - protein, 10 aa, Human immunodeficiency virus
VPVWKEATTT

SEQ ID NO: 241 - protein, 10 aa, Human immunodeficiency virus
VWGIKQLQAR

SEQ ID NO: 242 - protein, 10 aa, Human immunodeficiency virus
DAKAYDTEVH

SEQ ID NO: 243 - protein, 10 aa, Human immunodeficiency virus
FNCGKEGHLA

SEQ ID NO: 244 - protein, 10 aa, Human immunodeficiency virus
NAWVKVVEEK

SEQ ID NO: 245 - protein, 10 aa, Human immunodeficiency virus
DCKTILKALG

SEQ ID NO: 246 - protein, 10 aa, Human immunodeficiency virus
NAWVKVIEEK

SEQ ID NO: 247 - protein, 10 aa, Human immunodeficiency virus
GLKKKKSVTV

SEQ ID NO: 248 - protein, 10 aa, Human immunodeficiency virus
GPKVKQWPLT

SEQ ID NO: 249 - protein, 10 aa, Human immunodeficiency virus
AIKKKDSTKW

SEQ ID NO: 250 - protein, 10 aa, Human immunodeficiency virus
FAIKKKDSTK

SEQ ID NO: 251 - protein, 10 aa, Human immunodeficiency virus
QHRTKIEELR

SEQ ID NO: 252 - protein, 10 aa, Human immunodeficiency virus
ELKKIIGQVR

SEQ ID NO: 253 - protein, 10 aa, Human immunodeficiency virus
AGLKKKKSVT

SEQ ID NO: 254 - protein, 10 aa, Human immunodeficiency virus
GIKVKQLCKL

SEQ ID NO: 255 - protein, 10 aa, Human immunodeficiency virus
IIKILYQSNP

SEQ ID NO: 256 - protein, 10 aa, Human immunodeficiency virus
ESKKKVERET

SEQ ID NO: 257 - protein, 10 aa, Human immunodeficiency virus
TPKKIKPPLP

SEQ ID NO: 258 - protein, 10 aa, Human immunodeficiency virus
AGHNKVGSLQ

SEQ ID NO: 259 - protein, 10 aa, Human immunodeficiency virus
EAIIRILQQL

SEQ ID NO: 260 - protein, 10 aa, Human immunodeficiency virus
WLIDRIRERA

SEQ ID NO: 261 - protein, 10 aa, Human immunodeficiency virus
ERYLKDQQLL

SEQ ID NO: 262 - protein, 10 aa, Human immunodeficiency virus
FSYHRLRDLL

SEQ ID NO: 263 - protein, 10 aa, Human immunodeficiency virus
EAQQHLLQLT

SEQ ID NO: 264 - protein, 10 aa, Human immunodeficiency virus
MRDNWRSELY

SEQ ID NO: 265 - protein, 10 aa, Human immunodeficiency virus
CRIKQIVNMW

SEQ ID NO: 266 - protein, 10 aa, Human immunodeficiency virus
PTKAKRRVVQ

SEQ ID NO: 267 - protein, 10 aa, Human immunodeficiency virus
TLPCRIKQII

SEQ ID NO: 268 - protein, 10 aa, Human immunodeficiency virus
DRFFKTLRAE

SEQ ID NO: 269 - protein, 10 aa, Human immunodeficiency virus
DRFYKTLRAE

SEQ ID NO: 270 - protein, 10 aa, Human immunodeficiency virus
ERFAVNPGLL

SEQ ID NO: 271 - protein, 10 aa, Human immunodeficiency virus
SLYNTVATLY

SEQ ID NO: 272 - protein, 10 aa, Human immunodeficiency virus
GAANRETKLG

SEQ ID NO: 273 - protein, 10 aa, Human immunodeficiency virus
NRETKLGKAG

SEQ ID NO: 274 - protein, 10 aa, Human immunodeficiency virus
KLVGKLNWAS

SEQ ID NO: 275 - protein, 10 aa, Human immunodeficiency virus
EPFRKQNPDI

SEQ ID NO: 276 - protein, 10 aa, Human immunodeficiency virus
LTEEKIKALV

SEQ ID NO: 277 - protein, 10 aa, Human immunodeficiency virus
WTVNDIQKLV

SEQ ID NO: 278 - protein, 10 aa, Human immunodeficiency virus
TRQARRNRRR

SEQ ID NO: 279 - protein, 10 aa, Human immunodeficiency virus
GRKKRRQRRR

SEQ ID NO: 280 - protein, 10 aa, Human immunodeficiency virus
DRWNKPQKTK

SEQ ID NO: 281 - protein, 10 aa, Human immunodeficiency virus
ERDWHLGQGV

SEQ ID NO: 282 - protein, 10 aa, Human immunodeficiency virus
QREPHNEWTL

SEQ ID NO: 283 - protein, 10 aa, Human immunodeficiency virus
LRQRKIDRLI

SEQ ID NO: 284 - protein, 10 aa, Human immunodeficiency virus
DLRSLCLFSY

SEQ ID NO: 285 - protein, 10 aa, Human immunodeficiency virus
QQHLLQLTVW

SEQ ID NO: 286 - protein, 10 aa, Human immunodeficiency virus
RLRPGGKKKY

SEQ ID NO: 287 - protein, 10 aa, Human immunodeficiency virus
SLYNTVATLY

SEQ ID NO: 288 - protein, 10 aa, Human immunodeficiency virus
DIRQGPKEPF

SEQ ID NO: 289 - protein, 10 aa, Human immunodeficiency virus
RQANFLGKIW

SEQ ID NO: 290 - protein, 10 aa, Human immunodeficiency virus
ILKEPVHGVY

SEQ ID NO: 291 - protein, 10 aa, Human immunodeficiency virus
GQGQWTYQIY

SEQ ID NO: 292 - protein, 10 aa, Human immunodeficiency virus
VQMAVFIHNF

SEQ ID NO: 293 - protein, 10 aa, Human immunodeficiency virus
IQKLVGKLNW

SEQ ID NO: 294 - protein, 10 aa, Human immunodeficiency virus
EQLIKKEKVY

SEQ ID NO: 295 - protein, 10 aa, Human immunodeficiency virus
YQYNVLPQGW

SEQ ID NO: 296 - protein, 10 aa, Human immunodeficiency virus
HQKEPPFLWM

SEQ ID NO: 297 - protein, 10 aa, Human immunodeficiency virus
LLKTVRLIKF

SEQ ID NO: 298 - protein, 10 aa, Human immunodeficiency virus
FLNKGLGISY

SEQ ID NO: 299 - protein, 10 aa, Human immunodeficiency virus
DLADQLIHLY

SEQ ID NO: 300 - protein, 10 aa, Human immunodeficiency virus
HLGQGVSIEW

SEQ ID NO: 301 - protein, 10 aa, Human immunodeficiency virus
ILQQLLFIHF

SEQ ID NO: 302 - protein, 10 aa, Human immunodeficiency virus
CRIKQIINMW

SEQ ID NO: 303 - protein, 10 aa, Human immunodeficiency virus
CRIKQIVNMW

SEQ ID NO: 304 - protein, 10 aa, Human immunodeficiency virus
RRVVQREKRA

SEQ ID NO: 305 - protein, 10 aa, Human immunodeficiency virus
KRRVVQREKR

SEQ ID NO: 306 - protein, 10 aa, Human immunodeficiency virus
RRVVEREKRA

SEQ ID NO: 307 - protein, 10 aa, Human immunodeficiency virus
IRSENLTNNA

SEQ ID NO: 308 - protein, 10 aa, Human immunodeficiency virus
KRRVVEREKR

SEQ ID NO: 309 - protein, 10 aa, Human immunodeficiency virus
KRWIILGLNK

SEQ ID NO: 310 - protein, 10 aa, Human immunodeficiency virus
IRQGPKEPFR

SEQ ID NO: 311 - protein, 10 aa, Human immunodeficiency virus
IRLRPGGKKK

SEQ ID NO: 312 - protein, 10 aa, Human immunodeficiency virus
ARNCRAPRKK

SEQ ID NO: 313 - protein, 10 aa, Human immunodeficiency virus
KRKGGIGGYS

SEQ ID NO: 314 - protein, 10 aa, Human immunodeficiency virus
KRTQDFWEVQ

SEQ ID NO: 315 - protein, 10 aa, Human immunodeficiency virus
HRTKIEELRQ

SEQ ID NO: 316 - protein, 10 aa, Human immunodeficiency virus
KQNPDIVIYQ

SEQ ID NO: 317 - protein, 10 aa, Human immunodeficiency virus
VRDQAEHLKT

SEQ ID NO: 318 - protein, 10 aa, Human immunodeficiency virus
IRYQYNVLPQ

SEQ ID NO: 319 - protein, 10 aa, Human immunodeficiency virus
KALTEVIPLT

SEQ ID NO: 320 - protein, 10 aa, Human immunodeficiency virus
WGFTTPDKKH

SEQ ID NO: 321 - protein, 10 aa, Human immunodeficiency virus
GRSAEPVPLQ

SEQ ID NO: 322 - protein, 10 aa, Human immunodeficiency virus
RRAPQDSQTH

SEQ ID NO: 323 - protein, 10 aa, Human immunodeficiency virus
NRWQVMIVWQ

SEQ ID NO: 324 - protein, 10 aa, Human immunodeficiency virus
ARLVITTYWG

SEQ ID NO: 325 - protein, 10 aa, Human immunodeficiency virus
SRIGIIQQRR

SEQ ID NO: 326 - protein, 10 aa, Human immunodeficiency virus
LRQRKIDRLI

SEQ ID NO: 327 - protein, 10 aa, Human immunodeficiency virus
KPCVKLTPLC

SEQ ID NO: 328 - protein, 10 aa, Human immunodeficiency virus
KPVVSTQLLL

SEQ ID NO: 329 - protein, 10 aa, Human immunodeficiency virus
RPVVSTQLLL

SEQ ID NO: 330 - protein, 10 aa, Human immunodeficiency virus
CPKVSFEPIP

SEQ ID NO: 331 - protein, 10 aa, Human immunodeficiency virus
RAIEAQQHLL

SEQ ID NO: 332 - protein, 10 aa, Human immunodeficiency virus
NAKTIIVQLN

SEQ ID NO: 333 - protein, 10 aa, Human immunodeficiency virus
LPCRIKQIIN

SEQ ID NO: 334 - protein, 10 aa, Human immunodeficiency virus
GPKEPFRDYV

SEQ ID NO: 335 - protein, 10 aa, Human immunodeficiency virus
GPAATLEEMM

SEQ ID NO: 336 - protein, 10 aa, Human immunodeficiency virus
GPGATLEEMM

SEQ ID NO: 337 - protein, 10 aa, Human immunodeficiency virus
TPQDLNTMLN

SEQ ID NO: 338 - protein, 10 aa, Human immunodeficiency virus
GPKVKQWPLT

SEQ ID NO: 339 - protein, 10 aa, Human immunodeficiency virus
VPVKLKPGMD

SEQ ID NO: 340 - protein, 10 aa, Human immunodeficiency virus
EPPFLWMGYE

SEQ ID NO: 341 - protein, 10 aa, Human immunodeficiency virus
TPPLVKLWYQ

SEQ ID NO: 342 - protein, 10 aa, Human immunodeficiency virus
SPAIFQSSMT

SEQ ID NO: 343 - protein, 10 aa, Human immunodeficiency virus
EPIVGAETFY

SEQ ID NO: 344 - protein, 10 aa, Human immunodeficiency virus
NPDIVIYQYM

SEQ ID NO: 345 - protein, 10 aa, Human immunodeficiency virus
KPGMDGPKVK

SEQ ID NO: 346 - protein, 10 aa, Human immunodeficiency virus
LPPLERLTLD

SEQ ID NO: 347 - protein, 10 aa, Human immunodeficiency virus
GPKESKKKVE

SEQ ID NO: 348 - protein, 10 aa, Human immunodeficiency virus
TPKKIKPPLP

SEQ ID NO: 349 - protein, 10 aa, Human immunodeficiency virus
KSLVKHHMYI

SEQ ID NO: 350 - protein, 10 aa, Human immunodeficiency virus
FPRIWLHSLG

SEQ ID NO: 351 - protein, 10 aa, Human immunodeficiency virus
QPLVILAIVA

SEQ ID NO: 352 - protein, 10 aa, Human immunodeficiency virus
IHYCAPAGFA

SEQ ID NO: 353 - protein, 10 aa, Human immunodeficiency virus
MHEDIISLWD

SEQ ID NO: 354 - protein, 10 aa, Human immunodeficiency virus
YHRLRDLLLI

SEQ ID NO: 355 - protein, 10 aa, Human immunodeficiency virus
QHLLQLTVWG

SEQ ID NO: 356 - protein, 10 aa, Human immunodeficiency virus
THGIKPVVST

SEQ ID NO: 357 - protein, 10 aa, Human immunodeficiency virus
THGIRPVVST

SEQ ID NO: 358 - protein, 10 aa, Human immunodeficiency virus
VHNVWATHAC

SEQ ID NO: 359 - protein, 10 aa, Human immunodeficiency virus
GHQAAMQMLK

SEQ ID NO: 360 - protein, 10 aa, Human immunodeficiency virus
SHKGRPGNFL

SEQ ID NO: 361 - protein, 10 aa, Human immunodeficiency virus
LHPVHAGPIA

SEQ ID NO: 362 - protein, 10 aa, Human immunodeficiency virus
VHQAISPRTL

SEQ ID NO: 363 - protein, 10 aa, Human immunodeficiency virus
AHTNDVKQLT

SEQ ID NO: 364 - protein, 10 aa, Human immunodeficiency virus
KHQKEPPFLW

SEQ ID NO: 365 - protein, 10 aa, Human immunodeficiency virus
QHRTKIEELR

SEQ ID NO: 366 - protein, 10 aa, Human immunodeficiency virus
EHLKTAVQMA

SEQ ID NO: 367 - protein, 10 aa, Human immunodeficiency virus
KIEELRQHLL

SEQ ID NO: 368 - protein, 10 aa, Human immunodeficiency virus
QPDKSESELV

SEQ ID NO: 369 - protein, 10 aa, Human immunodeficiency virus
LTEEAELELA

SEQ ID NO: 370 - protein, 10 aa, Human immunodeficiency virus
LTEEKIKALV

SEQ ID NO: 371 - protein, 10 aa, Human immunodeficiency virus
SAEPVPLQLP

SEQ ID NO: 372 - protein, 10 aa, Human immunodeficiency virus
KHPGSQPKTA

SEQ ID NO: 373 - protein, 10 aa, Human immunodeficiency virus
IHLYYFDCFS

SEQ ID NO: 374 - protein, 10 aa, Human immunodeficiency virus
IHLHYFDCFS

SEQ ID NO: 375 - protein, 10 aa, Human immunodeficiency virus
PHNEWTLELL

SEQ ID NO: 376 - protein, 10 aa, Human immunodeficiency virus
ESEGDQEELS

SEQ ID NO: 377 - protein, 10 aa, Human immunodeficiency virus
MHEDIISLWD

SEQ ID NO: 378 - protein, 10 aa, Human immunodeficiency virus
MRDNWRSELY

SEQ ID NO: 379 - protein, 10 aa, Human immunodeficiency virus
CRIKQIINMW

SEQ ID NO: 380 - protein, 10 aa, Human immunodeficiency virus
IRPVVSTQLL

SEQ ID NO: 381 - protein, 10 aa, Human immunodeficiency virus
CRIKQIVNMW

SEQ ID NO: 382 - protein, 10 aa, Human immunodeficiency virus
ERYLKDQQLL

SEQ ID NO: 383 - protein, 10 aa, Human immunodeficiency virus
YHRLRDLLLI

SEQ ID NO: 384 - protein, 10 aa, Human immunodeficiency virus
GHQAAMQMLK

SEQ ID NO: 385 - protein, 10 aa, Human immunodeficiency virus
LHPVHAGPIA

SEQ ID NO: 386 - protein, 10 aa, Human immunodeficiency virus
ERFAVNPGLL

SEQ ID NO: 387 - protein, 10 aa, Human immunodeficiency virus
SRELERFALN

SEQ ID NO: 388 - protein, 10 aa, Human immunodeficiency virus
AHTNDVKQLT

SEQ ID NO: 389 - protein, 10 aa, Human immunodeficiency virus
KHQKEPPFLW

SEQ ID NO: 390 - protein, 10 aa, Human immunodeficiency virus
EHLKTAVQMA

SEQ ID NO: 391 - protein, 10 aa, Human immunodeficiency virus
QHRTKIEELR

SEQ ID NO: 392 - protein, 10 aa, Human immunodeficiency virus
LTEEKIKALV

SEQ ID NO: 393 - protein, 10 aa, Human immunodeficiency virus
VYYDPSKDLI

SEQ ID NO: 394 - protein, 10 aa, Human immunodeficiency virus
WTVNDIQKLV

SEQ ID NO: 395 - protein, 10 aa, Human immunodeficiency virus
GGNEQVDKLV

SEQ ID NO: 396 - protein, 10 aa, Human immunodeficiency virus
GRSAEPVPLQ

SEQ ID NO: 397 - protein, 10 aa, Human immunodeficiency virus
ERETETDPVH

SEQ ID NO: 398 - protein, 10 aa, Human immunodeficiency virus
WHLGQGVSIE

SEQ ID NO: 399 - protein, 10 aa, Human immunodeficiency virus
THPRISSEVH

SEQ ID NO: 400 - protein, 10 aa, Human immunodeficiency virus
WTLELLEELK

SEQ ID NO: 401 - protein, 10 aa, Human immunodeficiency virus
LRQRKIDRLI

SEQ ID NO: 402 - protein, 10 aa, Human immunodeficiency virus
QEVGKAMYAP

SEQ ID NO: 403 - protein, 10 aa, Human immunodeficiency virus
VELLGRRGWE

SEQ ID NO: 404 - protein, 10 aa, Human immunodeficiency virus
LELDKWASLW

SEQ ID NO: 405 - protein, 10 aa, Human immunodeficiency virus
GEFFYCNTSG

SEQ ID NO: 406 - protein, 10 aa, Human immunodeficiency virus
TEVHNVWATH

SEQ ID NO: 407 - protein, 10 aa, Human immunodeficiency virus
SELYKYKVVK

SEQ ID NO: 408 - protein, 10 aa, Human immunodeficiency virus
KEATTTLFCA

SEQ ID NO: 409 - protein, 10 aa, Human immunodeficiency virus
IEVKDTKEAL

SEQ ID NO: 410 - protein, 10 aa, Human immunodeficiency virus
EEAAEWDRLH

SEQ ID NO: 411 - protein, 10 aa, Human immunodeficiency virus
GEIYKRWIIL

SEQ ID NO: 412 - protein, 10 aa, Human immunodeficiency virus
REPRGSDIAG

SEQ ID NO: 413 - protein, 10 aa, Human immunodeficiency virus
WEFVNTPPLV

SEQ ID NO: 414 - protein, 10 aa, Human immunodeficiency virus
AETFYVDGAA

SEQ ID NO: 415 - protein, 10 aa, Human immunodeficiency virus
TELQAIHLAL

SEQ ID NO: 416 - protein, 10 aa, Human immunodeficiency virus
LEVNIVTDSQ

SEQ ID NO: 417 - protein, 10 aa, Human immunodeficiency virus
YELHPDKWTV

SEQ ID NO: 418 - protein, 10 aa, Human immunodeficiency virus
NDVKQLTEAV

SEQ ID NO: 419 - protein, 10 aa, Human immunodeficiency virus
TEEAELELAE

SEQ ID NO: 420 - protein, 10 aa, Human immunodeficiency virus
GDAYFSVPLD

SEQ ID NO: 421 - protein, 10 aa, Human immunodeficiency virus
EELLKTVRLI

SEQ ID NO: 422 - protein, 10 aa, Human immunodeficiency virus
LEPWKHPGSQ

SEQ ID NO: 423 - protein, 10 aa, Human immunodeficiency virus
IEWRKKRYST

SEQ ID NO: 424 - protein, 10 aa, Human immunodeficiency virus
IEWRKRRYST

SEQ ID NO: 425 - protein, 10 aa, Human immunodeficiency virus
YETYGDTWAG

SEQ ID NO: 426 - protein, 10 aa, Human immunodeficiency virus
VEMGHHAPWD

SEQ ID NO: 427 - protein, 10 aa, Human immunodeficiency virus
EELLKTVRLI

SEQ ID NO: 428 - protein, 10 aa, Human immunodeficiency virus
SELYKYKVVE

SEQ ID NO: 429 - protein, 10 aa, Human immunodeficiency virus
SELYKYKVVK

SEQ ID NO: 430 - protein, 10 aa, Human immunodeficiency virus
TEVHNVWATH

SEQ ID NO: 431 - protein, 10 aa, Human immunodeficiency virus
GEFFYCNTSG

SEQ ID NO: 432 - protein, 10 aa, Human immunodeficiency virus
IEAQQHLLQL

SEQ ID NO: 433 - protein, 10 aa, Human immunodeficiency virus
REKRAVGIGA

SEQ ID NO: 434 - protein, 10 aa, Human immunodeficiency virus
VEQMHEDIIS

SEQ ID NO: 435 - protein, 10 aa, Human immunodeficiency virus
RELERFAVNP

SEQ ID NO: 436 - protein, 10 aa, Human immunodeficiency virus
KEPFRDYVDR

SEQ ID NO: 437 - protein, 10 aa, Human immunodeficiency virus
AEQASQEVKN

SEQ ID NO: 438 - protein, 10 aa, Human immunodeficiency virus
AEQATQEVKN

SEQ ID NO: 439 - protein, 10 aa, Human immunodeficiency virus
GEAMHGQVDC

SEQ ID NO: 440 - protein, 10 aa, Human immunodeficiency virus
REILKEPVHG

SEQ ID NO: 441 - protein, 10 aa, Human immunodeficiency virus
NEQVDKLVSA

SEQ ID NO: 442 - protein, 10 aa, Human immunodeficiency virus
AEHLKTAVQM

SEQ ID NO: 443 - protein, 10 aa, Human immunodeficiency virus
EEKIKALVEI

SEQ ID NO: 444 - protein, 10 aa, Human immunodeficiency virus
PEKDSWTVND

SEQ ID NO: 445 - protein, 10 aa, Human immunodeficiency virus
IEAEVIPAET

SEQ ID NO: 446 - protein, 10 aa, Human immunodeficiency virus
RETKLGKAGY

SEQ ID NO: 447 - protein, 10 aa, Human immunodeficiency virus
DEELLKTVRL

SEQ ID NO: 448 - protein, 10 aa, Human immunodeficiency virus
MEPVDPRLEP

SEQ ID NO: 449 - protein, 10 aa, Human immunodeficiency virus
SESAIRNAIL

SEQ ID NO: 450 - protein, 10 aa, Human immunodeficiency virus
MENRWQVMIV

SEQ ID NO: 451 - protein, 10 aa, Human immunodeficiency virus
EELKSEAVRH

SEQ ID NO: 452 - protein, 10 aa, Human immunodeficiency virus
QEELSALVEM

SEQ ID NO: 453 - protein, 10 aa, Human immunodeficiency virus
SELYKYKVVE

SEQ ID NO: 454 - protein, 10 aa, Human immunodeficiency virus
SELYKYKVVK

SEQ ID NO: 455 - protein, 10 aa, Human immunodeficiency virus
QEVGKAMYAP

SEQ ID NO: 456 - protein, 10 aa, Human immunodeficiency virus
LELDKWASLW

SEQ ID NO: 457 - protein, 10 aa, Human immunodeficiency virus
FEPIPIHYCA

SEQ ID NO: 458 - protein, 10 aa, Human immunodeficiency virus
KEATTTLFCA

SEQ ID NO: 459 - protein, 10 aa, Human immunodeficiency virus
TEVHNVWATH

SEQ ID NO: 460 - protein, 10 aa, Human immunodeficiency virus
AEWDRLHPVH

SEQ ID NO: 461 - protein, 10 aa, Human immunodeficiency virus
EEKAFSPEVI

SEQ ID NO: 462 - protein, 10 aa, Human immunodeficiency virus
TETLLVQNAN

SEQ ID NO: 463 - protein, 10 aa, Human immunodeficiency virus
KETINEEAAE

SEQ ID NO: 464 - protein, 10 aa, Human immunodeficiency virus
TELQAIHLAL

SEQ ID NO: 465 - protein, 10 aa, Human immunodeficiency virus
AETFYVDGAA

SEQ ID NO: 466 - protein, 10 aa, Human immunodeficiency virus
TEEKIKALVE

SEQ ID NO: 467 - protein, 10 aa, Human immunodeficiency virus
KEKVYLAWVP

SEQ ID NO: 468 - protein, 10 aa, Human immunodeficiency virus
WEFVNTPPLV

SEQ ID NO: 469 - protein, 10 aa, Human immunodeficiency virus
TEEAELELAE

SEQ ID NO: 470 - protein, 10 aa, Human immunodeficiency virus
TEMEKEGKIS

SEQ ID NO: 471 - protein, 10 aa, Human immunodeficiency virus
LELAENREIL

SEQ ID NO: 472 - protein, 10 aa, Human immunodeficiency virus
EELLKTVRLI

SEQ ID NO: 473 - protein, 10 aa, Human immunodeficiency virus
RDWHLGQGVS

SEQ ID NO: 474 - protein, 10 aa, Human immunodeficiency virus
MENRWQVMIV

SEQ ID NO: 475 - protein, 10 aa, Human immunodeficiency virus
YETYGDTWAG

SEQ ID NO: 476 - protein, 10 aa, Human immunodeficiency virus
EELSALVEMG

SEQ ID NO: 477 - protein, 10 aa, Human immunodeficiency virus
LELDKWASLW

SEQ ID NO: 478 - protein, 10 aa, Human immunodeficiency virus
LEITTHSFNC

SEQ ID NO: 479 - protein, 10 aa, Human immunodeficiency virus
DNWRSELYKY

SEQ ID NO: 480 - protein, 10 aa, Human immunodeficiency virus
KEATTTLFCA

SEQ ID NO: 481 - protein, 10 aa, Human immunodeficiency virus
GDLEITTHSF

SEQ ID NO: 482 - protein, 10 aa, Human immunodeficiency virus
IEAQQHLLQL

SEQ ID NO: 483 - protein, 10 aa, Human immunodeficiency virus
QARVLAVERY

SEQ ID NO: 484 - protein, 10 aa, Human immunodeficiency virus
GEIYKRWIIL

SEQ ID NO: 485 - protein, 10 aa, Human immunodeficiency virus
LGLNKIVRMY

SEQ ID NO: 486 - protein, 10 aa, Human immunodeficiency virus
EEQNKSKKKA

SEQ ID NO: 487 - protein, 10 aa, Human immunodeficiency virus
QEVKNWMTET

SEQ ID NO: 488 - protein, 10 aa, Human immunodeficiency virus
KEPPFLWMGY

SEQ ID NO: 489 - protein, 10 aa, Human immunodeficiency virus
NETPGIRYQY

SEQ ID NO: 490 - protein, 10 aa, Human immunodeficiency virus
AETGQETAYF

SEQ ID NO: 491 - protein, 10 aa, Human immunodeficiency virus
RELNKRTQDF

SEQ ID NO: 492 - protein, 10 aa, Human immunodeficiency virus
RETKLGKAGY

SEQ ID NO: 493 - protein, 10 aa, Human immunodeficiency virus
LEIGQHRTKI

SEQ ID NO: 494 - protein, 10 aa, Human immunodeficiency virus
EPIVGAETFY

SEQ ID NO: 495 - protein, 10 aa, Human immunodeficiency virus
TEMEKEGKIS

SEQ ID NO: 496 - protein, 10 aa, Human immunodeficiency virus
EELLKTVRLI

SEQ ID NO: 497 - protein, 10 aa, Human immunodeficiency virus
QPKTACTNCY

SEQ ID NO: 498 - protein, 10 aa, Human immunodeficiency virus
GDARLVITTY

SEQ ID NO: 499 - protein, 10 aa, Human immunodeficiency virus
GDAKLVITTY

SEQ ID NO: 500 - protein, 10 aa, Human immunodeficiency virus
EDQGPQREPY

SEQ ID NO: 501 - protein, 10 aa, Human immunodeficiency virus
IAIVVWTIVF

SEQ ID NO: 502 - protein, 10 aa, Human immunodeficiency virus
LPCRIKQIIN

SEQ ID NO: 503 - protein, 10 aa, Human immunodeficiency virus
CPKVSFEPIP

SEQ ID NO: 504 - protein, 10 aa, Human immunodeficiency virus
VAEGTDRVIE

SEQ ID NO: 505 - protein, 10 aa, Human immunodeficiency virus
APTKAKRRVV

SEQ ID NO: 506 - protein, 10 aa, Human immunodeficiency virus
APTRAKRRVV

SEQ ID NO: 507 - protein, 10 aa, Human immunodeficiency virus
GPCKNVSTVQ

SEQ ID NO: 508 - protein, 10 aa, Human immunodeficiency virus
GPCTNVSTVQ

SEQ ID NO: 509 - protein, 10 aa, Human immunodeficiency virus
NPPIPVGEIY

SEQ ID NO: 510 - protein, 10 aa, Human immunodeficiency virus
NPPIPVGDIY

SEQ ID NO: 511 - protein, 10 aa, Human immunodeficiency virus
NANPDCKTIL

SEQ ID NO: 512 - protein, 10 aa, Human immunodeficiency virus
SPRTLNAWVK

SEQ ID NO: 513 - protein, 10 aa, Human immunodeficiency virus
FPISPIETVP

SEQ ID NO: 514 - protein, 10 aa, Human immunodeficiency virus
LPEKDSWTVN

SEQ ID NO: 515 - protein, 10 aa, Human immunodeficiency virus
WASQIYAGIK

SEQ ID NO: 516 - protein, 10 aa, Human immunodeficiency virus
TAVQMAVFIH

SEQ ID NO: 517 - protein, 10 aa, Human immunodeficiency virus
QGWKGSPAIF

SEQ ID NO: 518 - protein, 10 aa, Human immunodeficiency virus
SGYIEAEVIP

SEQ ID NO: 519 - protein, 10 aa, Human immunodeficiency virus
QPDKSESELV

SEQ ID NO: 520 - protein, 10 aa, Human immunodeficiency virus
GPKVKQWPLT

SEQ ID NO: 521 - protein, 10 aa, Human immunodeficiency virus
LPPLERLTLD

SEQ ID NO: 522 - protein, 10 aa, Human immunodeficiency virus
GPKESKKKVE

SEQ ID NO: 523 - protein, 10 aa, Human immunodeficiency virus
DPDLADQLIH

SEQ ID NO: 524 - protein, 10 aa, Human immunodeficiency virus
DPGLADQLIH

SEQ ID NO: 525 - protein, 10 aa, Human immunodeficiency virus
EAVRHFPRIW

SEQ ID NO: 526 - protein, 10 aa, Human immunodeficiency virus
QPLVILAIVA

SEQ ID NO: 527 - protein, 10 aa, Human immunodeficiency virus
APTKAKRRVV

SEQ ID NO: 528 - protein, 10 aa, Human immunodeficiency virus
APTRAKRRVV

SEQ ID NO: 529 - protein, 10 aa, Human immunodeficiency virus
LPCRIKQIIN

SEQ ID NO: 530 - protein, 10 aa, Human immunodeficiency virus
KPVVSTQLLL

SEQ ID NO: 531 - protein, 10 aa, Human immunodeficiency virus
RPVVSTQLLL

SEQ ID NO: 532 - protein, 10 aa, Human immunodeficiency virus
GPCKNVSTVQ

SEQ ID NO: 533 - protein, 10 aa, Human immunodeficiency virus
GPCTNVSTVQ

SEQ ID NO: 534 - protein, 10 aa, Human immunodeficiency virus
NPPIPVGEIY

SEQ ID NO: 535 - protein, 10 aa, Human immunodeficiency virus
NPPIPVGDIY

SEQ ID NO: 536 - protein, 10 aa, Human immunodeficiency virus
NANPDCKTIL

SEQ ID NO: 537 - protein, 10 aa, Human immunodeficiency virus
NANPDCKSIL

SEQ ID NO: 538 - protein, 10 aa, Human immunodeficiency virus
FPISPIETVP

SEQ ID NO: 539 - protein, 10 aa, Human immunodeficiency virus
TAVQMAVFIH

SEQ ID NO: 540 - protein, 10 aa, Human immunodeficiency virus
QGWKGSPAIF

SEQ ID NO: 541 - protein, 10 aa, Human immunodeficiency virus
SGYIEAEVIP

SEQ ID NO: 542 - protein, 10 aa, Human immunodeficiency virus
KPGMDGPKVK

SEQ ID NO: 543 - protein, 10 aa, Human immunodeficiency virus
GGIGGFIKVR

SEQ ID NO: 544 - protein, 10 aa, Human immunodeficiency virus
WASQIYAGIK

SEQ ID NO: 545 - protein, 10 aa, Human immunodeficiency virus
KGIGGNEQVD

SEQ ID NO: 546 - protein, 10 aa, Human immunodeficiency virus
LPPLERLTLD

SEQ ID NO: 547 - protein, 10 aa, Human immunodeficiency virus
GPKESKKKVE

SEQ ID NO: 548 - protein, 10 aa, Human immunodeficiency virus
IPLGDARLVI

SEQ ID NO: 549 - protein, 10 aa, Human immunodeficiency virus
IPLGDAKLVI

SEQ ID NO: 550 - protein, 10 aa, Human immunodeficiency virus
EAVRHFPRIW

SEQ ID NO: 551 - protein, 10 aa, Human immunodeficiency virus
QPLVILAIVA

SEQ ID NO: 552 - protein, 10 aa, Human immunodeficiency virus
VTVYYGVPVW

SEQ ID NO: 553 - protein, 10 aa, Human immunodeficiency virus
ITQACPKVSF

SEQ ID NO: 554 - protein, 10 aa, Human immunodeficiency virus
HSFNCGGEFF

SEQ ID NO: 555 - protein, 10 aa, Human immunodeficiency virus
HSFNCRGEFF

SEQ ID NO: 556 - protein, 10 aa, Human immunodeficiency virus
VSFEPIPIHY

SEQ ID NO: 557 - protein, 10 aa, Human immunodeficiency virus
ITLPCRIKQI

SEQ ID NO: 558 - protein, 10 aa, Human immunodeficiency virus
CSGKLICTTA

SEQ ID NO: 559 - protein, 10 aa, Human immunodeficiency virus
TSTLQEQIGW

SEQ ID NO: 560 - protein, 10 aa, Human immunodeficiency virus
ETINEEAAEW

SEQ ID NO: 561 - protein, 10 aa, Human immunodeficiency virus
DTINEEAAEW

SEQ ID NO: 562 - protein, 10 aa, Human immunodeficiency virus
PSHKGRPGNF

SEQ ID NO: 563 - protein, 10 aa, Human immunodeficiency virus
VSAGIRKVLF

SEQ ID NO: 564 - protein, 10 aa, Human immunodeficiency virus
WTYQIYQEPF

SEQ ID NO: 565 - protein, 10 aa, Human immunodeficiency virus
STKWRKLVDF

SEQ ID NO: 566 - protein, 10 aa, Human immunodeficiency virus
SSMTKILEPF

SEQ ID NO: 567 - protein, 10 aa, Human immunodeficiency virus
QATWIPEWEF

SEQ ID NO: 568 - protein, 10 aa, Human immunodeficiency virus
NTPPLVKLWY

SEQ ID NO: 569 - protein, 10 aa, Human immunodeficiency virus
MGYELHPDKW

SEQ ID NO: 570 - protein, 10 aa, Human immunodeficiency virus
ISKIGPENPY

SEQ ID NO: 571 - protein, 10 aa, Human immunodeficiency virus
QARRNRRRRW

SEQ ID NO: 572 - protein, 10 aa, Human immunodeficiency virus
FTKKGLGISY

SEQ ID NO: 573 - protein, 10 aa, Human immunodeficiency virus
DARLVITTYW

SEQ ID NO: 574 - protein, 10 aa, Human immunodeficiency virus
DAKLVITTYW

SEQ ID NO: 575 - protein, 10 aa, Human immunodeficiency virus
EAVRHFPRIW

SEQ ID NO: 576 - protein, 10 aa, Human immunodeficiency virus
VAAIIAIVVW

SEQ ID NO: 577 - protein, 10 aa, Human immunodeficiency virus
NAKTIIVQLN

SEQ ID NO: 578 - protein, 10 aa, Human immunodeficiency virus
TLPCRIKQII

SEQ ID NO: 579 - protein, 10 aa, Human immunodeficiency virus
CAPAGFAILK

SEQ ID NO: 580 - protein, 10 aa, Human immunodeficiency virus
QLQARVLAVE

SEQ ID NO: 581 - protein, 10 aa, Human immunodeficiency virus
LTVWGIKQLQ

SEQ ID NO: 582 - protein, 10 aa, Human immunodeficiency virus
EAQQHLLQLT

SEQ ID NO: 583 - protein, 10 aa, Human immunodeficiency virus
QLLSGIVQQQ

SEQ ID NO: 584 - protein, 10 aa, Human immunodeficiency virus
IWPSHKGRPG

SEQ ID NO: 585 - protein, 10 aa, Human immunodeficiency virus
RAPRKKGCWK

SEQ ID NO: 586 - protein, 10 aa, Human immunodeficiency virus
TLQEQIGWMT

SEQ ID NO: 587 - protein, 10 aa, Human immunodeficiency virus
FLQSRPEPTA

SEQ ID NO: 588 - protein, 10 aa, Human immunodeficiency virus
KALTEVIPLT

SEQ ID NO: 589 - protein, 10 aa, Human immunodeficiency virus
NLKTGKYARM

SEQ ID NO: 590 - protein, 10 aa, Human immunodeficiency virus
GAANRETKLG

SEQ ID NO: 591 - protein, 10 aa, Human immunodeficiency virus
WVPAHKGIGG

SEQ ID NO: 592 - protein, 10 aa, Human immunodeficiency virus
LEPFRKQNPD

SEQ ID NO: 593 - protein, 10 aa, Human immunodeficiency virus
KEPVHGVYYD

SEQ ID NO: 594 - protein, 10 aa, Human immunodeficiency virus
ELAENREILK

SEQ ID NO: 595 - protein, 10 aa, Human immunodeficiency virus
GGNEQVDKLV

SEQ ID NO: 596 - protein, 10 aa, Human immunodeficiency virus
ILVESPTVLE

SEQ ID NO: 597 - protein, 10 aa, Human immunodeficiency virus
DSQTHQASLS

SEQ ID NO: 598 - protein, 10 aa, Human immunodeficiency virus
PLPSVKKLTE

SEQ ID NO: 599 - protein, 10 aa, Human immunodeficiency virus
HTGERDWHLG

SEQ ID NO: 600 - protein, 10 aa, Human immunodeficiency virus
QAPEDQGPQR

SEQ ID NO: 601 - protein, 10 aa, Human immunodeficiency virus
ILRQRKIDRL

SEQ ID NO: 602 - protein, 10 aa, Human immunodeficiency virus
KYWWNLLQYW

SEQ ID NO: 603 - protein, 10 aa, Human immunodeficiency virus
LRSLCLFSYH

SEQ ID NO: 604 - protein, 10 aa, Human immunodeficiency virus
ARVLAVERYL

SEQ ID NO: 605 - protein, 10 aa, Human immunodeficiency virus
SYHRLRDLLL

SEQ ID NO: 606 - protein, 10 aa, Human immunodeficiency virus
FNCGGEFFYC

SEQ ID NO: 607 - protein, 10 aa, Human immunodeficiency virus
IRPVVSTQLL

SEQ ID NO: 608 - protein, 10 aa, Human immunodeficiency virus
IRQGLERALL

SEQ ID NO: 609 - protein, 10 aa, Human immunodeficiency virus
LRPGGKKKYR

SEQ ID NO: 610 - protein, 10 aa, Human immunodeficiency virus
LYNTVATLYC

SEQ ID NO: 611 - protein, 10 aa, Human immunodeficiency virus
FSPEVIPMFS

SEQ ID NO: 612 - protein, 10 aa, Human immunodeficiency virus
IRQGPKEPFR

SEQ ID NO: 613 - protein, 10 aa, Human immunodeficiency virus
TPPLVKLWYQ

SEQ ID NO: 614 - protein, 10 aa, Human immunodeficiency virus
KRKGGIGGYS

SEQ ID NO: 615 - protein, 10 aa, Human immunodeficiency virus
IYQYMDDLYV

SEQ ID NO: 616 - protein, 10 aa, Human immunodeficiency virus
EPPFLWMGYE

SEQ ID NO: 617 - protein, 10 aa, Human immunodeficiency virus
TVLDVGDAYF

SEQ ID NO: 618 - protein, 10 aa, Human immunodeficiency virus
QYALGIIQAQ

SEQ ID NO: 619 - protein, 10 aa, Human immunodeficiency virus
LKEPVHGVYY

SEQ ID NO: 620 - protein, 10 aa, Human immunodeficiency virus
KQGQGQWTYQ

SEQ ID NO: 621 - protein, 10 aa, Human immunodeficiency virus
LQLPPLERLT

SEQ ID NO: 622 - protein, 10 aa, Human immunodeficiency virus
LNKGLGISYG

SEQ ID NO: 623 - protein, 10 aa, Human immunodeficiency virus
QYLALAALIK

SEQ ID NO: 624 - protein, 10 aa, Human immunodeficiency virus
QYLALAALIT

SEQ ID NO: 625 - protein, 10 aa, Human immunodeficiency virus
LHGLGQHIYE

SEQ ID NO: 626 - protein, 10 aa, Human immunodeficiency virus
VWTIVFIEYR

SEQ ID NO: 627 - protein, 10 aa, Human immunodeficiency virus
RPNNNTRKSI

SEQ ID NO: 628 - protein, 10 aa, Human immunodeficiency virus
NPYNTPIFAL

SEQ ID NO: 629 - protein, 10 aa, Human immunodeficiency virus
RAIEAQQHLL

SEQ ID NO: 630 - protein, 10 aa, Human immunodeficiency virus
TCKSNITGLL

SEQ ID NO: 631 - protein, 9 aa, Human immunodeficiency virus
KPVVSTQLL

SEQ ID NO: 632 - protein, 9 aa, Human immunodeficiency virus
KPCVKLTPL

SEQ ID NO: 633 - protein, 10 aa, Human immunodeficiency virus
KPCVKLTPLC

SEQ ID NO: 634 - protein, 9 aa, Human immunodeficiency virus
GPKVKQWPL

SEQ ID NO: 635 - protein, 10 aa, Human immunodeficiency virus
GPKVKQWPLT

SEQ ID NO: 636 - protein, 9 aa, Human immunodeficiency virus
YPGIKVRQL

SEQ ID NO: 637 - protein, 10 aa, Human immunodeficiency virus
YPGIKVRQLC

SEQ ID NO: 638 - protein, 10 aa, Human immunodeficiency virus
TVLDVGDAYF

SEQ ID NO: 639 - protein, 9 aa, Human immunodeficiency virus
EPPFLWMGY

SEQ ID NO: 640 - protein, 10 aa, Human immunodeficiency virus
EPPFLWMGYE

SEQ ID NO: 641 - protein, 9 aa, Human immunodeficiency virus
VPVKLKPGM

SEQ ID NO: 642 - protein, 10 aa, Human immunodeficiency virus
VPVKLKPGMD

SEQ ID NO: 643 - protein, 9 aa, Human immunodeficiency virus
CPKVTFDPI

SEQ ID NO: 644 - protein, 10 aa, Human immunodeficiency virus
CPKVTFDPIP

SEQ ID NO: 645 - protein, 9 aa, Human immunodeficiency virus
KPVVSTQLL

SEQ ID NO: 646 - protein, 9 aa, Human immunodeficiency virus
KPCVKLTPL

SEQ ID NO: 647 - protein, 10 aa, Human immunodeficiency virus
KPCVKLTPLC

SEQ ID NO: 648 - protein, 9 aa, Human immunodeficiency virus
GPKVKQWPL

SEQ ID NO: 649 - protein, 10 aa, Human immunodeficiency virus
GPKVKQWPLT

SEQ ID NO: 650 - protein, 9 aa, Human immunodeficiency virus
YPGIKVRQL

SEQ ID NO: 651 - protein, 10 aa, Human immunodeficiency virus
YPGIKVRQLC

SEQ ID NO: 652 - protein, 9 aa, Human immunodeficiency virus
ILKEPVHGV

SEQ ID NO: 653 - protein, 10 aa, Human immunodeficiency virus
ILKEPVHGVY

SEQ ID NO: 654 - protein, 10 aa, Human immunodeficiency virus
QLPEKDSWTV

SEQ ID NO: 655 - protein, 9 aa, Human immunodeficiency virus
NLWTVYYGV

SEQ ID NO: 656 - protein, 9 aa, Human immunodeficiency virus
QMHEDVISL

SEQ ID NO: 657 - protein, 10 aa, Human immunodeficiency virus
QMHEDVISLW

SEQ ID NO: 658 - protein, 10 aa, Human immunodeficiency virus
KIEELREHLL

SEQ ID NO: 659 - protein, 10 aa, Human immunodeficiency virus
DMVNQMHEDV

SEQ ID NO: 660 - protein, 10 aa, Human immunodeficiency virus
GLKKKKSVTV

SEQ ID NO: 661 - protein, 9 aa, Human immunodeficiency virus
ELHPDKWTV

SEQ ID NO: 662 - protein, 10 aa, Human immunodeficiency virus
IYQEPFKNLK

SEQ ID NO: 663 - protein, 10 aa, Human immunodeficiency virus
VTFDPIPIHY

SEQ ID NO: 664 - protein, 9 aa, Human immunodeficiency virus
TVQCTHGIK

SEQ ID NO: 665 - protein, 10 aa, Human immunodeficiency virus
TVQCTHGIKP

SEQ ID NO: 666 - protein, 10 aa, Human immunodeficiency virus
NTPIFALKKK

SEQ ID NO: 667 - protein, 9 aa, Human immunodeficiency virus
LVDFRELNK

SEQ ID NO: 668 - protein, 10 aa, Human immunodeficiency virus
LVDFRELNKR

SEQ ID NO: 669 - protein, 9 aa, Human immunodeficiency virus
PGMDGPKVK

SEQ ID NO: 670 - protein, 10 aa, Human immunodeficiency virus
PGMDGPKVKQ

SEQ ID NO: 671 - protein, 10 aa, Human immunodeficiency virus
GIPHPAGLKK

SEQ ID NO: 672 - protein, 10 aa, Human immunodeficiency virus
FTTPDKKHQK

(19) United States
(12) Patent Application Publication
(10) Pub. No.: US 2003/0180314 A1
(43) Pub. Date: Sep. 25, 2003

(54) Immunogenic, cross-clade, HIV peptides

(76) Inventor: Anne DeGroot, Providence, RI (US)

Correspondence Address: LUANN CSERR, LAW OFFICE OF LUANN CSERR, SUITE 100, 166 WHEELER AVENUE, CRANSTON, RI, 02905-2710, US

(21) Appl. No.: 10/200,708

(22) Filed: Jul. 22, 2002

Related U.S. Application Data
(63) Continuation-in-part of application No. 09/351,036, filed on Jul. 9, 1999, now abandoned.
(60) Provisional application No. 60/092,346, filed on Jul. 10, 1998.
(60) Provisional application No. 60/115,145, filed on Jan. 8, 1999.
(60) Provisional application No. 60/130,677, filed on Apr. 23, 1999.

(51) Int. Cl. A61K 39/29; A61K 39/21; C07K 14/16

(52) U.S. Cl. 424/188.1; 530/350

(57) ABSTRACT

The invention provides Cross-clade candidates that have “evolved” due to gene shuffling in vitro for inclusion of “cross-clade” characteristics. The invention also provides a method for identifying Cross-clade candidates that could be presented in the context of more than one HLA, due to the creation of promiscuous epitopes by gene shuffling.

### CLAIM OF PRIORITY

This application claims priority under 35 U.S.C. §119(e) to U.S. provisional patent applications No. 60/092,346, filed Jul. 10, 1998; No. 60/115,145, filed Jan. 8, 1999; and No. 60/130,677, filed Apr. 23, 1999. This application is a continuation-in-part of U.S. Ser. No. 09/351,036 filed Jul. 9, 1999 and claims priority therefrom.

### STATEMENT REGARDING FEDERALLY SPONSORED RESEARCH

This invention was made with United States Government support from the National Institutes of Health. The Government may have certain rights in the invention.

### TECHNICAL FIELD OF THE INVENTION

This invention concerns the treatment and prevention of viral infections in humans. More specifically, this invention relates to the treatment and prevention of human immunodeficiency virus 1 (HIV-1) infections.

### BACKGROUND OF THE INVENTION

The need for an effective treatment (therapeutic or prophylactic) against human immunodeficiency virus type 1 (HIV-1) remains urgent. The great diversity in the genetic composition of the HIV-1 virus combined with the absolute specificity of the human cytotoxic T cell (CTL) response is an important factor responsible for the lack of development of an effective vaccine. Numerous strains (“clades”) of HIV-1 have been identified. These clades exhibit significant differences from each other in nucleotide sequence, which results in significant differences in amino acid sequences among the clades. The vast majority of the 16,000 new HIV-1 infections that occur every day are acquired by individuals who live in developing countries, where the isolates of HIV that are transmitted are significantly different from the isolates selected for most of the HIV-1 vaccines currently under development. HIV-1 subtypes, or clades, A, C, and D predominate in most of sub-Saharan Africa, lade E (AE) is the most prevalent in Thailand, and new A/G chimeras are emerging in West Africa. See, DeGroot et al., Mapping Cross-clade HIV-1 Epitopes Using Bioinformatics, manuscript in preparation. Recent research indicates that regional clusters within subtypes exist; for example, isolates within lade C that circulate in South Africa differ significantly from isolates within lade C that circulate in India.

Despite the predominance of non-clade B isolates in the global epidemic, most researchers developing HIV vaccines have focused on defining the immune responses against one particular vaccine candidate. Most test HIV vaccines currently in Phase I through Phase III clinical trials target the group of lade B strains of HIV. In other words, such vaccines are designed to elicit an immune response to HIV viruses belonging to the clade B subgroup. Some of these vaccine candidates are derived from lab strains of HIV, others are derived from lade B patient isolates. “Challenge” strains of HIV, those strains known to exist in the United States to which immunized individuals may be exposed, may be 10 to 15% different from the strains used to develop these vaccines. Challenge strains in other regions of the world, and new strains arriving in the United States from other regions of the world, may exhibit even more sequence divergence from the strains used to develop these vaccines. There is roughly 15-20% divergence between the nucleic acid sequences of different clades and approximately 7-12% variation within a lade. Due to such variations, the body's immune response raised against one vaccine strain may not protect against other strains of HIV. Researchers have yet to achieve the development of an HIV vaccine that will stimulate an effective immune response to more than one HIV clade.

The characteristic specificity of the interaction between viral protein sequences and the molecules of the human immune system (the human leukocyte antigens or “HLA”) is responsible for this problem. The HLA molecules of the major histocompatibility complex (MHC) present peptides derived from viral proteins to T lymphocyte cells (“T cells”), eliciting the engagement of the T cells in fighting and eliminating the virus. Certain T cells are cytotoxic T lymphocytes (CTL), which have the ability to kill cells that have foreign molecules on their surfaces. The HLA molecules, which are typically proteins present on the surface of Antigen Presenting Cells (“APCs”) such as B lymphocytes, dendricytes and macrophages, non-covalently bind to these virus-derived peptides. This binding is necessary for the T cell to be able to recognize the peptide as viral, which it does through receptor proteins (T cell receptors) on it surface. Small changes in the amino acid sequence of the viral peptide may prevent the binding of it to the HLA molecule and deleteriously affect recognition of the virus strain by the T cells. Sequence modifications at the amino acid level may affect recognition of the epitope by affecting intracellular processing, by interfering with the binding of the peptide to HLA molecules (HLA) and presentation of the peptide-HLA complex at the antigen presenting-cell surface, and/or by interfering with the binding of the epitope to the T cell receptor (TCR). See Germain & Margulies, 11 Ann. Rev. Immunol. 403 (1993); Falk et al., 351 Nature 290 (1991). See for general background, Stites et al., Basic & Clinical Immunology, 8^{th} Ed, Appleton & Lange, Stamford, 1994. Thus, changes in amino acid sequence associated with HIV-1 diversity may prevent cross-clade protection against HIV-1 challenge by T cell clones raised against dade B vaccine constructs. Viral escape from immune detection has been linked to amino acid substitution in HIV-1 T cell epitopes. Thus, immunization with vaccines containing epitopes derived exclusively from dade B may not protect against challenge by HIV-1 isolates that are divergent, at the epitope level, from the vaccine strain.

Cross-clade recognition of HIV epitopes has been studied in the art. For examples, see Wilson et al., 14(11) AIDS Res. Hum. Retroviruses 925-37 (1998); McAdam et al., 12(6) AIDS .571-9 (1998); Lynch et al., 178(4) J Infect Dis. 1040-6 (1998); Boyer et al., 95 Dev. Biol. Stand. 147-53 (1998); Cao et al., 71(11) J. Virol. 8615-23 (1997); and Durali et al., 72(5) Virol. 3547 53 (1998)). In general, these studies used vaccinia-expressed constructs containing the entire HIV genome to probe CTL lines from HIV-1 infected or HIV-1 vaccinated volunteers for CTL responses. For that reason, what appeared to be cross-clade recognition by CTL may have actually been recognition of CTL epitopes conserved within the large gene constructs cloned into the vaccinia virus and the vaccine strain or the autologous strain. In experiments in which responses to specific peptides and their altered sequences in other HIV strains have been tested, and in which the peptides have been mapped, studies have shown a lack of cross-strain recognition. See Dorrel et al., HIV Vaccine Development Opportunities And Challenges Meeting, Abstract 109 (Keystone, Colorado, January 1999). Studies of virus escape from CTL recognition carried out on HIV-1 infected individuals have also shown that viral variation at the amino acid level may abrogate effective CTL responses. See Koup, 180 J. Exp. Med. 779 (1994); Dai et al., 66 J. Virol. 3151 (1992); Johnson et al., 175 J. Exp. Med. 961 (1992).

In sum, no single HIV strain has been found yet that will stimulate effective HLA-restricted immune response against a wide range of HIV strains. HIV-1 vaccines that include highly conserved and immunogenic regions of the HIV-1 genome would likely be the most effective types of vaccine in the global context of the HIV epidemic. Preferred immunogenic regions to include in vaccine constructs would be cytotoxic T cell epitopes, since CTL response to HIV-1 epitopes contributes to protection both prior to infection and after exposure. Discovery of highly conserved sequences that are also immunogenic has been hampered by the lack of means to screen the large number of possible epitopes in the HIV-1 genome, as more than 55,000 HIV-1 protein sequences representing the eight clades of HIV-1 have been filed in public databases. Directly evaluating each overlapping peptide in this vast database of sequences would require the synthesis of millions of peptides and blood samples from thousands of volunteers. There remains a need in the art for a “world lade” HIV vaccine, a vaccine that will stimulate effective immune responses to more than one lade of HIV. And there remains a need for a more rapid approach to identifying highly conserved HIV-1 epitopes.

### SUMMARY OF THE INVENTION

In one aspect, the invention provides cross-clade candidate peptides not heretofore recognized or known in the art. By “cross-clade” we mean able to elicit an effective immune response to infection or challenge by HIV isolates belonging to more than one HIV clade (or subtype of HIV); i.e., at least two different isolates from different clades. These peptides were identified by screening a large database of HIV isolate protein sequences (the entire list of HIV-1 sequences available in the 1997 version of the Los Alamos National Laboratory HIV Sequence Database site [LINL}) for strings of amino acids (peptides) that were conserved in many of these isolates and usually in more than one clade. The conserved peptides were then evaluated for potential to bind to HLA molecules of the MHC, and those that were likely to bind to one or more HLA molecule were selected.

These peptide sequences are characterized by:

(i) comprising between eight and fifty amino acids;

(ii) having complete sequence identity with a partial HIV-1 amino acid sequence that is absolutely conserved across at least 2 strains of HIV; and possessing at least one of the biological properties selected from the group consisting of:

(iii) the ability to bind to a human HLA molecule based on possession of amino acid patterns that conform to a MHC binding matrix motif for a human HLA molecule of the MHC;

(iv) the ability to bind to a human HLA molecule in the T2 in vitro peptide binding assay, as demonstrated by exhibition of greater than 1.3-fold increase in MFI (mean fold increase) upon FACS (fluorescence-activated cell sorter) analysis; and

(v) the ability to activate T cells from HIV positive patients in at least one in vitro assay selected from the group consisting of the ELIspot T cell assay, the ELIspot T cell restimulation assay, T cell proliferation assays, intracellular cytokine staining assays, the Brefeldin incorporation assay and tetramer staining technique.

A human MHC binding matrix motif for a human MHC allele is a quantitative estimation of the relative ability of an amino acid in a given sequence to non-covalently bind to another amino acid. Such motifs are generally derived from lists of peptides known to bind to a given HLA molecule and are restricted by the corresponding MHC allele, as described later in the specification.

More specifically, the peptide sequences are characterized as having between eight and twenty-five amino acids, preferably between eight and eleven amino acids. The peptides can be any size between the specified minimums and maximums independently; for example, one cross-clade candidate peptide may comprise eight amino acids and another may comprise eleven or fifteen amino acids.

Even more specifically, the HIV cross-clade candidate peptides exhibit complete sequence identity to a partial HIV-1 amino acid sequence from any of the proteins of HIV-1, for example, from the env, pol, nef, vif, vpu, vpx, vpr or tat proteins of HIV-1, and the HLA allele to which they bind is an HLA-A2 or an HLA-B7 allele.

Most specifically, the HIV cross-clade candidate peptides comprise sequences corresponding to the HIV peptides shown in any of FIG. 2 (SEQ ID NO:1-27), TABLES 6-31 (SEQ ID NO: 28-626); and FIGS. 6-9 and TABLE 1-4 (SEQ ID NO:627-672). Such sequences correspond to HIV protein sequences obtained from the Los Alamos HIV Sequence Database.

In another aspect, the invention provides polynucleotide sequences encoding the cross-clade candidate peptides. The polynucleotide can be a recombinant construct such as a vector or plasmid that contains the encoding polynucleotide sequence, alone or as a fusion protein, under the operative control of polynucleotides encoding regulatory elements such as promoters, termination signals, and the like. Additionally provided by this invention is a recombinant polynucleotide vector comprising vector nucleotides and polynucleotide sequences encoding cross-clade candidate peptides in operative association with a regulatory sequence capable of directing the replication and expression of the polynucleotide sequence encoding the cross-clade candidate peptide in a selected host cell. Host cells transformed with such vectors for use in expressing recombinant cross-clade peptides are also provided by this invention. Also provided is a process for producing recombinant cross-clade peptides. In this process, a host cell line, transformed with a vector as described above containing a polynucleotide sequence encoding the cross-clade peptide in operative association with a suitable regulatory sequence capable of directing replication and controlling expression of the sequence, is cultured under appropriate conditions permitting expression of the recombinant polynucleotide. The expression peptide is then harvested from the host cell or culture medium using suitable conventional means. This process may employ various known cells as hosts cell lines for expression of the peptide.

The cross-clade peptide sequences of this invention may be used to prepare therapeutic and/or immunogenic compositions for preventing and treating HIV infection. Such pharmaceutical compositions comprise an immunogenically-inducing effective amount of at least one cross-clade candidate peptide in admixture with an immunologically acceptable excipient. Preferably, such pharmaceutical compositions comprise an immunogenically-inducing effective amount of more than one cross-clade candidate peptide in admixture with an immunologically acceptable excipient. We anticipate that a cocktail of cross-clade peptides, exhibiting different or overlapping clade identities, may be advantageously employed. The cross-clade candidate peptide(s) may be combined with or linked to a suitable carrier such as a carrier protein or may be expressed from a polynucleotide, in a “naked DNA” vaccine. In the latter case, the composition will comprise an immunogenically-inducing effective amount of the polynucleotide(s) in admixture with an immunologically acceptable excipient.

Additionally provided is a method of preventing or treating HIV infection. In practicing the method of treatment, an immunologically-inducing effective amount of peptide sequence(s) or polynucleotide sequence(s) is administered to a human patient in need of therapeutic or prophylactic treatment.

An immunologically-inducing effective amount is contemplated to be in the range of between about 50 μg to about 1 mg of the cross-clade candidate peptide per ml of a sterile solution. A more preferred dosage can be about 200 μg of cross-clade candidate peptide per dose administered.

In yet another aspect, the invention provides a method for identifying cross-clade immunogenic HIV peptide candidates. Such candidates could be presented in the context of more than one HLA due to the creation of promiscuous epitopes by gene shuffling. In the method, cross-clade HIV peptides are first identified. A “cross-clade” HIV peptide is an HIV peptide conserved across at least two HIV strains. Next, the identified HIV peptides are analyzed for being putative ligands for HLA molecules. Ligands that are highly likely to bind to one or more HLA molecules are identified and tested for binding in vitro and then for immunogenicity in vitro. Ligands demonstrating immunogenicity are cross-clade immunogenic HIV peptide candidates.

In another aspect, the invention provides antibodies raised against the cross-clade candidate peptides of the invention. The antibodies may include polyclonal antibodies, produced by immunizing a mammal with the peptide immunogen, monoclonal antibodies, chimeric antibodies, humanized antibodies and fully human antibodies. The antibodies raised are isolated and purified from the plasma, serum or culture medium conventional techniques. Such antibodies can themselves be employed as pharmaceutical compositions of this invention. Other antibodies can be developed by screening hybridomas or combinatorial libraries, or antibody phage displays (see Huse et al., 246 Science 1275-1281 (1988) using the antibodies produced according to this invention and the amino acid sequences of the primary or optional immunogens.

Other aspects and advantages of this invention are described in the following detailed description.

### BRIEF DESCRIPTION OF THE DRAWINGS

FIG. 1 is a histogram illustration showing the distribution of the number of HIV-1 isolates in which 8-mer to 11-mer peptides predicted to bind (A) and (b) HLA-B27 are exactly conserved.

FIG. 2 is a table illustration containing the results for the 8-mer to 11-mer candidate peptides synthesized and tested in Example 1. The second and third columns contain the estimated binding probability for the delineated 8-11-mer peptides for HLA A2 and B27 ligands having EpiMatrix scores at least as high as these peptides. The fourth and fifth columns indicate the highest fold-change in MFI for concentrations over 1.3. The sixth column indicates the protein of origin. The seventh column indicates the number of HIV-1 isolate sequences containing the amino acid sequence set forth in the first column. The eighth column indicates the approximate position of the sequence relative to the LAI reference strain. The ninth through fourteenth columns indicate the HIV lade to which the sequence belongs. The fifteenth column indicates the sequence identification number corresponding to the vaccine candidate peptide sequences set forth in column one.

FIG. 3 is a flow diagram illustration showing a project outline for identifying regional cross-clade candidate peptides.

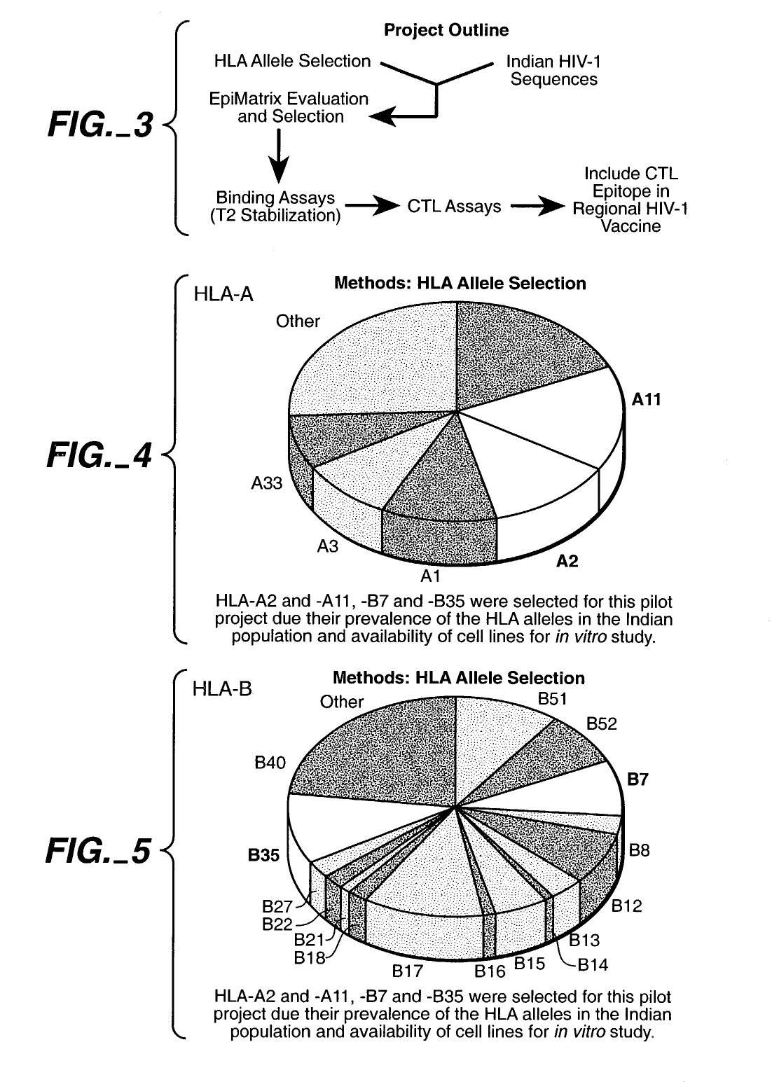

FIGS. 4-5 are pie chart illustrations showing the relative percentages of certain HLA-A (FIG. 4) and HLA-B (FIG. 5) alleles in the Indian population and the alleles selected for testing in Example 2.

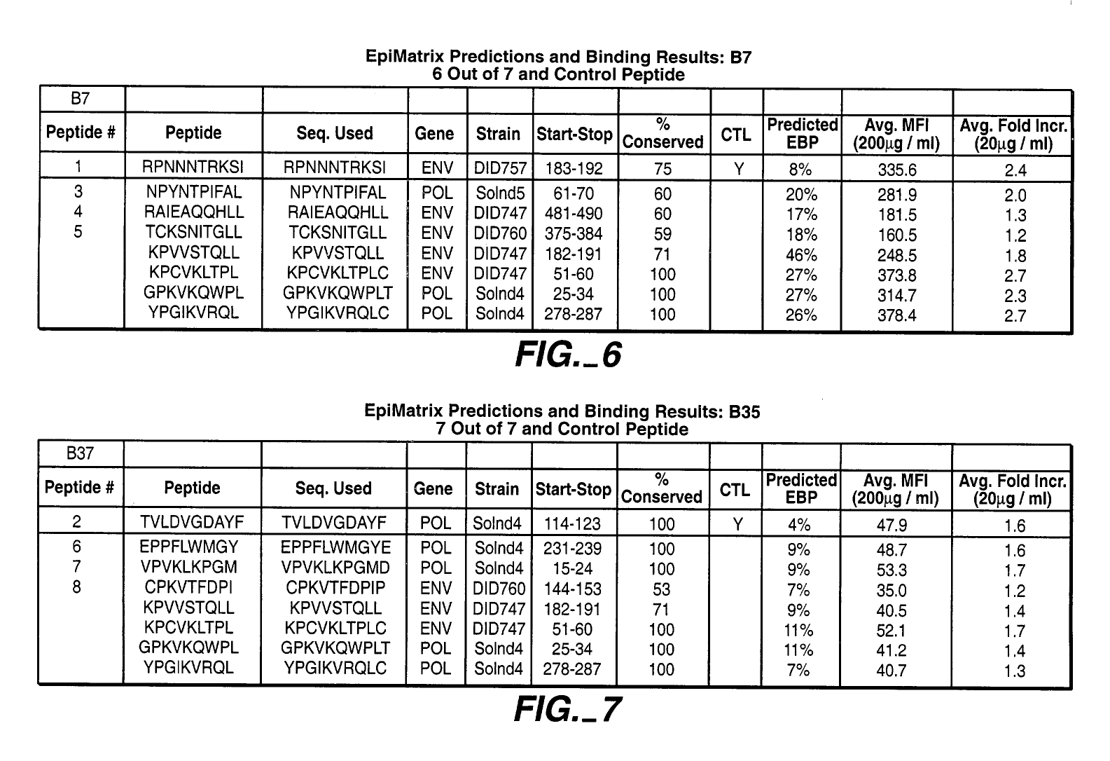

FIGS. 6-9 are table illustrations containing the EpiMatrix predictions and binding results for the B7 (FIG. 6), B37 (FIG. 7), A2 (FIG. 8) and A11 (FIG. 9) alleles tested in Example 2.

FIG. 10 is an illustration summarizing the steps of the T2 peptide binding assay.

FIG. 11 is a bar graph illustration showing the clustering of putative MHC ligands in the envelope protein of HIV (“env”). The number and location of putative ligands discovered to be (1) conserved across clades and (2) likely to bind to at least one human class I MHC in a “consensus” sequence obtained from the Los Alamos HIV Sequence Database is illustrated.

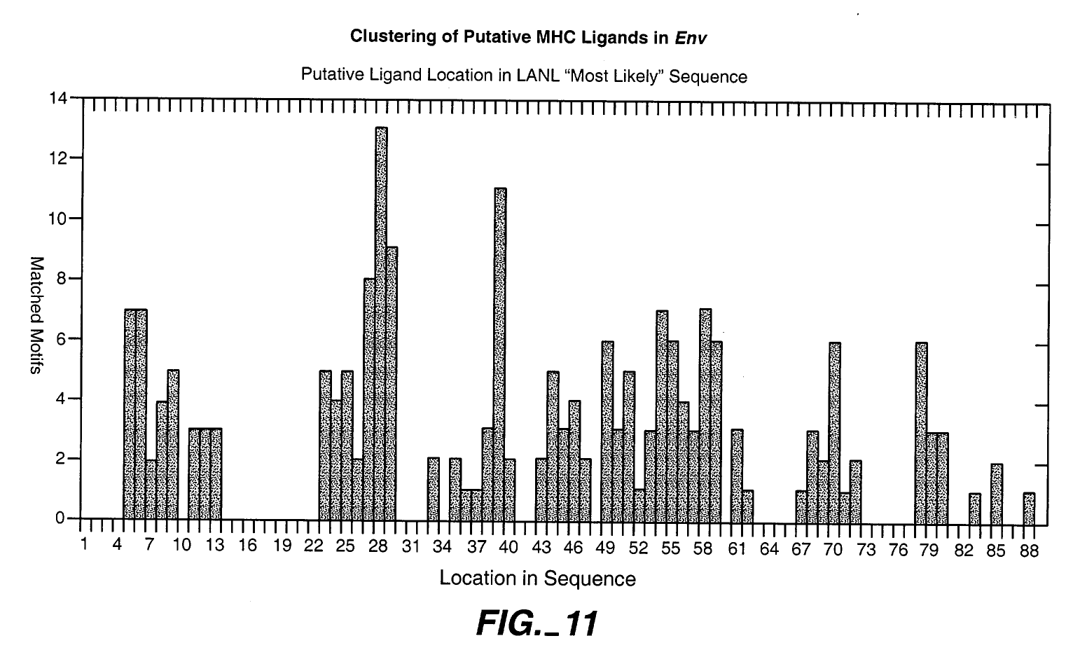

FIG. 12 is a illustration summarizing the results in Example 3 below.

### DETAILED DESCRIPTION OF THE INVENTION

A. Peptides, Polynucleotides and Antibodies

In one aspect, the invention provides cross-clade candidate peptides not heretofore recognized or known in the art. By “cross-clade” we mean able to elicit an effective immune response to infection or challenge by HIV isolates belonging to more than one HIV lade or subtype; i.e., at least two different isolates from different clades. These peptides were identified originally by screening an extensive database of HIV-1 sequences for strings of amino acids (peptides) that were conserved in many of these isolates and usually in more than one dade using Conservatrix, a computer based sequence matching and counting tool. Conservatrix compares the sequence of every 10 amino aid long peptide in the sequence database for identity with every other 10 amino acid sequence. The program was configured to search for peptides based on absolute conservation, i.e., no amino acid substitutions at any position or, in other words, complete identity. The conserved peptides were then evaluated for potential to bind to HLA molecules of the MHC, and those that were likely to bind to one or more HLA molecule were selected. EpiMatrix, an epitope search algorithm was employed to carry out this function and to score the conserved ligands. The EpiMatrix method for scoring peptides has been described. De Groot, AIDS Research and Human Retroviruses 7:139-42 (1997).

These peptide sequences are characterized by:

(i) comprising between eight and fifty amino acids;

(ii) having complete sequence identity with an HIV-1 amino acid sequence that is absolutely conserved across at least 2 strains of HIV;

(iii) having the ability to bind to a human HLA molecule based on possession of amino acid patterns that conform to a MHC binding matrix motif for a human HLA molecule of the MHC; and

(iv) having the ability to bind to a human HLA molecule in the T2 in vitro peptide binding assay, as demonstrated by exhibition of greater than 1.3-fold increase in MFI (mean fold increase) upon FACS (fluorescence-activated cell sorter) analysis.

(v) having the ability to activate T cells from HIV positive patients in at least one in vitro assay selected from the group consisting of the ELIspot T cell assay, the ELIspot T cell restimulation assay, T cell proliferation assays, intracellular cytokine staining assays, the Brefeldin incorporation assay and tetramer staining technique.

A human MHC binding matrix motif for a human MHC allele is a quantitative estimation of the relative ability of an amino acid in a given sequence to non-covalently bind to another amino acid. Such motifs are generally derived from lists of peptides known to bind to a given HLA molecule and are restricted by the corresponding MHC allele, as described later in the specification.

More specifically, the peptide sequences are characterized as having between eight and twenty-five amino acids, preferably between eight and eleven amino acids, most preferably between nine and ten amino acids. The peptides can be any size between the specified minimums and maximums independently; for example, one cross-clade candidate peptide may comprise eight amino acids and another may comprise eleven or fifteen amino acids.

Even more specifically, the HIV cross-clade candidate peptides exhibit complete sequence identity with any of the partial amino acid sequences of HIV-1 proteins, for example, with an amino acid sequence of the env, pol, nef, rev, vif, vpu, vpx, vpr or tat protein, and the binding matrix motif to which they bind is an HLA-A2 or an HLA-B7 motif.

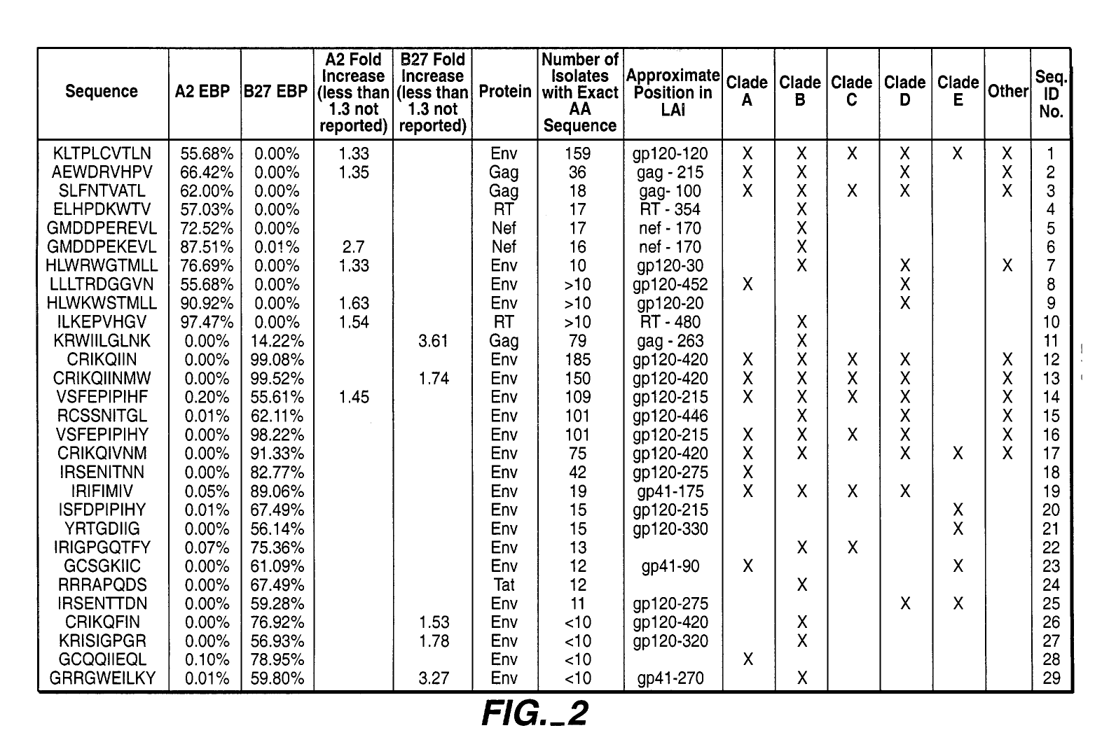

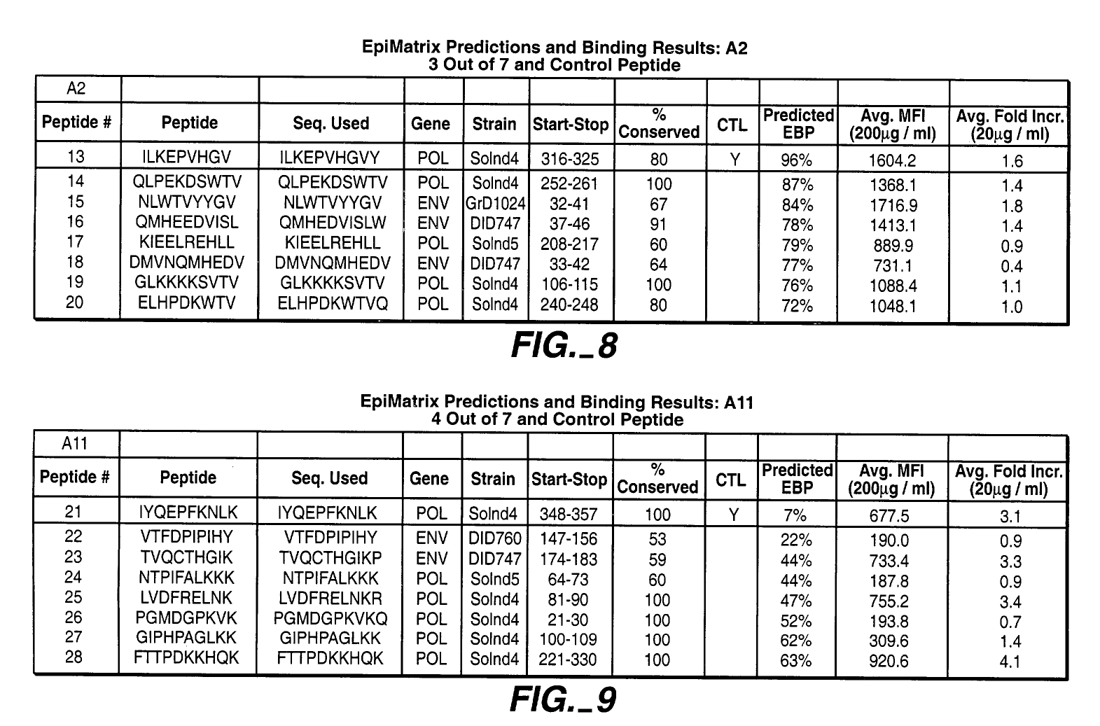

Most specifically, the HIV cross-clade candidate peptides comprise sequences corresponding to the HIV peptides shown in any of FIG. 2 (SEQ ID NO:1-27), TABLES 6-31 (SEQ ID NO: 28-626); and FIGS. 6-9 and TABLE 1-4 (SEQ ID NO:627-672). Such sequences may correspond to a consensus sequence obtained from the Los Alamos HIV Sequence Database and/or from the HIV-1 Seqeunce Database in Genbank.

The cross-clade candidate peptides can be produced by well known chemical procedures, such as solution or solid-phase peptide synthesis, or semi-synthesis in solution beginning with protein fragments coupled through conventional solution methods, as described by Dugas & Penney, Bioorganic Chemistry, 54-92 (Springer-Verlag, New York, 1981). For example, peptides can be synthesized by solid-phase methodology utilizing an PE-Applied Biosystems 430A peptide synthesizer (commercially available from Applied Biosystems, Foster City, Calif.) and synthesis cycles supplied by Applied Biosystems. Boc amino acids and other reagents are commercially available from PE-Applied Biosystems and other chemical supply houses. Sequential Boc chemistry using double couple protocols are applied to the starting p-methyl benzhydryl amine resins for the production of C-terminal carboxamides. After synthesis and cleavage, purification is accomplished by reverse-phase C18 chromatography (Vydac) column in 0.1% TFA with a gradient of increasing acetonitrile concentration. The solid phase synthesis could also be accomplished using the FMOC strategy and a TFA/scavenger cleavage mixture. Peptides may also be prepared by 9-fluoronylmethoxycarbonyl (Fmoc) synthesis on an automated synthesizer, for example, on a Rainen Symphony/Protein Technologies synthesizer (Synpep, Dublin, Calif.).

When produced by conventional recombinant means, the cross-clade candidate peptide can be isolated either from the cellular contents by conventional lysis techniques or from cell medium by conventional methods, such as chromatography (see, e.g., Sambrook et al., Molecular Cloning. A Laboratory Manual., 2d Edition (Cold Spring Harbor Laboratory, N.Y. (1989). The general construction and use of synthetic HIV peptides is disclosed in U.S. Pat. Nos. 5,817,318 and 5,876,731, the contents of which are incorporated by reference.

The cross-clade candidate peptide can be encoded by synthetic or recombinant polynucleotides, including peptides fused to carrier proteins. In another aspect, the invention includes such polynucleotides encoding the cross-clade candidate peptides. The polynucleotide can be a recombinant construct, such as a vector or plasmid, that contains the polynucleotide encoding the cross-clade candidate peptide or fusion protein under the operative control of polynucleotides encoding regulatory elements such as promoters, termination signals, and the like. “Operatively linked” means that the components so described are in a relationship permitting them to function in their intended manner. For example, a control sequence operatively linked to a coding sequence is ligated such that expression of the coding sequence is achieved under conditions compatible with the control sequence. “Control sequence” means a polynucleotide sequence that is necessary to effect the expression of coding and non-coding sequences to which they are ligated. Control sequences are well known in the art and generally include promoter, ribosomal binding site, and transcription termination sequence. In addition, “control sequence” includes sequences which control the processing of the peptide encoded within the coding sequence. Such control sequences may include, without limitation, sequences controlling secretion, protease cleavage, and glycosylation of the peptide. The term “control sequences” is intended to include, at a minimum, components whose presence can influence expression, and it optionally can include additional components whose presence is advantageous, for example, leader sequences and fusion partner sequences. A “coding sequence” is a polynucleotide sequence that is transcribed and translated into a polypeptide. Two coding polynucleotides are “operably linked” if the linkage results in a continuously translatable sequence without alteration or interruption of the triplet reading frame. A polynucleotide is operably linked to a gene expression element if the linkage results in the proper function of that gene expression element to result in expression of the cross-clade candidate coding sequence. “Transformation” is the insertion of an exogenous polynucleotide (i.e., a “transgene”) into a host cell. The exogenous polynucleotide is integrated within the host genome. A polynucleotide is “capable of expressing” a cross-clade candidate peptide if it contains nucleotide sequences which contain transcriptional and translational regulatory information and such sequences are “operably linked” to polynucleotide which encode the cross-clade candidate peptide. A polynucleotide that encodes a peptide coding region can be then amplified, for example, by preparation in a bacterial vector, according to conventional methods, for example, described in the standard work Sambrook et al., Molecular Cloning: A Laboratory Manual (Cold Spring Harbor Press 1989). Expression vehicles include plasmids or other vectors. Prokaryotic vectors known in the art include plasmids such as those capable of replication in E. coli (such as, for example, pBR322, ColE1, pSC101, pACYC184, πV.X.).

The polynucleotide encoding the cross-clade candidate peptide can be prepared by chemical synthesis methods or by recombinant techniques. The polypeptides can be prepared conventionally by chemical synthesis techniques, such as those described by Merrifield, 85 J. Amer. Chem. Soc. 2149-2154 (1963). See also, Stemmer et al, 164 Gene 49 (1995). Synthetic genes, the in vitro or in vivo transcription and translation of which will result in the production of the protein, can be constructed by techniques well known in the art. See for example Brown et al., 68 Methods in Enzymology 109-151 (1979). The coding polynucleotide can be generated using conventional DNA synthesizing apparatus such as the Applied Biosystems Model 380A or 380B DNA synthesizers (commercially available from Applied Biosystems, Inc., 850 Lincoln Center Drive, Foster City, Calif. 94404).

The cross-clade candidate peptides can be expressed singly, or in a “string of beads” format. In the latter case, the peptides are linked to one another by small, nonsense, amino acids sequences that function as spacers, for example three to ten alanine residues.

Alternatively, systems for cloning and expressing the cross-clade candidate peptides may comprise various microorganisms and cells well known in the recombinant technology art. These include, for example, various strains of E. coli, Bacillus, Streptomyces, Saccharomyces, as well as mammalian, yeast and insect cells. Suitable vectors are known and available from private and public laboratories and depositories and from commercial vendors. See for example, Sambrook et al., Molecular Cloning: A Laboratory Manual (Cold Spring Harbor Press 1989); and PCT Patent Publication WO 94/01139. These vectors permit the transfer of the polynucleotides into the patient's target cells and expression of the synthetic gene sequence in vivo, or expression of it as a peptide or fusion protein in vitro.

Polynucleotide gene expression elements useful for the expression of cDNA encoding peptides include, but are not limited to (a) viral transcription promoters and their enhancer elements, such as the SV40 early promoter, Rous sarcoma virus LTR, and Moloney murine leukemia virus LTR; (b) splice regions and polyadenylation sites such as those derived from the SV40 late region; and (c) polyadenylation sites such as in SV40. Recipient cells capable of expressing the cross-clade candidate peptides are transfected and used as host cells. The transfected recipient cells are cultured under conditions that permit expression of the cross-clade candidate peptides, which are recovered from the culture. Mammalian cells, such as Chinese Hamster ovary cells (CHO) or COS-1 cells, can be used as host cells. These host cells can be used in connection with poxvirus vectors, such as vaccinia or swinepox. Suitable non-pathogenic viruses can be engineered to carry the synthetic gene into the cells of the host include poxviruses, such as vaccinia, adenovirus, retroviruses and the like. A number of such non-pathogenic viruses are commonly used for human gene therapy, and as carriers for other vaccine agents, and are known and selectable by one of skill in the art. The selection of other suitable host cells and methods for transformation, culture, amplification, screening and product production and purification can be performed by one of skill in the art by reference to known techniques, see, e.g., Gething & Sambrook, 293 Nature 620-625 (1981). Yet another system that can be employed is the baculovirus expression system and vectors. Such systems are well known in the art. See, e.g., Lucklow & Summers, 17 Virology 31 (1989) and Miller, 42 Ann Rev Microbiol. 177 (1988).

General construction and use of polynucleotides encoding for non-infectious, replication-defective, self-assembling HIV-1 viral particles containing HIV antigenic markers is disclosed in U.S. Pat. No. 5,866,320, the contents of which are incorporated by reference.

Polynucleotides encoding the cross-clade candidate peptides can be used in a variety of ways. For example, a polynucleotide can express the cross-clade candidate peptide in vitro in a host cell culture. After suitable purification, the expressed cross-clade candidate peptide can be incorporated into a pharmaceutical reagent, immunogenic composition and/or vaccine as described more fully below. Alternatively, the polynucleotide encoding the cross-clade candidate peptide can be administered directly into a human patient as “naked DNA”. See Cohen, 259 Science 1691-1692 (1993); Fynan et al., 90 Proc. Natl. Acad. Sci. USA, 11478-82 (1993); and Wolff et al., 11 BioTechniques 474-485 (1991). This results in expression of the cross-clade candidate peptide by the patient's host cells and subsequent presentation to the immune system to induce anti-candidate epitope T cell responses (T helper cells and cytotoxic T cells) and also HIV antibody formation in vivo.

Determination of the sequence of the polynucleotide coding region that codes for the cross-clade candidate peptide can be performed using commercially available computer programs, such as DNA Strider and Wisconsin GCG. Owing to the natural degeneracy of the genetic code, the skilled artisan will recognize that a sizable yet definite number of DNA sequences can be constructed which encode the claimed peptides. See, Watson et al., Molecular Biology of the Gene, 436-437 (the Benjamin/Cummings Publishing Co. 1987).

Antibodies directed against a cross-clade candidate peptide are yet another aspect of this invention. Polyclonal antibodies are produced by immunizing a mammal with a peptide immunogen. Suitable mammals include primates, such as monkeys; smaller laboratory animals, such as rabbits and mice, as well as larger animals, such as horse, sheep, and cows. Such antibodies can also be produced in transgenic animals. However, a desirable host for raising polyclonal antibodies to a composition of this invention includes humans. The polyclonal antibodies raised are isolated and purified from the plasma or serum of the immunized mammal by conventional techniques. Conventional harvesting techniques can include plasmapheresis, among others. Such polyclonal antibodies can themselves be employed as pharmaceutical compositions of this invention. Alternatively, other forms of antibodies can be developed using conventional techniques, including monoclonal antibodies, chimeric antibodies, humanized antibodies and fully human antibodies. See, e.g., U.S. Pat. No. 4,376,110; Ausubel et al., Current Protocols in Molecular Biology (Greene Publishing Assoc. and Wiley Interscience, N.Y., 1992); Harlow & Lane, Antibodies: a Laboratory Manual, (Cold Spring Harbor Laboratory, 1988); Queen et al., 86 Proc. Nat'l. Acad. Sci. USA 10029-10032 (1989); Hodgson et al., 9 Bio/Technology 421 (1991); and PCT Patent Publications WO 92/04381 and WO 93/20210. Other antibodies can be developed by screening hybridomas or combinatorial libraries, or antibody phage displays (see Huse et al., 246 Science 1275-1281 (1988) using the polyclonal or monoclonal antibodies produced according to this invention and the amino acid sequences of the primary or optional immunogens.

The term “antibody” includes polyclonal antibodies, monoclonal antibodies (mAbs), chimeric antibodies, anti-idiotypic (anti-Id) antibodies to antibodies that can be labeled in soluble or bound form, and fragments, regions or derivatives thereof, regardless of how isolated or made. An “antigen binding region” is that portion of an antibody molecule which contains the amino acid residues that interact with an antigen and confer on the antibody its specificity and affinity for the antigen. This region includes the framework amino acid residues necessary to maintain the proper conformation of the antigen-binding residues.

B. Utility: Antigens and Immunogenic Compositions

The cross-clade candidate peptides of the invention, when introduced into cells as peptides, as components of a pseudo protein, or as oligonucleotides in a DNA vaccine or vectored vaccine, can be used to induce T cell responses in the vaccinated hosts. The T cell responses serve to improve the host's ability to contain infection either during or after challenge by HIV.

The cross-clade candidate peptides of the invention are useful as antigens for raising anti-HIV immune responses, such as T cell responses (cytotoxic T cells or T helper cells). An “antigen” is a molecule or a portion of a molecule (typically a foreign peptide) capable of stimulating an immune response, i.e., capable of inducing an animal (including a human) to produce antibody capable of binding to an epitope of that antigen. An “epitope” is that portion of an antigen molecule capable of being bound by a MHC molecule or protein and recognized by a T cell, or capable of being bound by an antibody. An antigen can have one or more than one epitope. An antigen is “immunologically reactive” in a highly selective manner, with its corresponding MHC protein or with antibody, and not with the multitude of other MHC proteins and antibodies present in the animal, which can be evoked by other antigens.

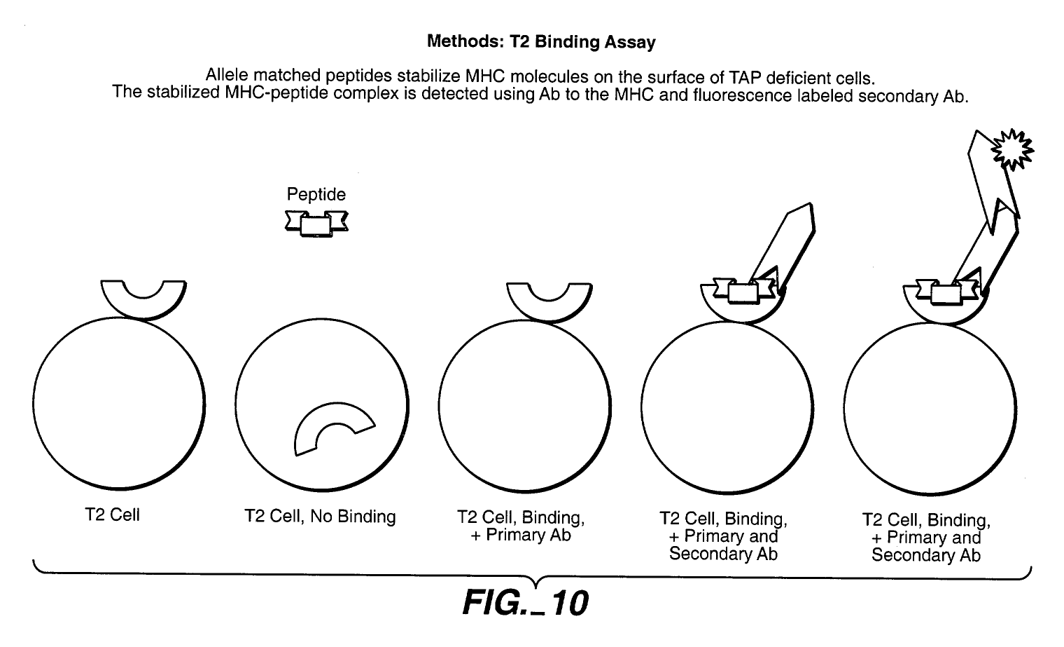

An antigen or foreign peptide is “immunologically reactive” with an T cell or with an antibody if it non-covalently binds to an MHC protein and is recognized by a T cell, or if it binds to an antibody. Immunological reactivity can be determined (1) by measuring T cell response in vitro (2) by measuring the kinetics of antibody binding, or (3) by assessing competition in binding using as competitors a known peptides containing an epitope against which the antibody or T cell response is directed. Such techniques are well known in the art. Peptides identified as immunologically reactive in the foregoing tests can be screened for efficacy by in vitro and in vivo assays. Such assays include immunization of an animal, e.g., a rabbit or a primate, with the peptide and evaluation of titers antibody to HIV-1 or to synthetic detector peptides corresponding to variant HIV sequences. Assays evaluating antibody titer in animals are well known in the art. See Example 3 and FIG. 10. Methods of determining spatial conformation of amino acids to predict non-covalent binding potential are known in the art also and include, for example, x-ray crystallography and 2-dimensional nuclear magnetic resonance.

The cross-clade candidate peptides can be employed in methods for reducing the viral levels of HIV-1. Such methods involve exposing a human to a cross-clade candidate peptide, actively inducing antibodies or cellular immune responses against HIV-1, and impairing the multiplication of the virus in vivo. This method is appropriate for an HIV-1 infected subject with a competent immune system, or an uninfected or recently infected subject. The method induces T cells and/or antibodies or cellular immune responses that react with HIV-1 and actively induces T cells that respond to HIV-1, which T cells and antibodies serve to reduce viral multiplication during any initial acute infection with HIV-1 and minimizes chronic viremia leading to AIDS. This method also lowers chronic viral multiplication in infected subjects, minimizing progression to AIDS. In other words, in already infected patients, this method of reduction of viral levels can reduce chronic viremia and progression to AIDS. In uninfected humans, this administration of the peptides of the invention can reduce acute and thus minimize chronic viremia leading to progression to AIDS. Treating, and “treatment” mean obtaining a desired pharmacologic or physiologic effect. The effect can be prophylactic in terms of completely or partially preventing a disorder or sign or symptom thereof, or can be therapeutic in terms of a partial or complete cure for a disorder and/or adverse effect attributable to the disorder. “Treating” and “treatment” also mean preventing a disorder from occurring in a subject that can be predisposed to a disorder, but has not yet been diagnosed as having it; inhibiting the disorder, i.e., arresting its development; or relieving or ameliorating the disorder. Among such patients suitable for treatment with this method are HIV-1 infected patients who are immunocompromised by disease and unable to mount a strong immune response. In later stages of HIV infection, the likelihood of generating effective titers of antibodies is less, due to the immune impairment associated with the disease. Also among such patients are HIV-1 infected pregnant women, neonates of infected mothers, and unimmunized patients with putative exposure (e.g., a human who has been inadvertently “stuck” with a needle used by an HIV-1 infected human).

An “effective amount” or “therapeutically or immunologically effective amount” is an amount sufficient to obtain the desired physiological effect, e.g., treatment of HIV. An effective amount of the cross-clade candidate peptide or vector expressing a cross-clade candidate peptide is typically determined by the physician taking account of the factors normally considered to determine appropriate dosages, including the age, sex, and weight of the subject to be treated, the condition being treated, and the severity of the condition.

C. Modes and Methods and of Administration and Ingredients

The cross-clade candidate peptides of the invention can be administered orally, topically, parenterally e.g. subcutaneously, intraperitoneally, by viral infection, or intravascularly. Depending upon the manner of introduction, the cross-clade candidate peptides can be formulated in a variety of ways. The concentration of Cross-clade candidate peptides in the formulation can vary from about 0.1-100 wt. %.

The amount of the cross-clade candidate peptide or polynucleotides of the invention present in each vaccine dose is selected with regard to consideration of the patient's age, weight, sex, general physical condition and the like. The amount of cross-clade candidate peptide required to induce an immune response, preferably a protective response, or produce an exogenous effect in the patient without significant adverse side effects varies depending upon the pharmaceutical composition employed and the optional presence of an adjuvant. Generally, for the compositions containing cross-clade candidate peptide, each dose will comprise between about 50 μg to about 1 mg of the cross-clade candidate peptide per ml of a sterile solution. A more preferred dosage can be about 200 μg of cross-clade candidate peptide. Other dosage ranges can also be contemplated by one of skill in the art. Initial doses can be optionally followed by repeated boosts, where desirable. The method can involve chronically administering the cross-clade candidate peptide composition. For therapeutic or prophylactic use, repeated dosages of the immunizing compositions can be desirable, such as a yearly booster or a booster at other intervals. The dosage administered will, of course, vary depending upon known factors such as the pharmacodynamic characteristics of the particular agent, and its mode and route of administration; age, health, and weight of the recipient; nature and extent of symptoms, kind of concurrent treatment, frequency of treatment, and the effect desired. Usually a daily dosage of active ingredient can be about 0.01 to 100 mg/kg of body weight. Ordinarily 1.0 to 5, and preferably 1 to 10 mg/kg/day given in divided doses 1 to 6 times a day or in sustained release form is effective to obtain desired results.

The cross-clade candidate peptide can be employed in chronic treatments for subjects at risk of acute infection due to needle sticks or maternal infection. A dosage frequency for such “acute” infections may range from daily dosages to once or twice a week i.v. or i.m., for a duration of about 6 weeks. The peptides can also be employed in chronic treatments for infected patients, or patients with advanced HIV. In infected patients, the frequency of chronic administration can range from daily dosages to once or twice a week i.v. or i.m., and may depend upon the half-life of the immunogen (e.g., about 7-21 days). However, the duration of chronic treatment for such infected patients is anticipated to be an indefinite, but prolonged period.

For such therapeutic uses, the cross-clade candidate peptide formulations and modes of administration are substantially identical to the prophylactic formulations and modes of administration. They can be administered concurrently or simultaneously with other conventional therapeutics for HIV viral infection.

The cross-clade candidate peptides can be administered either as individual therapeutic agents or in combination with other therapeutic agents. Cross-clade candidate peptides can be administered alone, but are generally administered with a pharmaceutical carrier selected on the basis of the chosen route of administration and standard pharmaceutical practice. The vaccine can further comprise suitable, i.e., physiologically acceptable, carriers--preferably for the preparation of injection solutions—and further additives as usually applied in the art (stabilizers, preservatives, etc.), as well as additional drugs. The patients can be administered a dose of approximately 1 to 10 μg/kg body weight, preferably by intravenous injection once a day. For less threatening cases or long-lasting therapies the dose can be lowered to 0.5 to 5 μg/kg body weight per day. The treatment can be repeated in periodic intervals, e.g., two to three times per day, or in daily or weekly intervals, depending on the status of HIV-1 infection or the estimated threat of an individual of getting HIV infected.

For parenteral administration, peptides of the invention can be formulated as a solution, suspension, emulsion or lyophilized powder in association with a pharmaceutically acceptable parenteral vehicle. Examples of such vehicles are water, saline, Ringer's solution, dextrose solution, and 5% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation is sterilized by commonly used techniques. Suitable pharmaceutical carriers are described in the most recent edition of Remington's Pharmaceutical Sciences, a standard reference text in this field of art. For example, a parenteral composition suitable for administration by injection is prepared by dissolving 1.5% by weight of active ingredient in 0.9% sodium chloride solution. The preparation of these pharmaceutically acceptable compositions, having appropriate pH isotonicity, stability and other conventional characteristics is within the skill of the art. Suitable pharmaceutically acceptable carriers for use in an immunogenic composition are well known to those of skill in the art. Such carriers include, for example, saline, a selected adjuvant, such as aqueous suspensions of aluminum and magnesium hydroxides, liposomes, oil in water emulsions, and others.

The vaccine or immunogenic composition can include as the active ingredient one of the following components: (a) a cross-clade candidate peptide, alone or combined with a carrier protein conjugate; (b) a polynucleotide encoding a cross-clade candidate; (c) a recombinant virus carrying the synthetic gene or molecule; or (d) a bacteria carrying the cross-clade candidate peptide. The selected active component is present in a pharmaceutically acceptable carrier, and the composition can contain additional ingredients. Formulations containing the cross-clade candidate peptide can contain other active agents, such as adjuvants and immunostimulatory cytokines, such as IL-12 and other well-known cytokines, for the peptide compositions. The CpG (cytosine-guanine dinucleotide) formulations of immunostimulatory DNA (Coley Pharmaceuticals) are another exemplary adjuvant.

Cross-clade candidate peptide can be linked to a suitable carrier in order to improve the efficacy of antigen presentation to the immune system. Such carriers can be, for instance, organic polymers. A carrier protein can enhance the immunogenicity of the peptide immunogen. Such a carrier can be a larger molecule that has an adjuvant effect. Exemplary conventional protein carriers include, keyhole limpet hemocyan, E. coli DnaK protein, galactokinase (galK, which catalyzes the first step of galactose metabolism in bacteria), ubiquitin, α-mating factor, β-galactosidase, and influenza NS-1 protein. Toxoids (i.e., the sequence which encodes the naturally occurring toxin, with sufficient modifications to eliminate its toxic activity) such as diphtheria toxoid and tetanus toxoid can also be employed as carriers. Similarly a variety of bacterial heat shock proteins, e.g., mycobacterial hsp-70 can be used. Glutathione reductase (GST) is another useful carrier. One of skill in the art can readily select an appropriate carrier.

Viruses can be modified by recombinant DNA technology such as, e.g. rhinovirus, poliovirus, vaccinia, or influenzavirus, etc. The peptide can be linked to a modified, i.e., attenuated or recombinant virus such as modified influenza virus or modified hepatitis B virus or to parts of a virus, e.g., to a viral glycoprotein such as, e.g., hemagglutinin of influenza virus or surface antigen of hepatitis B virus, in order to increase the immunological response against HIV-1 viruses and/or infected cells. The cross-clade candidate peptides can comprise fusion proteins, in which they are linked to a suitable carrier such as a recombinant or attenuated virus or a part of a virus. Exemplary are influenza virus hemagglutinin, hepatitis B virus surface antigen, surface proteins of rhinovirus, poliovirus, sindbis virus, coxsackievirus, etc.

Alternatively, the polynucleotides encoding the cross-clade candidate peptides of the invention can be designed for direct administration as “naked DNA”. Suitable vehicles for direct DNA, plasmid polynucleotide, or recombinant vector administration include, without limitation, saline, or sucrose, protamine, polybrene, polylysine, polycations, proteins, calcium phosphate, or spermidine. See e.g, PCT International patent application WO 94/01139. As with the immunogenic compositions, the amounts of components in the DNA and vector compositions and the mode of administration, e.g., injection or intranasal, can be selected and adjusted by one of skill in the art. Generally, each dose will comprise between about 50 μg to about 1 mg of immunogen-encoding DNA per ml of a sterile solution.

For recombinant viruses containing the coding polynucleotide, the doses can range from about 20 to about 50 ml of saline solution containing concentrations of from about 1×10⁷ to 1×10¹⁰ pfu/ml recombinant virus of the invention. One human dosage is about 20 ml saline solution at the above concentrations. However, it is understood that one of skill in the art can alter such dosages depending upon the identity of the recombinant virus and the make-up of the immunogen that it is delivering to the host.

The amounts of the commensal bacteria carrying the synthetic gene or molecules to be delivered to the patient will generally range between about 10³ to about 10¹² cells/kg. These dosages, will of course, be altered by one of skill in the art depending upon the bacterium being used and the particular composition containing immunogens being delivered by the live bacterium.

Aspects of the invention may be implemented in hardware or software, or a combination of both. However, preferably, the algorithms and processes of the invention are implemented in one or more computer programs executing on programmable computers each comprising at least one processor, at least one data storage system (including volatile and non-volatile memory and/or storage elements), at least one input device, and at least one output device. Program code is applied to input data to perform the functions described herein and generate output information. The output information is applied to one or more output devices, in known fashion.

Each program may be implemented in any desired computer language (including machine, assembly, high level procedural, or object oriented programming languages) to communicate with a computer system. In any case, the language may be a compiled or interpreted language.

Each such computer program is preferably stored on a storage media or device (e.g., ROM, CD-ROM, tape, or magnetic diskette) readable by a general or special purpose programmable computer, for configuring and operating the computer when the storage media or device is read by the computer to perform the procedures,described herein. The inventive system may also be considered to be implemented as a computer-readable storage medium, configured with a computer program, where the storage medium so configured causes a computer to operate in a specific and predefined manner to perform the functions described here.

The details of one or more embodiments of the invention are set forth in the accompanying description. Although any methods and materials similar or equivalent to those described herein can be used in the practice or testing of the invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference. The following examples are presented in order to more fully illustrate the preferred embodiments of the invention. These examples should in no way be construed as limiting the scope of the invention, as defined by the appended claims.

### EXAMPLE 1

Prediction of Well-conserved HIV-1 Ligands Using a Matrix-based Algorithm, EpiMatrix

Introduction. This Example discloses a prospective design of multivalent HIV immunogens tailored to reflect the diversity of HIV isolates and to promote cross-clade protection in settings where more than one HIV strain and more than one HIV lade is being transmitted. It has been speculated that EpiMatrix and other computer-driven algorithms predict putative MHC ligands and CTL epitopes can be employed in the prospective drug design. See for example, Davenport et al., 42 Immunogenetics 392-7 (1995); Hammer et al., 180 J. Exp. Med. 2353-8 (1994); Flackenstein et al., 240 Eur. J. Biochem. 71-7 (1996). This Example investigates the efficacy of using EpiMatrix, a matrix-based algorithm for T-cell epitope prediction, to identify conserved Class I-restricted MHC ligands and potential CTL epitopes.

Background. This prospectively designed HIV-1 vaccine is based on the central role of CTL in the host immune response to HIV-1. First, HIV-1 peptides that bind to the host MHC molecules or proteins (i.e., ligands) are identified. Recognition of such MHC ligands by CTL cells is dependent on the presentation of the antigen to the T cell (via the T cell epitope) by MHC molecules. Peptides presented to T cells by Class I MHC molecules are derived from foreign or self-protein antigens that have been processed in the cytoplasm. The peptides non-covalently bind to MHC molecules in a linear fashion; the binding is determined by the interaction of the peptide's amino acid side-chains with binding pockets in the MHC molecule. Binding of peptides to MHC molecules is constrained by the nature of the side-chains; only selected peptides will fit the constraints of any given MHC molecule's binding pockets.

The characteristics of peptides likely to bind to a given MHC molecule or protein can be directly deduced from pooled sequencing data (from peptides bulk-eluted off MHC molecules) in MHC binding peptide libraries. We have developed a method to describe the relative promotion or relative inhibition of binding afforded by each position in a peptide to the MHC of interest. The EpiMatrix algorithm is a computer-based program, which carries out this method, as described below.

EpiMatrix ranks all 10 amino acid long segments from any protein sequence by estimated probability of binding to a given MHC ligand by comparing the segments to a matrix. This estimated binding probability (EBP) is derived by comparing the EpiMatrix score for the given test segment to those of known sequences that bind (“binders”) and to sequences presumed not to bind (“non-binders”). Retrospective studies have demonstrated that EpiMatrix accurately predicts MHC ligands. See DeGroot et al., AIDS Research and Human Retroviruses 7:139-42 (1997); Jesdale et al., in Vaccines '97. (Cold Spring Harbor Press, Cold Spring Harbor, 1997).

In this Example, we used the EpiMatrix algorithm to examine the sequences of HIV-1 strains published in the 1995 version of the Los Alamos National Laboratory HIV Sequence database. We identified conserved sequences in the published strains and examined these for their potential to bind to one of two known MHC proteins, the A2 allele and the B27 allele. Those sequences having adequate binding potential were then tested for actual binding to determine which, if any could be useful for HIV-1 vaccine development.

Generation of a MHC binding matrix motif. Various methods were used in the generation of MHC binding matrix motifs. Briefly, various independent sources of information on the relative promotion or inhibition of each amino acid in each position of the sequence are identified. For each source of information, an estimation of the relative promotion or inhibition of binding is quantified. In a generic sense, this quantification is based on a relative rate calculation: the rate of an amino acid in a given position relative to its median rate across all positions. The independent sources of information include, without limitation, known ligands (see Huczko et al., 151 J. Immunol. 2572 (1993)), pooled sequencing of naturally eluated peptides (see Kubo et al., 152 J. Immunol. 3913-24 (1993)), peptide side-chain scanning techniques (see Hammer et al., 180 J. Exp. Med. 2353-8 (1994)), and the identification of ligands with specific characteristics through random phage techniques (see Flackenstein et al., 240 Fur. J. Biochem. 71-7 (1996)). The quantified rates are matrixed and then combined in order to maximize the resultant matrix “motif's” ability to separate a list of known ligands from the other peptides contained within their original sequences. Specifically, the two matrix motifs based on single datasets with the best individual predictive power as assessed using the Kruskal-Wallis non-parametric test are first combined with each other. The best resultant of these two is then combined with the third most individually predictive and so on until all matrix “motifs” have been analyzed. The result of this process is then combined using the method of Parker et al., 152 J. Immunol. 163-75 (1994) to achieve a final predictive matrix motif for each MHC allele.

Generating an EpiMatrix score. Each putative MHC binding region within a given protein sequence is scored by assigning to it an estimate of the relative promotion or inhibition of binding for each amino acid, and summing these to create a summary score for the entire peptide. Higher EpiMatrix scores indicate greater MHC binding potential. After comparing the score to the scores of known MHC ligands, an “estimated binding probability” or EBP, is generated. The EBP represents the proportion of known ligand peptides with EpiMatrix scores as high or higher than the score obtained by the ligand in the Example.

EBP is derived from the EpiMatrix score by determining how many published ligands for the allele would earn that same score or a higher score (a measure of sensitivity). EBPs range from 100% (highly likely to bind) to less than 1% (very unlikely to bind). The majority of 9 and 10 mers in any given protein sequence fall below the 1% estimated binding probability for any given MHC binding matrix. See De Groot, et al., AIDS Research and Human Retroviruses 7:139-42 (1997).

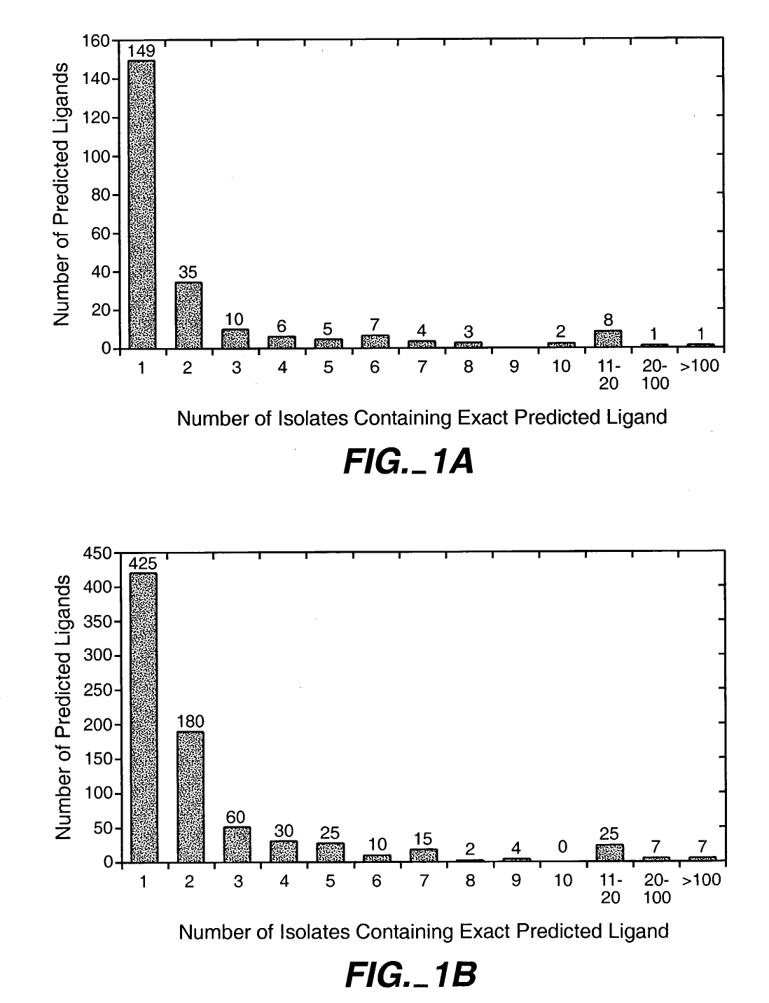

Selection of peptides. Each of the HIV-1 proteins was analyzed individually and independently. The analysis was carried out using the sequence of the HIV-1 isolate in the publicly available Los Alamos HIV sequence database (the “LANL” database). See Korber & Meyers, eds, HIV Sequence Database, Los Alamos HIV Database, 1995. (Los Alamos National Laboratories, New Mexico, 1995). Beginning with the first amino acid in the coding sequence, each HIV protein sequence was divided into strings of ten, consecutive amino acids each. Each string overlapped the preceding string by nine amino acids. Thus, for example, the first string constructed comprised amino acids 1-10 of the HIV-1 env amino acid sequence and the second string constructed comprised amino acids 2-11 of the HIV-1 env amino acid sequence, and so on. These 10-mer strings were then compared to the A2 and B27 MHC binding matrix motifs generated by the EpiMatrix algorithm version 1.0 to assess potential ability to bind as explained in detail above. Peptides that scored higher than 50% EBP were deemed putative ligands and selected for further analysis. Each of these putative ligands was compared to all other putative ligands using a spreadsheet and command macro that orders the strings from most common to unique. The results are illustrated generally in FIG. 1. Strings that were conserved in greatest number of HIV-1 isolates (the exact number depended on the number of isolates available in the LANL database) were selected for the next step in the analysis. Twenty-eight peptides were selected using this method. One of the 28 selected peptides selected corresponded to a published CTL epitope, and was chosen to serve as a control. An additional peptide that was selected to serve as a positive control as for this study, KRWIILGLNK, scored lower that 50% on the B27 EBP matrix. However, it was chosen because it was the only available HIV-1 B27 ligand that had been fine-mapped.

The T2 in vitro peptide binding assay was performed on each of the 28 peptides following the method described in Nijman et al., 23 Eur. J. Immunol. 1215-9 (1993) and as follows. This assay relies on the ability of exogenously added peptides to stabilize the Class I/β2 microglobulin structure on the surface of TAP-defective cell lines. For these assays, we used the antigen processing mutant cell line T2, transfected with the HLA B27 gene (T2/B27). The transfected cells were cultured in Iscove Modified Dulbecco's Medium (IMDM), 10% fetal bovine serum, and 20 μg/ml gentamycin. A monoclonal antibody to HLA-B27 produced by the MEI hybridoma (ATCC accession number 1-HB-119; see Ellis et al., 5 Hum. Immunol. 49-59 (1982)) was used to assess HLA-B27 expression at the cell surface as indicative of peptide binding and stabilization of the B27 molecule. A second monoclonal antibody produced by the BB7.2 hybridoma (ATCC accession number HB-82; see Parham & Brodsky, 3 Hum. Immunol. 277 99 (1981)) was used to assess HLA-A2 expression at the cell surface as indicative of peptide binding and stabilization of the A2 molecule.

Three hundred thousand cells in 100 μl of IMDM, 10% FBS, and 20 μg/ml gentamycin medium were incubated with no peptide, or 100 μl synthetic peptide solution overnight at 37° C., in an atmosphere of 5% CO₂. The T2 cell/peptide suspension was pelleted at 1000 rpm. the supernatant was discarded, and the suspension was stained with 100 μl of BB7.2, an HLA-A2 specific mouse monoclonal primary antibody (1 hr at 4° C.). Two wells per peptide did not receive the primary antibody, but only the PBS staining buffer. The cells were washed 3× with cold (4° C.) staining butter PBS, 0.5% FBS, 0.02% NaN₃, and stained for 30 min at 4° C. with 100 μl FITC-labeled goat anti-mouse immunoglobulin (Pharmingen, 12064-D). The cells were again washed three times and fixed in 1% paraformaldehyde. Fluorescence of viable T2 cells was measured at 488 nm on a FACScan flow cytometer (Becton-Dickinson, NJ).

For each of the 28 peptides, 12 wells were assayed. Wells containing each peptide at 0, 2, 20, and 200 μg/ml concentrations were assayed using primary antibody to the molecule to which the peptide is predicted to bind, using primary antibody to the molecule to which the peptide was not predicted to bind, and using no primary antibody.

Analysis and interpretation of binding assays. Peptide binding to MHC molecules stabilizes MHC expression at the cell surface, and can be measured by FACS sorting. Data produced by the FACS analysis is represented as the mean linear fluorescence (MLF) averaged over 10,000 events. As the criterion for positive binding, we used a cut-off of 1.3-fold greater MFI (mean fold increase) in any of the test peptide-containing three wells as compared to the control well (containing no peptide).

Results. Two of the 28 were previously published ligands. Ten peptides of the 28 peptides tested induced an increase in the MFI of 1.3-fold or greater in the T2 in vitro peptide binding assay. These results are illustrated in FIG. 2, columns 4 and 5. The published controls bound as expected. Peptides shown in FIG. 2 were selected for testing in part because they were predicted to bind to A2 and not to B27, or vice versa. Upon testing, this was confirmed because none of the peptides predicted to bind to A2 bound to B27 and vice versa.

Summary. New MHC ligands from human immunodeficiency virus type 1 (HIV-1) which are highly conserved across HIV-1 clades and which may serve to induce cross-reactive cytotoxic T lymphocytes (CTLs) were identified. EpiMatrix was used to predict putative ligands from HIV-1 for HLA-A2 and HLA-B27. Twenty-six peptides that were both likely to bind and highly conserved across HIV-1 strains in the Los Alamos HIV sequence database were selected for assessment of binding in the T2 stabilization assay. Two peptides that had previously been described as able to bind in the publicized literature, and which were also predicted to be highly likely to bind for A2 and B27 by EpiMatrix and conserved across HIV-1 strains were selected to serve as positive controls. Ten new MHC ligands were identified. The control peptides bound, as expected. These data confirm that EpiMatrix can be used to screen HIV-1 protein sequences for highly conserved sequences that are likely to bind to MHC and that may prove to be highly conserved HIV-1 CTL epitopes.

Conclusion. Rapid identification of MHC ligands, which can then be tested in T-cell assays, is desirable for HIV-1 vaccine development. Computer-driven analysis of HIV sequences permits prospective identification of such conserved CTL epitopes. Determination of peptides that bind to MHC molecules is the first step in the process of identifying T-cell epitopes. Identification of MHC ligands from primary HIV-1 sequences is particularly relevant for HIV vaccine development and immunopathogenesis research. Matrix-based motifs have been developed to improve on the specificity of anchor-based motifs. The advantage of matrix motifs is that peptides can be given a score that represents the sum of the potential for each ammo acid in the sequence to promote or inhibit binding.

Predicting regions or sequences of immunological interest is the first step to determining whether the region or sequence is likely to be recognized by primed T cells and to be defined as a CTL epitope. Likely regions or sequences must be tested and the prediction confirmed by binding assays to confirm the prediction. Immunogencity of the peptides must then be confirmed by measuring whether CTL recognize the peptide in standard T-cell assays.

Methods of analysis disclosed here permit the comparison of putative MHC ligands across HIV-1 clades and permit the weighting of predictions for the prevalence of HLA alleles in human populations. Utilization of these computer-driven methods enables the prospective identification of cross-clade (cross-reactive) and promiscuous epitopes, and puts development of a cross-clade HIV-1 vaccine within reach.

### EXAMPLE 2

### A Regional HIV Vaccine for India

Introduction. India has one of the highest burdens of HIV infection of any country in the world: 4.1 million individuals are believed infected and the rate of infection is expected to accelerate over the next decade. Because of the prevalence of selected HIV-1 clades on the Indian sub-continent and the unique genetic make-up (i.e., HLA distribution) of the Indian population, a region-specific HIV vaccine would be conceivable and advantageous.

We selected HIV peptides conserved across the HIV-1 strains that have been isolated to date in India. We evaluated these selected peptides for their projected binding capability to selected MHC Class I molecules, using the computer-driven modeling program, EpiMatrix, as more fully described in Example 1.

Analysis. Sixty six HIV-1 amino acid sequences from India (55 env, 6 gag and 5 pot sequences) were identified as having been isolated in India or isolated from individuals who acquired their HIV infection in India from a review and analysis of the published literature. The 66 amino acid sequences divided into strings of 10 mers overlapping by 9 amino acids as fully described in Example 1 and were examined for regions conserved in at least ˜50% (i.e., “highly conserved”) of the sequences. Twenty-eight sequences were found with conserved regions. The conserved sequences are illustrated in Tables 1-4 below. Twenty eight peptides were identified as (1) highly in the Indian HIV-1 sequences and (2) predicted to bind to the MHC Class I alleles HLA-A0201 [A2 in Table], HLA-A1101 [A11 in Table 4], HLA-B35, or HLA-B7 that are prevalent HLA alleles in India, as determined using EpiMatrix by comparing the sequences to the corresponding matrices.

These peptides were synthesized on a automated Rainen Symphony/Protein Technologies synthesizer (Synpep, Dublin, Calif.) using the 9-fluronylmethoxy-carbonyl (Fmoc) methodology according to the manufacture's protocol and tested in vitro using an MHC binding assay protocol following the methods of Ljunggren et al., Nature 346: 476-80 (1990); Nijman et al., Eur J Immunol 23:1215-19 (1993) and Brander et al., Clin Exp Immunol 101:107-13 (1995) and as detailed in Example 3 below. Fluorescence of viable T2 cells was measured on a FACScan flow cytometer (Becton-Dickinson, New Jersey). The data produced represented the mean linear fluorescence (MLF) of 10,000 events. Fluorescence data was analyzed using: (1) a two-factor ANOVA to determine treatment or plate effect, and (2) a multiple comparison to find significant differences between treatment means.

Results. Twenty out of the 28 predicted peptides (71%) stabilized the MHC Class I molecule for which they were predicted to bind. (p-values <0.001). The predictive accuracy of the B7 (86%) and B35 (100%) matrices for the EpiMatrix algorithm were slightly better in this Example than the predictive accuracy of the A11(42%) and A2(57%) matrices. B7 peptides predicted to also bind to B35 were able to stabilize B35 in vitro. B7 Peptides predicted to be unlikely to bind to B35 did not stabilize B35 in vitro. The reverse was also true; B35 peptides predicted to also bind B7 were able to stabilize B7 in vitro and B35 peptides predicted to be unlikely to bind to B7 did not stabilize B7 in vitro. The following TABLES correspond to FIGS. 6-9.

TABLE 1
B7

| Peptide # | Peptide | Seq. Mfg'd & Used | SEQ ID NO: |
|---|---|---|---|
| 1 | RPNNNTRKSI | RPNNNTRKSI | 627 |
| 3 | NPYNTPIFAL | NPYNTPIFAL | 628 |
| 4 | RAIEAQQHLL | RAIEAQQHLL | 629 |
| 5 | TCKSNITGLL | TCKSNITGLL | 630 |
| 9 | KPVVSTQLL | KPVVSTQLL | 631 |
| 10 | KPCVKLTPL | KPCVKLTPLC | 632, 633 |
| 11 | GPKVKQWPL | GPKVKQWPLT | 634, 635 |
| 12 | YPGIKVRQL | YPGIKVRQLC | 636, 637 |

TABLE 2
B37

| Peptide # | Peptide | Seq. Mfg'd & Used | SEQ ID NO: |
|---|---|---|---|
| 2 | TVLDVGDAYF | TVLDVGDAYF | 638 |
| 6 | EPPFLWMGY | EPPFLWMGYE | 639, 640 |
| 7 | VPVKLKPGM | VPVKLKPGMD | 641, 642 |
| 8 | CPKVTFDPI | CPKVTFDPIP | 643, 644 |
| 9 | KPVVSTQLL | KPVVSTQLL | 645 |
| 10 | KPCVKLTPL | KPCVKLTPLC | 646, 647 |
| 11 | GPKVKQWPL | GPKVKQWPLT | 648, 649 |
| 12 | YPGIKVRQL | YPGIKVRQLC | 650, 651 |

TABLE 3
A2

| Peptide # | Peptide | Seq. Mfg'd & Used | SEQ ID NO: |
|---|---|---|---|
| 13 | ILKEPVHGV | ILKEPVHGVY | 652, 653 |
| 14 | QLPEKDSWTV | QLPEKDSWTV | 654 |
| 15 | NLWTVYYGV | NLWTVYYGV | 655 |
| 16 | QMHEDVISL | QMHEDVISLW | 656, 657 |
| 17 | KIEELREHLL | KIEELREHLL | 658 |
| 18 | DMVNQMHEDV | DMVNQMHEDV | 659 |
| 19 | GLKKKKSVTV | GLKKKKSVTV | 660 |
| 20 | ELHPDKWTV | ELHPDKWTVQ | 661 |

TABLE 4
A11

| peptide # | Peptide | Seq. Mfg'd & Used | SEQ ID NO: |
|---|---|---|---|
| 21 | IYQEPFKNLK | IYQEPFKNLK | 662 |
| 22 | VTFDPIPIHY | VTFDPIPIHY | 663 |
| 23 | TVQCTHGIK | TVQCTHGIKP | 664, 665 |
| 24 | NTPIFALKKK | NTPIFALKKK | 666 |
| 25 | LVDFRELNIK | LVDFRELNKR | 667, 668 |
| 26 | PGMDGPKVK | PGMDGPKVKQ | 669, 670 |
| 27 | GIPHPAGLKK | GIPHPAGLKK | 671 |
| 28 | FTTPDKKHQK | FTTPDKKHQK | 672 |

Conclusion. Regionalized CTL epitopes can be incorporated into a range of existin vaccine strategies, e.g. vectored vaccines, DNA vaccines, and recombinant protein vaccines. This approach also permit the development of novel regionalized HIV vaccine and therapeutic interventions. Alternatively, such regional CTL epitopes, collectively covering virtually all regionally-transmitted strains and prevalent HLA types could be combined into a universal HIV vaccine.

### EXAMPLE 3

### A “World Clade” HIV Vaccine

HLA A Variation in Populations. The distribution of MHC proteins varies from population to population. In general, the HLA—foreign peptide interaction is governed by the sequence of the peptide: each allele has a particular and specific pattern, or motif, and the set of foreign peptides able to bind in the binding groove of the HLA allele is determined by the sequence of the foreign peptide. Although the distribution of MHC proteins in populations inhabiting different regions of the world may restrict, to some extent, the relevance of selected epitopes in different human populations, means to surmount this difficulty have been proposed. For example, identification of CTL epitopes that may be recognized in the context of more than one MHC, such as “promiscuous” or “clustered” MHC binding regions, may permit the development of vaccines that effectively protect genetically diverse human populations. For example, if an HIV-1 peptide could be identified that would bind and be presented by MHC alleles −A2, −A1, and −A20 proteins, it is likely that it would be presented in the context of MHC of approximately 25% of Zaireans (Congolese) and greater than 50% of North American Caucasians. We and others have proposed that prospectively identifying and including such “promiscuous” CTL and Th epitopes in novel HIV-1 vaccines may enhance the utility of these vaccines in a wide range of HIV-1 endemic countries. See Haynes, 348 Lancet 933-937 (1996); Cease & Berzofsky, 12 Annu. Rev. Immunol. 923-989 (1994); Bona et al., 126(19) Immunology Today 126-130 (1998); Brander & Walker, in HIV Immunology Database 1995, Korber & Meyers, eds. (Los Alamos National Laboratories, New Mexico, 1996); Berzofsky et al., 88(3) J. Clin. Invest. 876-84 (1991); and Ward et al., in HIV Immunology Database 1995, Korber & Meyers, eds. (Los Alamos National Laboratories, New Mexico, 1996)).

Database of Conserved HIV-1 MHC Ligands. We prospectively identified regions that are conserved across the maximum number of subtypes (“cross-clade”) and possessing an EpiMatrix score indicative of MHC binding potential for a number of MHC molecules representing the most prevalent HLA alleles (“promiscuous”), and has selected, or weighted, the selection of potential CTL epitopes for the final vaccine construct such that HLA alleles prevalent in HIV-endemic regions of the world are adequately represented. These are highly conserved, promiscuous peptides. Eighty peptides have been synthesized, and binding studies have been intitiated for peptides representing the following HLA alleles: A2, A11, B35, and B7. Studies of peptides representing the following alleles: A1, A3, A24, A31, A33, B12 (44), B17, B53, Cw3, and Cw4 are next in order of priority.

Research Lab Tools; EpiMatrix. EpiMatrix is a matrix-based algorithm that ranks 10 amino acid long segments, overlapping by 9 amino acids, from any protein sequence by estimated probability of binding to a selected MHC molecule. The procedure for developing matrix motifs was published by Schafer et al, 16 Vaccine 1998 (1998). We have constructed matrix motifs for 32 HLA class I alleles, one murine allele (H-2 Kd) and several human class II alleles. Putative MHC ligands are selected by scoring each 10-mer frame in a protein sequence. This score, or estimated binding probability (EBP), is derived by comparing the sequence of the 10-mer to the matrix of 10 amino acid sequences known to bind to each MHC allele. Retrospective studies have demonstrated that EpiMatrix accurately predicts published MHC ligands (Jesdale et al., in Vaccines '97 (Cold Spring Harbor Press, Cold Spring Harbor, N.Y., 1997)).

An additional feature of EpiMatrix is that it can measure the MHC binding potential of each 10 amino acid long snapshot to a number of human HLA, and therefore can be used to identify regions of MHC binding potential clustering. Other laboratories have confirmed cross-presentation of peptides within HLA “superfamilies” (A11, A3, A31, A33 and A68) (Jesdale et al., in Vaccines '97 (Cold Spring Harbor Press, Cold Spring Harbor, N.Y., 1997)). Presumably, vaccines containing such “clustered” or promiscuous epitopes will have an advantage over vaccines composed of epitopes that are not “clustered. In work performed in the TB/HIV Research Lab, we have confirmed cross-MHC binding that was predicted by EpiMatrix.

Peptides Selected for Conservation Across Clades and for CTL Response. The staff of the Los Alamos National Laboratory HIV-1 Sequence Database has compiled a list of HIV-1 sequences which are believed to be representative of currently available HIV-1 sequences. Such representative lists are available for each of the HIV genes/proteins (gag, pol, gag, vpu, rev, env, nef, vif, vpr), although the more heavily sequenced genes (particularly env) have considerably longer lists. It is from these lists that well-conserved putative ligands have been defined.

The list for each protein was analyzed independently. We used a computer program called Conservatrix to find conserved regions. Conservatrix divides each sequence from each isolate into ten amino acid-long strings that overlap by nine amino acids. Then Conservatrix compares each of these strings to all of the other strings using a spreadsheet program that orders the strings from those which were in many of the sequences to those which were unique. These ordered lists represent the first step in the analysis. Strings that were present in “more” (>50 for env, >25 for gag, etc.) HIV-1 isolates were selected for the next phase of the analysis. For example, in the case of env, 478 strings were conserved in more than 50 HIV-1 isolates and were analyzed, using EpiMatrix, for MHC binding potential clustering.

The next step was to identify which of the conserved sequences were likely to be MHC ligands (and putatively, CTL epitopes). EpiMatrix yields a “score” for each of the strings it analyzes. The somewhat arbitrary score of 20% estimated binding probability (EBP) was defined as the cut-off for this step in the analysis. This cut-off is probably too high (too specific, not sensitive enough). The complete list of conserved sequences has been archived.

To continue using env as an example, of the 478 conserved env strings, any peptide with an EBP of greater than 20% for any of the HLA for which EpiMatrix predictions were available was defined as being a putative ligand. 206 of the 478 well conserved strings (43%) met this criterion.

The next step was to select strings that were likely to be ligands for more than one MHC type (MHC binding potential clustering). Histograms have been constructed which indicate which regions stimulate the most HLA types (see, TABLE 5 below).

The list of peptides to be tested has been selected from among those regions that might bind to more than 3 different MHC molecules, paying particular attention to selecting regions that bind to HLA representative of world populations and sequences that were representative of global HIV-1 isolates. A method for weighting predictions by the prevalence of HLA alleles in populations has already been developed in the laboratory. We have performed the first two steps of the peptide selection analysis for env, pol, and gag. Twenty-eight of the peptides selected in this manner are shown in TABLE 5 below, with an abbreviated listing of the strains for which they were identified. Binding studies were also performed.

Reviewing the data shown below, it is clear that we have been able to select from a number of different peptides that are conserved in a wide range of HIV-1 clades and strains. The listing of strains for which each peptide is conserved is limited by space for this application; however, it is should be apparent that there is good cross-clade coverage of different HIV-1 clades.

The following TABLE 5 provides a sample list of peptides that are conserved across HIV-1 clades (only env is shown).

| | conserved in | | | number | |
|---|---|---|---|---|---|
| | # of HIV-1 | reference | | predicted | Putative ligands for these |
| protein | strains | strain | Strains for which sequence is conserved (partial listing) | >20% | alleles |
| env | 70 | SF1703 | Z321 [318] 92UG037 8 [317] TZ017 [310] | 3 | A*6801, B*39011, B*5801 |
| | | | L414 [55] CI211 [50] UG273A [321] DJ264A | | |
| | | | [313] DJ263A [31 | | |
| env | 69 | SF2 | LAI [705] HXB2R [700] NL43 [698] BRVA | 3 | A*3302, A*6801, B*39011 |
| | | | [696] 91US005 11 [708] MN [701] QZ4589 | | |
| | | | [703] JFL [695] SIM | | |
| env | 117 | U455 | SF1703 [224] Z321 [219] 92RW020 5 [205] | 3 | B*39011, B*5101, Cw*0102 |
| | | | 92RW009 14 [217] TZ017 [210] D687 [105] | | |
| | | | UG275A [216] U | | |
| env | 106 | U455 | SF1703 [423] 92RW020 5 [400] 92UG037 8 [410] | 3 | B*2705, B*39011, B*5801 |
| | | | UG275A [413] UG273A [417] CI3271 [148] LBV2310 | | |
| | | | [ | | |
| env | 50 | Z321 | D687 [298] K114 [164] L414 [152] P104 | 3 | B*2705, B*39011, B*5801 |
| | | | [145] PZ61 [143] CI211 [145] DJ264A | | |
| | | | [408] DJ263A [416] DJ2 | | |
| env | 95 | SF2 | SF2B13 [440] LAI [450] HXB2R [445] JB02 | 3 | B7, B*39011, B*5801 |
| | | | [169] NY5CG [437] NL43 [443] JRCSF | | |
| | | | [437] JRFL [436] ALA1 | | |
| env | 114 | SF1703 | 92RW020 5 [283] 92UG037 8 [296] PZ61 [26] | 3 | A*0301, A*1101, B*5801 |
| | | | DJ264A [292] DJ263A [296] CI31 [29] CI451 | | |
| | | | [29] CI3301 [ | | |
| env | 106 | US1 | US2 [558] CM237X [515] 91HT652 11 [556] | 3 | B*39011, B*5101, B*5801 |
| | | | 92UG005 [283] 3202A12 [564] 3202A21 [560] | | |
| | | | MANC [565] | | |
| env | 59 | 92UG021 16 | B_H93TH067A [749] YU2 [753] JRFL [757] | 3 | B14, B*39011, B*5801 |
| | | | JRCSF [758] ALA1 [759] FB_93BR019 10 [760] | | |
| | | | NY5CG [760] | | |
| env | 62 | U455 | SF1703 [695] Z321 [690] 92RW020 5 [671] | 3 | B*39011, B*5101, B*5801 |
| | | | 92UG037 8 [683] D687 [572] UG275A [685] | | |
| | | | VI191A [688] DJ | | |
| env | 98 | Z321 | A_GA1LBV23 [276] SF2 [547] SF2B13 [545] LAI | 4 | A*3101, A*3302, A*6801, B*39011 |
| | | | [553] HXB2R [548] JB02 [275] NL43 [546] | | |
| | | | JRCSF [540] J | | |
| env | 74 | U455 | SF1703 [553] 92RW020 5 [529] 92UG031 7 [547] | 4 | A*3101, A*3302, A*6801, B*39011 |
| | | | 92UG037 8 [541] 92RW009 14 [543] P104 [277] | | |
| | | | CI21 | | |
| env | 145 | SF1703 | 92UG031 7 [119] TZ017 [120] D687 [12] UG275A | 3 | A*0201, A*0301, B*39011 |
| | | | [120] UG273A [120] KENYA [120] CAR4054 | | |
| | | | [120] CAR | | |
| env | 202 | U455 | SF1703 [116] Z321 [116] 92RW020 5 [114] | 5 | B7, B35, B*39011, B*5101, B*5801 |
| | | | 92UG031 7 [115] TZ017 [116] D687 [8] UG275A | | |
| | | | [116] UG27 | | |
| env | 128 | U455 | 92UG031 7 [252] 92RW009 14 [251] D687 [139] | 5 | B7, B35, B*39011, B*5101, B*5801 |
| | | | K114 [1] UG06 [4] UG275A [250] VI191A | | |
| | | | [253] DJ264A | | |
| env | 50 | LAI | HXB2R [794] GP160EN [792] NL43 [792] JRCSF | 3 | A*0301, B*5801, Cw*0702 |
| | | | [786] JRFL [785] ALA1 [787] JH32 [805] | | |
| | | | BAL1 [794] YU | | |
| env | 64 | SF2 | SF2B13 [658] LAI [666] HXB2R [661] GP160EN | 3 | B40, B*4403, B*5801 |
| | | | [659] NY5CG [655] NL43 [659] JRCSF [653] | | |
| | | | JRFL [652] A | | |
| env | 92 | SF1703 | Z321 [687] 92RW020 5 [668] 92UG031 7 [686] | 3 | A*3101, A*3302, B*39011 |
| | | | 92UG037 8 [680] D687 [569] UG275A [682] | | |
| | | | UG273A [68 | | |
| env | 54 | SF1703 | CARSAS [285] Z3 [277] I_GM4 [131] 93BR029 2 | 5 | B8, B35, B*5101, B*5801, Cw*0102 |
| | | | [281] F_H93BR029A [282] 92UG046 8 [283] | | |
| | | | 92UG038 1 | | |
| env | 134 | TZ017 | CARSAS [87] CAR4054 [87] AD_K124A2 [86] | 3 | A*0301, A*1101, A*6801 |
| | | | AD_UG266A2 [87] CA_ZAM184 [87] GX_VI525A2 | | |
| | | | [87] EA_CA | | |
| env | 117 | U455 | UG275A [102] DJ264A [101] DJ263A [101] | 4 | A*0201, A*0301, B*39011, B*5801 |
| | | | DJ258A [101] CAR4054 [102] CAR423A [103] | | |
| | | | LAI [103] HXB2 | | |
| env | 117 | U455 | SF1703 [562] Z321 [557] 92UG031 7 [556] | 5 | A*0201, B7, B35, B*39011, B*5801 |
| | | | 92UG037 8 [550] 92RW009 14 [552] CI211 [284] | | |
| | | | UG273A [5 | | |
| env | 54 | LAI | HXB2R [444] JB02 [168] NY5CG [436] NL43 | 3 | B7, B*39011, B*5801 |
| | | | [442] JRCSF [436] JRFL [435] ALA1 [437] | | |
| | | | JH32 [456] BAL1 [ | | |
| env | 94 | Z321 | 92UG037 8 [252] TZ017 [244] UG273A [256] | 5 | B7, B35, B*39011, B*5101, B*5801 |
| | | | CARSAS [257] A_MLY10A [133] LAI [257] HXB2R | | |
| | | | [252] GP1 | | |
| env | 53 | CAR4054 | FB_93BR019 10 [475] BZ126A [466] RJI03 [347] | 3 | B40, B*4006, B*4006 |
| | | | 93BR020 17 [469] 93BR029 2 [466] AR16 [208] | | |
| | | | AR18 [ | | |
| env | 129 | U455 | SF1703 [486] Z321 [481] 92RW020 5 [462] | 3 | B40, B*4006, B*4006 |
| | | | 92UG031 7 [480] 92RW009 14 [476] P104 [210] | | |
| | | | PZ61 [211] | | |
| env | 53 | 92RW009 14 | BF_RJI01 5 [162] CD_DI2ACD [262] CAR4081 [265] | 3 | A*0301, A*3101, B*39011 |
| | | | U_BU91009A [262] RU570 [226] 93TH968 8 [264] E | | |
| env | 55 | DJ264A | DJ263A[264] B_H93TH067A [257] CB6 [141] CB7 | 3 | A*0301, A*3101, B*39011 |
| | | | [165] CB9 [141] US2 [265] 24612 [237] | | |
| | | | 26807 [253] | | |
| env | 66 | 92UG037 8 | 92RW009 14 [410] DA_MAL [415] CA_ZAM184 [397] | 3 | B8, B*39011, Cw*0102 |
| | | | BF_RJI01 5 [306] FB_AR15 [133] HIV1UG3521 | | |
| | | | [406] | | |
| env | 157 | U455 | SF1703 [36] Z321 [36] 92UG0317 [35] | 3 | A*0301, A*1101, A*6801 |
| | | | 92UG037 8 [34] 92RW009 14 [34] TZ017 [36] | | |
| | | | KENYA [36] CARG | | |

For example, the env peptide KLTPLCVTLN, conserved in 145 different strains on the LANL HIV sequence database, was selected from SF1703 (a clade B strain) and was conserved in SF2, SF2B13, 92UG031.7, TZ017, D687, UG275A, UG273A, CAR4054, CAR4023, CAR423A, A_MLY10A, NY5CG, JRCSF, JRFL, JH32, BAL1,YU2, BRVA, and more, representing several different clades. The HLA class I alleles for which the string is predicted to be a good (greater than 20%) ligand were A2, A0301, and B39.

Prior to selecting peptides for synthesis, we have analyzed the peptides for (1) representation of clade A, C, D and E strains, and (2) adequate representation of potential binding to HLA alleles that are prevalent in countries where clades A, C, D, and E are transmitted. Results from assays performed in the lab to date have shown that a very high proportion of the peptides we selected for our studies bound to T2 cells expressing the appropriate MHC in vitro.

TABLE 6
A1 PEPTIDE SEQUENCES

| | | | | | | SEQ ID. |
|---|---|---|---|---|---|---|
| protein | conservation | Sequence | Ref. strain | ref. start | A\hat{ }0101 | NO: |
| env | 107 | SEEPIPIHYC | U455 | 207 | 30.25% | 30 |
| env | 55 | ELDKWASLWN | US1 | 665 | 2.91% | 31 |
| env | 114 | CTRPNNNTRK | SF1703 | 302 | 1.31% | 332 |
| env | 61 | GVAPTKAKRR | Z321 | 495 | 0.89% | 33 |
| env | 126 | SFNCGGEFFY | U455 | 373 | 0.83% | 34 |
| env | 102 | ITLPCRIKQI | 92UG037.8 | 406 | 0.73% | 35 |
| env | 93 | SSNITGLLLT | AD_K124A2 | 448 | 0.70% | 36 |
| gag | 57 | RLRPGGKKKY | BNG | 20 | 11.73% | 37 |
| gag | 51 | AISPRTLNAW | BZ126B | 144 | 2.23% | 38 |
| gag | 32 | AWEKIRLRPG | BZ126B | 15 | 2.16% | 39 |
| gag | 53 | FRDYVDRFYK | TN243 | 293 | 2.03% | 40 |
| pol | 40 | LKEPVHGVYY | IBNG | 465 | 29.32% | 41 |
| pol | 44 | ETVPVKLKPG | IBNG | 161 | 12.68% | 42 |
| pol | 39 | ETPGIRYQYN | IBNG | 293 | 9.40% | 43 |
| pol | 46 | QKEPPFLWMG | U455 | 376 | 8.33% | 44 |
| pol | 39 | NNETPGIRYQ | IBNG | 291 | 3.29% | 45 |
| pol | 46 | TPDKKHQKEP | U455 | 370 | 3.19% | 46 |
| pol | 38 | IPHPAGLKKK | IBNG | 249 | 2.61% | 47 |
| pol | 43 | LVDFRELNKR | U455 | 228 | 2.23% | 48 |
| rev | 13 | SAEPVPLQLP | SF2 | 67 | 22.60% | 49 |
| tat | 7 | RGDPTGPKES | TH475A | 78 | 30.49% | 50 |
| vif | 17 | LADQLIHLYY | IBNG | 102 | 43.60% | 51 |
| vif | 10 | QVDPGLADQL | SF2 | 97 | 8.75% | 52 |
| vpr | 7 | LHSLGQHIYE | D31 | 39 | 0.60% | 53 |
| vpu | 35 | RAEDSGNESE | CM240X | 49 | 1.38% | 54 |

TABLE 7
A2 PEPTIDE SEQUENCES

| | | | | | | SEQ ID. |
|---|---|---|---|---|---|---|
| protein | conservation | sequence | Ref. strain | ref. start | A\hat{ }0201 | NO: |
| env | 91 | NLWVTVYYGV | Z321 | 32 | 82.51% | 55 |
| env | 110 | GIKQLQARVL | U455 | 565 | 72.16% | 56 |
| env | 91 | QLQARVLAVE | U455 | 568 | 63.81% | 57 |
| env | 145 | KLTPLCVTLN | SF1703 | 120 | 50.93% | 58 |
| env | 67 | NMWQEVGKAM | CA16 | 147 | 49.55% | 59 |
| env | 117 | QMHEDIISLW | U455 | 101 | 47.82% | 60 |
| env | 154 | DMRDNWRSEL | CA20 | 193 | 44.72% | 61 |
| gag | 31 | SLYNTVATLY | UG268 | 77 | 76.09% | 62 |
| gag | 25 | ELRSLYNTVA | U455 | 74 | 69.48% | 63 |
| gag | 88 | EMMTACQGVG | U455 | 341 | 63.81% | 64 |
| gag | 58 | DLNTMLNTVG | BZ126B | 181 | 63.81% | 65 |
| pol | 30 | LLWKGEGAVV | U455 | 955 | 99.50% | 66 |
| pol | 40 | ILKEPVHGVY | IBNG | 464 | 96.43% | 67 |
| pol | 27 | KLLWKGEGAV | U455 | 954 | 88.23% | 68 |
| pol | 28 | HLKTAVQMAV | U455 | 885 | 80.90% | 69 |
| pol | 39 | GLKKKKSVTV | U455 | 253 | 74.16% | 70 |
| pol | 48 | ELHPDKWTVQ | U455 | 387 | 70.39% | 71 |
| pol | 31 | KIEELRQHLL | SF2 | 356 | 69.18% | 72 |
| pol | 33 | KLLRGTKALT | SF2 | 436 | 61.17% | 73 |
| rev | 8 | QILVESPTVL | LAI | 101 | 67.94% | 74 |
| tat | 7 | FLNKGLGISY | UG275A | 38 | 10.68% | 75 |
| vif | 10 | DLADQLIHLY | IBNG | 101 | 54.04% | 76 |
| vif | 12 | HIPLGDARLV | IBNG | 56 | 46.44% | 77 |
| vpr | 9 | LLEELKNEAV | LAI | 22 | 87.89% | 78 |
| vpu | 7 | ILAIVVWTIV | U455 | 17 | 89.70% | 79 |

TABLE 8
A3 PEPTIDE SEQUENCES

| | | | | | | SEQ ID |
|---|---|---|---|---|---|---|
| protein | conservation | sequence | Ref. strain | ref. start | | NO: |
| env | 129 | HSFNCGGEFF | U455 | 372 | 60.47% | 80 |
| env | 138 | TLFCASDAKA | U455 | 49 | 58.33% | 81 |
| env | 86 | HSFNCRGEFF | D687 | 259 | 55.44% | 82 |
| env | 174 | SLWDQSLKPC | U455 | 108 | 49.09% | 83 |
| env | 157 | TVYYGVPVWK | U455 | 35 | 48.61% | 84 |
| env | 93 | VSFEPIPIHY | U455 | 206 | 48.61% | 85 |
| env | 114 | CTRPNNNTRK | SF1703 | 302 | 43.25% | 86 |
| gag | 31 | SLYNTVATLY | UG268 | 77 | 49.34% | 87 |
| gag | 31 | LARNCRAPRK | BZ126B | 399 | 32.34% | 88 |
| gag | 57 | RLRPGGKKKY | BNG | 20 | 32.12% | 89 |
| gag | 73 | ILDIRQGPKE | U455 | 278 | 29.11% | 90 |
| pol | 43 | LVDFRELNKLR | U455 | 228 | 52.52% | 91 |
| pol | 27 | QLDCTHLEGK | U455 | 776 | 50.32% | 92 |
| pol | 27 | AVFIHNFKRK | U455 | 893 | 43.98% | 93 |
| pol | 38 | QIIEQLIKKE | SF2 | 675 | 43.01% | 94 |
| pol | 40 | GIPHPAGLKK | IBNG | 248 | 41.81% | 95 |
| pol | 39 | KVYLAWVPAH | SF2 | 685 | 36.86% | 96 |
| pol | 35 | AIFQSSMTKI | SF2 | 313 | 34.57% | 97 |
| pol | 46 | KLVDFRELNK | U455 | 227 | 33.45% | 98 |
| rev | 6 | KILYQSNPYP | UG273A | 20 | 23.70% | 99 |
| tat | 7 | TACNNCYCKK | SF2 | 20 | 62.35% | 100 |
| vif | 6 | ALTALITPKK | MN | 149 | 37.32% | 101 |
| vif | 31 | KLTEDRWNKP | U455 | 168 | 35.02% | 102 |
| vpr | 27 | WTLELLEELK | IBNG | 18 | 22.76% | 103 |
| vpu | 9 | RLIDRIRERA | SC | 42 | 37.32% | 104 |

TABLE 9
A11 PEPTIDE SEQUENCES

| | | | | | | SEQ ID |
|---|---|---|---|---|---|---|
| protein | conservation | sequence | ref. strain | ref. start | | NO: |
| env | 101 | TVQCTHGIKP | U455 | 242 | 52.33% | 105 |
| env | 51 | FAILKCNDKK | BF_RJI01.5 | 121 | 45.11% | 106 |
| env | 134 | NVTENFNMWK | TZ017 | 87 | 38.39% | 107 |
| env | 62 | TITLPCRIKQ | 92UG037.8 | 405 | 38.05% | 108 |
| env | 157 | TVYYGVPVWK | U455 | 35 | 33.47% | 109 |
| env | 114 | CTRPNNNTRK | SF1703 | 302 | 33.05% | 110 |
| env | 135 | VTENFNMWKN | TZ017 | 88 | 32.62% | 111 |
| gag | 57 | IRLRPGGKKK | BNG | 19 | 57.42% | 112 |
| gag | 64 | KIRLRPGGKK | BZ126B | 18 | 47.32% | 113 |
| gag | 91 | LVQNANPDCK | U455 | 318 | 33.37% | 114 |
| gag | 43 | ARNCRAPRKK | BZ126B | 400 | 25.16% | 115 |
| pol | 38 | FTTPDKKHQK | IBNG | 369 | 64.26% | 116 |
| pol | 40 | GIPHPAGLKK | IBNG | 248 | 63.28% | 117 |
| pol | 43 | TTPDKKHQKE | IBNG | 370 | 62.39% | 118 |
| pol | 38 | IPHPAGLKKK | IBNG | 249 | 58.91% | 119 |
| pol | 27 | AVFIHNFKRK | U455 | 893 | 57.99% | 120 |
| pol | 40 | NTPVFAIKKK | U455 | 211 | 57.88% | 121 |
| pol | 45 | PGMDGPKVKQ | IBNG | 169 | 57.65% | 122 |
| pol | 27 | QVRDQAEHLK | IBNG | 879 | 55.58% | 123 |
| rev | 9 | PTVLESGTKE | LAI | 107 | 31.68% | 124 |
| tat | 7 | TACNNCYCKK | SF2 | 20 | 70.97% | 125 |
| vif | 6 | IKPPLPSVKK | MN | 159 | 51.98% | 126 |
| vif | 6 | ALTALITPKK | MN | 149 | 44.77% | 127 |
| vpr | 27 | WTLELLEELK | IBNG | 18 | 21.41% | 128 |
| vpu | 8 | WTIVFIEYRK | CDC42 | 23 | 31.58% | 129 |

TABLE 10
A24 PEPTIDE SEQUENCES

| | | | | | | SEQ ID |
|---|---|---|---|---|---|---|
| protein | conservation | Sequence | ref. strain | ref. start | A\hat{ }2401 | NO: |
| env | 67 | RYLKDQQLLG | SF1703 | 590 | 58.82% | 130 |
| env | 58 | SYHRLRDLLL | DA_MAL | 770 | 0.18% | 131 |
| pol | 38 | IYQEPFKNLK | U455 | 495 | 15.49% | 132 |
| pol | 27 | VYYDPSKDLI | LAI | 484 | 0.01% | 133 |
| vif | 17 | YYFDCFSESA | JRCSF | 110 | 0.02% | 134 |
| vpr | 18 | PYNEWTLELL | SF2 | 14 | 0.01% | 135 |

TABLE 11
A31 PEPTIDE SEQUENCES

| | | | | | A\hat{ }3101 | SEQ ID |
|---|---|---|---|---|---|---|
| protein | conservation | sequence | ref. strain | ref. start | (10-mers) | NO: |
| env | 92 | MIVGGLIGLR | SF1703 | 692 | 71.89% | 136 |
| env | 53 | SLAEEEIIIR | 92RW009.14 | 263 | 71.89% | 137 |
| env | 98 | IVQQQNNLLR | Z321 | 548 | 39.79% | 138 |
| env | 74 | IVQQQSNLLR | U455 | 541 | 39.79% | 139 |
| env | 55 | SLAEEEVVIR | DJ264A | 260 | 39.79% | 140 |
| env | 101 | STVQCTHGIR | SF1703 | 249 | 13.63% | 141 |
| env | 83 | LQARVLAVER | U455 | 569 | 13.63% | 142 |
| gag | 42 | LVWASRELER | BNG | 34 | 85.94% | 143 |
| gag | 37 | IVWASRELER | K98 | 34 | 85.94% | 144 |
| gag | 89 | IILGLNKIVR | U455 | 262 | 71.89% | 145 |
| gag | 44 | QMVHQAISPR | BZ126B | 139 | 71.89% | 146 |
| pol | 27 | KIQNFRVYYR | U455 | 933 | 99.88% | 147 |
| pol | 43 | LVDFRELNKR | U455 | 228 | 39.79% | 148 |
| pol | 46 | KLVDFRELNK | U455 | 227 | 18.66% | 149 |
| pol | 40 | SMTKILEPFR | U455 | 317 | 13.63% | 150 |
| pol | 29 | SINNETPGIR | SF2 | 289 | 13.63% | 151 |
| pol | 26 | GIGGYSAGER | U455 | 904 | 13.63% | 152 |
| pol | 39 | TFYVDGAANR | U455 | 593 | 11.15% | 153 |
| pol | 30 | SQIIEQLIKK | SF2 | 674 | 8.24% | 154 |
| rev | 34 | GTRQARRNRR | SF2 | 33 | 2.65% | 155 |
| tat | 10 | KTACTNCYCK | HXB2R | 19 | 7.36% | 156 |
| vif | 6 | AILGHIVSPR | JRCSF | 123 | 71.89% | 157 |
| vif | 33 | QVMIVWQVDR | U455 | 6 | 59.46% | 158 |
| vpr | 27 | LQQLLFIHFR | U455 | 64 | 39.79% | 159 |
| vpu | 21 | KILRQRKIDR | CM240X | 32 | 97.23% | 160 |

TABLE 12
A33 PEPTIDE SEQUENCES

| | | | | | A*3302 | SEQ ID |
|---|---|---|---|---|---|---|
| protein | conservation | sequence | ref. strain | ref. start | (10-mers) | NO: |
| env | 51 | EITTHSFNCR | UG23 | 93 | 76.02% | 161 |
| env | 98 | IVQQQNNLLR | Z321 | 548 | 23.98% | 162 |
| env | 92 | MIVGGLIGLR | SF1703 | 692 | 23.98% | 163 |
| env | 91 | ASITLTVQAR | U455 | 526 | 23.98% | 164 |
| env | 82 | AIAVAEGTDR | SF2B13 | 816 | 23.98% | 165 |
| env | 74 | IVQQQSNLLR | U455 | 541 | 23.98% | 166 |
| env | 69 | AVLSIVNRVR | SF2 | 699 | 23.98% | 167 |
| gag | 89 | IILGLNKIVR | U455 | 262 | 23.98% | 168 |
| gag | 62 | GVGGPGHKAR | U455 | 348 | 23.98% | 169 |
| gag | 52 | YVDRFYKTLR | ELI | 240 | 23.98% | 170 |
| gag | 48 | YSPVSILDIR | ZAM19 | 157 | 23.98% | 171 |
| pol | 27 | ELKKIIGQVR | U455 | 871 | 52.05% | 172 |
| pol | 43 | LVDFRELNKR | U455 | 228 | 23.98% | 173 |
| pol | 42 | GSDLEIGQHR | U455 | 344 | 23.98% | 174 |
| pol | 40 | SMTKILEPFR | U455 | 317 | 23.98% | 175 |
| pol | 29 | SINNETPGIR | SF2 | 289 | 23.98% | 176 |
| pol | 26 | GIGGYSAGER | U455 | 904 | 23.98% | 177 |
| pol | 45 | EAELELAENR | U455 | 452 | 8.65% | 178 |
| pol | 27 | KIQNERVYYR | U455 | 933 | 1.22% | 179 |
| rev | 32 | EGTRQARRNR | SF2 | 32 | 8.65% | 180 |
| tat | 47 | GISYGRKKRR | DJ263A | 44 | 23.98% | 181 |
| vif | 12 | EVHIPLGDAR | IBNG | 54 | 76.02% | 182 |
| vif | 33 | QVMIVWQVDR | U455 | 6 | 23.98% | 183 |
| vpr | 7 | HSRIGITRQR | JRCSF | 78 | 23.98% | 184 |
| vpu | 6 | DSGNESEGDR | ELI | 52 | 76.02% | 185 |

TABLE 13
A68 PEPTIDE SEQUENCES

| | | | | | A*6801 | SEQ ID |
|---|---|---|---|---|---|---|
| protein | conservation | sequence | ref. strain | ref. start | (10-mers) | NO: |
| env | 61 | GVAPTKAKRR | Z321 | 495 | 65.96% | 186 |
| env | 69 | AVLSIVNRVR | SF2 | 699 | 54.21% | 187 |
| env | 98 | IVQQQNNLLR | Z321 | 548 | 34.15% | 188 |
| env | 74 | IVQQQSNLLR | U455 | 541 | 34.15% | 189 |
| env | 157 | TVYYGVPVWK | U455 | 35 | 21.52% | 190 |
| env | 134 | NVTENFNMWK | TZ017 | 87 | 21.52% | 191 |
| env | 101 | STVQCTHGIR | SF1703 | 249 | 17.62% | 192 |
| gag | 62 | GVGGPGHKAR | U455 | 348 | 54.21% | 193 |
| gag | 26 | GVGGPSHKAR | VI310 | 351 | 54.21% | 194 |
| gag | 42 | LVWASRELER | BNG | 34 | 45.90% | 195 |
| gag | 37 | IVWASRELER | K98 | 34 | 45.90% | 196 |
| pol | 27 | AVFIHNFKRK | U455 | 893 | 39.20% | 197 |
| pol | 43 | LVDFRELNKR | U455 | 228 | 34.15% | 198 |
| pol | 32 | LVEICTEMEK | SF2 | 189 | 31.46% | 199 |
| pol | 27 | QVRDQAEHLK | IBNG | 879 | 31.46% | 200 |
| pol | 42 | LVKLWYQLEK | U455 | 576 | 21.52% | 201 |
| pol | 38 | FTTPDKKHQK | IBNG | 369 | 6.44% | 202 |
| pol | 35 | DSWTVNDIQK | U455 | 404 | 5.56% | 203 |
| pol | 40 | NTPVFAIKKK | U455 | 211 | 3.41% | 204 |
| rev | 34 | GTRQARRNRR | SF2 | 33 | 7.44% | 205 |
| tat | 10 | KTACTNCYCK | HXB2R | 19 | 9.51% | 206 |
| vif | 12 | EVHIPLGDAR | IBNG | 54 | 65.96% | 207 |
| vif | 33 | QVMIVWQVDR | U455 | 6 | 54.21% | 208 |
| vpr | 27 | WTLELLEELK | IBNG | 18 | 15.76% | 209 |
| vpu | 6 | DSGNESEGDR | ELI | 52 | 24.23% | 210 |

TABLE 14
B7 PEPTIDE SEQUENCES

| | | | | | | SEQ ID |
|---|---|---|---|---|---|---|
| protein | conservation | sequence | ref. strain | ref. start | B7 | NO: |
| env | 128 | KPVVSTQLLL | U455 | 250 | 67.23% | 211 |
| env | 94 | RPVVSTQLLL | Z321 | 253 | 62.56% | 212 |
| env | 202 | KPCVKLTPLC | U455 | 115 | 43.65% | 213 |
| env | 54 | RCSSNITGLL | LAI | 449 | 32.95% | 214 |
| env | 84 | APTKAKRRVV | Z321 | 497 | 30.13% | 215 |
| env | 117 | RAIEAQQHLL | U455 | 550 | 28.51% | 216 |
| env | 72 | GPCKNVSTVQ | SF1703 | 243 | 25.30% | 217 |
| gag | 58 | TPQDLNTMLN | UG268 | 175 | 50.10% | 218 |
| gag | 30 | TPQDLNMMLN | AD_K124 | 180 | 49.09% | 219 |
| gag | 60 | GPGHKARVLA | U455 | 351 | 45.50% | 220 |
| gag | 74 | APRKKGCWKC | U455 | 401 | 38.60% | 221 |
| pol | 32 | QPDKSESELV | SF2 | 664 | 55.70% | 222 |
| pol | 43 | GPKVKQWPLT | U455 | 172 | 43.22% | 223 |
| pol | 34 | SPAIFQSSMT | SF2 | 311 | 21.23% | 224 |
| pol | 44 | SPIETVPVKL | U455 | 157 | 18.90% | 225 |
| pol | 31 | KIEELRQHLL | SF2 | 356 | 17.10% | 226 |
| pol | 27 | QVRDQAEHLK | IBNG | 879 | 16.74% | 227 |
| pol | 28 | LVSQIIEQLI | SF2 | 672 | 11.11% | 228 |
| pol | 29 | IPAETGQETA | U455 | 803 | 11.04% | 229 |
| rev | 23 | LPPLERLTLD | SF2 | 75 | 68.27% | 230 |
| tat | 8 | GPKE$KKKVE | TH475A | 83 | 14.25% | 231 |
| vif | 7 | KPPLPSVTKL | LAI | 160 | 43.22% | 232 |
| vif | 10 | KPPLPSVKKL | U455 | 160 | 38.19% | 233 |
| vpr | 11 | FPRIWLHSLG | JRCSF | 34 | 65.66% | 234 |
| vpu | 6 | LVILAIVALV | TZ012 | 4 | 8.00% | 235 |

TABLE 15
B8 PEPTIDE SEQUENCES

| | | | | | | SEQ ID |
|---|---|---|---|---|---|---|
| protein | conservation | sequence | ref. strain | ref. start | B8 | NO: |
| env | 54 | NAKTIIVQLN | SF1703 | 286 | 36.95% | 236 |
| env | 56 | PTKAKRRVVQ | SF2 | 496 | 36.67% | 237 |
| env | 119 | LYKYKVVKIE | U455 | 476 | 32.46% | 238 |
| env | 66 | TLPCRIKQII | 92UG037.8 | 407 | 24.36% | 239 |
| env | 105 | VPVWKIEATTT | SF2 | 41 | 23.42% | 240 |
| env | 131 | VWGIKQLQAR | U455 | 563 | 21.82% | 241 |
| env | 64 | DAKAYDTEVH | 92RW020.5 | 54 | 20.93% | 242 |
| gag | 43 | FNCGKEGHLA | U455 | 387 | 26.43% | 243 |
| gag | 39 | NAWVKVVEEK | BZ126B | 151 | 20.49% | 244 |
| gag | 47 | DCKTILKALG | SF2 | 331 | 19.96% | 245 |
| gag | 49 | NAWVKVIEEK | BNG | 150 | 19.32% | 246 |
| pol | 39 | GLKKKKSVTV | U455 | 253 | 73.44% | 247 |
| pol | 43 | GPKVKQWPLT | U455 | 172 | 72.05% | 248 |
| pol | 46 | AIKKKDSTKW | U455 | 216 | 51.14% | 249 |
| pol | 46 | FAIKKKDSTK | U455 | 215 | 49.32% | 250 |
| pol | 36 | QHRTKIEELR | SF2 | 352 | 43.87% | 251 |
| pol | 27 | ELKKIIGQVR | U455 | 871 | 35.67% | 252 |
| pol | 38 | AGLKKKKSVT | U455 | 252 | 25.94% | 253 |
| pol | 26 | GIKVKQLCKL | U455 | 427 | 25.33% | 254 |
| rev | 7 | IIKILYQSNP | UG273A | 18 | 7.75% | 255 |
| tat | 16 | ESKKKVERET | SF2 | 86 | 65.88% | 256 |
| vif | 9 | TPKKIKPPLP | LAI | 155 | 22.95% | 257 |
| vif | 27 | AGHNKVGSLQ | U455 | 137 | 22.95% | 258 |
| vpr | 22 | EAIIRILQQL | U455 | 58 | 19.22% | 259 |
| vpu | 7 | WLIDRIRERA | TZ023 | 41 | 6.13% | 260 |

TABLE 16
B14 PEPTIDE SEQUENCES

| | | | | | | SEQ ID |
|---|---|---|---|---|---|---|
| protein | conservation | sequence | ref. strain | ref. start B14 | NO: | |
| env | 68 | ERYLKDQQLL | US2 | 582 | 97.12% | 261 |
| env | 59 | FSYHRLRDLL | 92UG021.16 | 749 | 20.43% | 262 |
| env | 106 | EAQQHLLQLT | US1 | 562 | 9.22% | 263 |
| env | 178 | MRDNWRSELY | SF1703 | 480 | 0.35% | 264 |
| env | 50 | CRIKQIVNMW | Z321 | 418 | 0.28% | 265 |
| env | 56 | PTKAKRRVVQ | SF2 | 496 | 0.16% | 266 |
| env | 66 | TLPCRIKQII | 92UG037.8 | 407 | 0.13% | 267 |
| gag | 37 | DRFFKTLRAE | U455 | 294 | 44.20% | 268 |
| gag | 52 | DRFYKTLRAE | TN243 | 298 | 36.29% | 269 |
| gag | 26 | ERFAVNPGLL | SF2 | 42 | 5.50% | 270 |
| gag | 31 | SLYNTVATLY | UG268 | 77 | 0.25% | 271 |
| pol | 32 | GAANRETKLG | U455 | 598 | 0.40% | 272 |
| pol | 31 | NRETKLGKAG | U455 | 601 | 0.08% | 273 |
| pol | 45 | KLVGKLNWAS | U455 | 413 | 0.03% | 274 |
| pol | 30 | EPFRKQNPDI | SF2 | 324 | 0.01% | 275 |
| pol | 33 | LTEEKIKALV | SF2 | 181 | 0.01% | 276 |
| pol | 44 | WTVNDIQKLV | U455 | 406 | 0.01% | 277 |
| rev | 35 | TRQARRNRRR | SF2 | 34 | 4.66% | 278 |
| tat | 35 | GRKKRRQRRR | SF2 | 48 | 2.30% | 279 |
| vif | 27 | DRWNKPQKTK | SF2 | 172 | 53.54% | 280 |
| vif | 22 | ERDWHLGQGV | IFA86 | 76 | 6.68% | 281 |
| vpr | 6 | QREPHNEWTL | LAI | 11 | 1.91% | 282 |
| vpu | 19 | LRQRKIDRLI | LM | 33 | 4.71% | 283 |

TABLE 17
B15 (10-mers) PEPTIDE SEQUENCES

| | | | | | B\hat{ }1501 | SEQ ID |
|---|---|---|---|---|---|---|
| protein | conservation | sequence | ref. strain | ref. start | (10-mers) | NO: |
| env | 93 | DLRSLCLFSY | DJ259A | 735 | 66.56% | 284 |
| env | 101 | QQHLLQLTVW | SF2 | 561 | 0.47% | 285 |
| gag | 57 | RLRPGGKKKY | BNG | 20 | 36.98% | 286 |
| gag | 31 | SLYNTVATLY | UG268 | 77 | 2.43% | 287 |
| gag | 71 | DIRQGPKEPF | U455 | 280 | 0.38% | 288 |
| gag | 83 | RQANFLGKIW | U455 | 423 | 0.13% | 289 |
| pol | 40 | ILKEPVHGVY | IBNG | 464 | 53.38% | 290 |
| pol | 33 | GQGQWTYQIY | SF2 | 488 | 42.73% | 291 |
| pol | 28 | VQMAVFIHNF | U455 | 890 | 42.73% | 292 |
| pol | 44 | IQKLVGKLNW | U455 | 411 | 4.02% | 293 |
| pol | 38 | EQLIKKEKVY | SF2 | 678 | 1.83% | 294 |
| pol | 47 | YQYNVLPQGW | U455 | 298 | 0.13% | 295 |
| pol | 46 | HQKEPPFLWM | U455 | 375 | 0.01% | 296 |
| rev | 11 | LLKTVRLIKF | MN | 12 | 75.68% | 297 |
| tat | 7 | FLNKGLGISY | UG275A | 38 | 17.27% | 298 |
| vif | 10 | DLADQLIHLY | IBNG | 101 | 1.83% | 299 |
| vif | 23 | HLGQGVSIEW | IFA86 | 80 | 0.30% | 300 |
| vpr | 23 | ILQQLLFIHF | U455 | 63 | 28.91% | 301 |

TABLE 18
B27 PEPTIDE SEQUENCES

| | | | | | | SEQ ID |
|---|---|---|---|---|---|---|
| protein | conservation | sequence | ref. strain | ref. start | B\hat{ }2705 | NO: |
| env | 108 | CRIKQIINMW | U455 | 411 | 94.41% | 302 |
| env | 50 | CRIKQIVNMW | Z321 | 418 | 85.77% | 303 |
| env | 82 | RRVVQREKRA | SF1703 | 508 | 16.62% | 304 |
| env | 88 | KRRVVQREKR | SF1703 | 507 | 13.63% | 305 |
| env | 103 | RRVVEREKRA | U455 | 496 | 12.89% | 306 |
| env | 51 | IRSENLTNNA | CI3301 | 5 | 12.89% | 307 |
| env | 90 | KRRVVEREKR | U455 | 495 | 7.04% | 308 |
| gag | 81 | KIRWIILGLNK | BZ126B | 261 | 25.12% | 309 |
| gag | 71 | IRQGPKEPFR | U455 | 281 | 14.39% | 310 |
| gag | 57 | IRLRPGGKKK | BNG | 19 | 12.19% | 311 |
| gag | 43 | ARNCRAPRKK | BZ126B | 400 | 8.94% | 312 |
| pol | 26 | KRKGGIGGYS | U455 | 900 | 33.92% | 313 |
| pol | 38 | KRTQDFWEVQ | U455 | 236 | 5.76% | 314 |
| pol | 30 | HRTKIEELRQ | SF2 | 353 | 0.61% | 315 |
| pol | 27 | KQNPDIVIYQ | SF2 | 328 | 0.37% | 316 |
| pol | 26 | VRDQAEHLKT | IBNG | 880 | 0.30% | 317 |
| pol | 40 | IRYQYNVLPQ | IBNG | 297 | 0.13% | 318 |
| pol | 29 | KALTEVIPLT | SF2 | 442 | 0.11% | 319 |
| pol | 37 | WGFTTPDKKH | IBNG | 367 | 0.09% | 320 |
| rev | 13 | GRSAEPVPLQ | SF2 | 65 | 47.75% | 321 |
| tat | 9 | RRAPQDSQTH | SF2 | 56 | 13.07% | 322 |
| vif | 32 | NRWQVMIVWQ | U455 | 3 | 10.24% | 323 |
| vif | 11 | ARLVITTYWG | LAI | 62 | 8.14% | 324 |
| vpr | 6 | SRIGIIQQRR | SF2 | 79 | 97.28% | 325 |
| vpu | 19 | LRQRKIDRLI | LAI | 33 | 0.63% | 326 |

TABLE 19
B35 PEPTIDE SEQUENCES

| | | | | | | SEQ ID |
|---|---|---|---|---|---|---|
| protein | conservation | sequence | ref. strain | ref. start | B35 | NO: |
| env | 202 | KPCVKLTPLC | U455 | 115 | 94.43% | 327 |
| env | 128 | KPVVSTQLLL | U455 | 250 | 94.43% | 328 |
| env | 94 | RPVVSTQLLL | Z321 | 253 | 94.43% | 329 |
| env | 100 | CPKVSFEPIP | U455 | 203 | 83.30% | 330 |
| env | 117 | RAIEAQQHLL | U455 | 550 | 53.09% | 331 |
| env | 54 | NAKTIIVQLN | SF1703 | 286 | 39.25% | 332 |
| env | 85 | LPCRIKQIIN | SF1703 | 421 | 34.07% | 333 |
| gag | 92 | GPKEPFRDYV | U455 | 284 | 99.99% | 334 |
| gag | 32 | GPAATLEEMM | LBV2310 | 335 | 94.57% | 335 |
| gag | 31 | GPGATLEEMM | U455 | 334 | 94.57% | 336 |
| gag | 58 | TPQDLNTMLN | UG268 | 175 | 94.43% | 337 |
| pol | 43 | GPKVKQWPLT | U455 | 172 | 98.24% | 338 |
| pol | 46 | VPVKLKPGMD | IBNG | 163 | 94.57% | 339 |
| pol | 46 | EPPFLWMGYE | U455 | 378 | 94.57% | 340 |
| pol | 44 | TPPLVKLWYQ | U455 | 573 | 94.57% | 341 |
| pol | 34 | SPAIFQSSMT | SF2 | 311 | 94.57% | 342 |
| pol | 28 | EPIVGAETFY | SF2 | 587 | 76.68% | 343 |
| pol | 27 | NPDIVIYQYM | SF2 | 330 | 54.09% | 344 |
| pol | 45 | KPGMDGPKVK | IBNG | 168 | 53.59% | 345 |
| rev | 23 | LPPLERLTLD | SF2 | 75 | 89.28% | 346 |
| tat | 14 | GPKESKKKVE | SF170 | 83 | 82.99% | 347 |
| vif | 9 | TPKKIKPPLP | LAI | 155 | 98.24% | 348 |
| vif | 12 | KSLVKHHMYI | SF2 | 22 | 76.68% | 349 |
| vpr | 11 | FPRIWLHSLG | JRCSF | 34 | 98.24% | 350 |
| vpu | 6 | QPLVILAIVA | TZ023 | 2 | 9.91% | 351 |

TABLE 20
B38 PEPTIDE SEQUENCES

| | | | | | | SEQ ID |
|---|---|---|---|---|---|---|
| protein | conservation | sequence | ref. strain | ref. start | B38 | NO: |
| env | 121 | IHYCAPAGFA | U455 | 213 | 55.70% | 352 |
| env | 115 | MHEDIISLWD | U455 | 102 | 46.23% | 353 |
| env | 59 | YHRLRDLLLI | LAI | 773 | 23.31% 354 | |
| env | 101 | QHLLQLTVWG | SF2 | 562 | 9.57% | 355 |
| env | 119 | THGIKPVVST | U455 | 246 | 9.29% | 356 |
| env | 97 | THGIRPVVST | Z321 | 249 | 9.19% | 357 |
| env | 129 | VHNVWATHAC | U455 | 63 | 9.01% | 358 |
| gag | 95 | GHQAAMQMLK | U455 | 189 | 57.48% | 359 |
| gag | 35 | SHKGRPGNFL | SM145 | 436 | 38.92% | 360 |
| gag | 28 | LHPVHAGPIA | BZ167 | 216 | 23.66% | 361 |
| gag | 45 | VHQAISPRTL | SM145 | 140 | 12.44% | 362 |
| pol | 34 | AHTNDVKQLT | U455 | 514 | 50.97% | 363 |
| pol | 46 | KHQKEPPFLW | U455 | 374 | 47.58% | 364 |
| pol | 36 | QHRTKIEELR | SF2 | 352 | 25.26% | 365 |
| pol | 28 | EHLKTAVQMA | U455 | 884 | 19.21% | 366 |
| pol | 31 | KIEELRQHLL | SF2 | 356 | 14.26% | 367 |
| pol | 32 | QPDKSESELV | SF2 | 664 | 13.64% | 368 |
| pol | 35 | LTEEAELELA | U455 | 449 | 13.51% | 369 |
| pol | 33 | LTEEKIKALV | SF2 | 181 | 10.36% | 370 |
| rev | 13 | SAEPVPLQLP | SF2 | 67 | 13.03% | 371 |
| tat | 21 | KHPGSQPKTA | TH475A | 12 | 22.79% | 372 |
| vif | 18 | IHLYYFDCFS | LAI | 107 | 48.94% | 373 |
| vif | 8 | IHLHYFDCFS | U455 | 107 | 48.94% | 374 |
| vpr | 6 | PHNEWTLELL | LAI | 14 | 17.41% | 375 |
| vpu | 19 | ESEGDQEELS | SF2 | 56 | 10.36% | 376 |

TABLE 21
B39 PEPTIDE SEQUENCES

| | | | | | | SEQ ID |
|---|---|---|---|---|---|---|
| protein | conservation | sequence | ref. strain | ref. start | B*39011 | NO: |
| env | 115 | MHEDIISLWD | U455 | 102 | 58.82% | 377 |
| env | 178 | MRDNWRSELY | SF1703 | 480 | 56.02% | 378 |
| env | 108 | CRIKQIINMW | U455 | 411 | 49.57% | 379 |
| env | 93 | IRPVVSTQLL | Z321 | 252 | 49.57% | 380 |
| env | 50 | CRIKQIVNMW | Z321 | 418 | 49.57% | 381 |
| env | 68 | ERYLKDQQLL | US2 | 582 | 49.57% | 382 |
| env | 59 | YHRLRDLLLI | LAI | 773 | 48.00% | 383 |
| gag | 95 | GHQAAMQMLK | U455 | 189 | 80.51% | 384 |
| gag | 28 | LHPVHAGPIA | BZ167 | 216 | 60.35% | 385 |
| gag | 26 | ERFAVNPGLL | SF2 | 42 | 60.35% | 386 |
| gag | 38 | SRELERFALN | SM145 | 38 | 56.02% | 387 |
| pol | 34 | AHTNDVKQLT | U455 | 514 | 80.51% | 388 |
| pol | 46 | KHQKEPPFLW | U455 | 374 | 75.73% | 389 |
| pol | 28 | EHLKTAVQMA | U455 | 884 | 70.38% | 390 |
| pol | 36 | QHRTKIEELR | SF2 | 352 | 64.99% | 391 |
| pol | 33 | LTEEKIKALV | SF2 | 181 | 58.82% | 392 |
| pol | 27 | VYYDPSKDLI | LAI | 484 | 45.95% | 393 |
| pol | 44 | WTVNDIQKLV | U455 | 406 | 41.59% | 394 |
| pol | 43 | GGNEQVDKLV | U455 | 697 | 41.59% | 395 |
| rev | 13 | GRSAEPVPLQ | SF2 | 65 | 49.57% | 396 |
| tat | 6 | ERETETDPVH | BAL1 | 92 | 49.57% | 397 |
| vif | 23 | WHLGQGVSIE | IFA86 | 79 | 70.38% | 398 |
| vif | 9 | THPRISSEVH | MN | 47 | 60.35% | 399 |
| vpr | 27 | WTLELLEELK | IBNG | 18 | 52.41% | 400 |
| vpu | 19 | LRQRKIDRLI | LAI | 33 | 56.02% | 401 |

TABLE 22
B40 PEPTIDE SEQUENCES

| | | | | | | SEQ ID |
|---|---|---|---|---|---|---|
| protein | conservation | sequence | ref. strain | ref. start | B40 | NO: |
| env | 85 | QEVGKAMYAP | SF2 | 425 | 60.96% | 402 |
| env | 69 | VELLGRRGWE | LAI | 787 | 48.24% | 403 |
| env | 64 | LELDKWASLW | SF2 | 660 | 48.24% | 404 |
| env | 51 | GEFFYCNTSG | U455 | 378 | 44.21% | 405 |
| env | 100 | TEVHNVWATH | 92UG037.8 | 60 | 32.15% | 406 |
| env | 129 | SELYKYKVVK | U455 | 474 | 21.60% | 407 |
| env | 101 | KEATTTLFCA | SF2 | 45 | 21.60% | 408 |
| gag | 29 | IEVKDTKEAL | BZ126B | 92 | 60.96% | 409 |
| gag | 58 | EEAAEWDRLH | U455 | 203 | 48.24% | 410 |
| gag | 51 | GEIYKRWIIL | BZ126B | 257 | 44.21% | 411 |
| gag | 95 | REPRGSDIAG | U455 | 225 | 35.87% | 412 |
| pol | 43 | WEFVNTPPLV | U455 | 568 | 60.96% | 413 |
| pol | 44 | AETFYVDGAA | U455 | 591 | 48.24% | 414 |
| pol | 27 | TELQAIHLAL | SF2 | 632 | 48.24% | 415 |
| pol | 35 | LEVNIVTDSQ | SF2 | 646 | 32.15% | 416 |
| pol | 48 | YELHPDKWTV | U455 | 386 | 27.53% | 417 |
| pol | 38 | NDVKQLTEAV | SF2 | 518 | 24.83% | 418 |
| pol | 36 | TEEAELELAE | U455 | 450 | 24.83% | 419 |
| pol | 40 | GDAYFSVPLD | U455 | 266 | 24.68% | 420 |
| rev | 11 | EELLKTVRLI | MN | 10 | 48.24% | 421 |
| tat | 31 | LEPWKHPGSQ | U455 | 8 | 13.49% | 422 |
| vif | 15 | IEWRKKRYST | LAI | 87 | 21.60% | 423 |
| vif | 8 | IEWRKRRYST | HAN | 88 | 21.60% | 424 |
| vpr | 19 | YETYGDTWAG | SF2 | 47 | 35.87% | 425 |
| vpu | 17 | VEMGHHAPWD | LAI | 68 | 48.24% | 426 |

TABLE 23
B40012 PEPTIDE SEQUENCE

| | | | | | |
|---|---|---|---|---|---|
| | | | | | SEQ ID |
| protein conservation | sequence | ref. strain | ref. start | B*40012 | NO: |

| | | | | | | |
|---|---|---|---|---|---|---|
| rev | 11 | EELLKTVRLI | MN | 10 | 71.53% | 427 |

TABLE 24
B4006 (8 mers) PEPTIDE SEQUENCES

| | | | | | B*4006 | SEQ ID |
|---|---|---|---|---|---|---|
| protein | conservation | sequence | ref. strain | ref. start | (8-mers) | NO: |
| env | 53 | SELYKYKVVE | CAR4054 | 476 | 65.30% | 428 |
| env | 129 | SELYKYKVVK | U455 | 474 | 65.30% | 429 |
| env | 100 | TEVHNVWATH | 92UG037.8 | 60 | 23.25% | 430 |
| env | 51 | GEFFYCNTSG | U455 | 378 | 8.34% | 431 |
| env | 106 | IEAQQHLLQL | SF2 | 558 | 8.00% | 432 |
| env | 73 | REKRAVGIGA | SF1703 | 513 | 5.40% | 433 |
| env | 96 | VEQMHEDIIS | UG275A | 100 | 5.16% | 434 |
| gag | 28 | RELERFAVNP | SF2 | 39 | 66.12% | 435 |
| gag | 93 | KEPFRDYVDR | U455 | 286 | 61.06% | 436 |
| gag | 27 | AEQASQEVKN | IC144 | 303 | 56.69% | 437 |
| gag | 25 | AEQATQEVKN | BZ126B | 304 | 56.69% | 438 |
| pol | 28 | GEAMHGQVDC | U455 | 761 | 66.12% | 439 |
| pol | 41 | RELLKEPVHG | IBNG | 462 | 66.12% | 440 |
| pol | 32 | NEQVDKLVSA | SF2 | 700 | 56.69% | 441 |
| pol | 28 | AEHLKTAVQM | U455 | 883 | 56.69% | 442 |
| pol | 33 | EEKIKALVEI | SF2 | 183 | 56.69% | 44Y |
| pol | 35 | PEKDSWTVNP | U455 | 401 | 48.66% | 444 |
| pol | 29 | IEAEVIPAET | U455 | 798 | 30.65% | 445 |
| pol | 36 | RETKLGKAGY | U455 | 602 | 23.95% | 446 |
| rev | 9 | DEELLKTVRL | MN | 9 | 56.69% | 447 |
| tat | 18 | MEPVDPRLEP | TH475A | 1 | 5.16% | 448 |
| vif | 11 | SESAIRNAIL | JRCSF | 116 | 16.97% | 449 |
| vif | 32 | MENRWQVMIV | U455 | 1 | 5.16% | 450 |
| vpr | 13 | EELKSEAVRH | NL43 | 24 | 65.30% | 451 |
| vpu | 13 | QEELSALVEM | SF2 | 61 | 56.69% | 452 |

TABLE 25
B4006 (9 mers) PEPTIDE SEQUENCES

| | | | | | B*4006 | SEQ ID |
|---|---|---|---|---|---|---|
| protein | conservation | sequence | ref. strain | ref. start | (9-mers) | NO: |
| env | 53 | SELYKYKVVE | CAR4054 | 476 | 55.16% | 453 |
| env | 129 | SELYKYKVVK | U455 | 474 | 55.16% | 454 |
| env | 85 | QEVGKAMYAP | SF2 | 425 | 27.31% | 455 |
| env | 64 | LELDKWASLW | SF2 | 660 | 5.69% | 456 |
| env | 117 | FEPIPIHYCA | A_MLY10A | 91 | 1.03% | 457 |
| env | 101 | KEATTTLFCA | SF2 | 45 | 1.03% | 458 |
| env | 100 | TEVHNVWATH | 92UG037.8 | 60 | 1.03% | 459 |
| gag | 48 | AEWDRLHPVH | U455 | 206 | 55.16% | 460 |
| gag | 79 | EEKAFSPEVI | BZ126B | 158 | 27.31% | 461 |
| gag | 76 | TETLLVQNAN | ZAM18 | 261 | 27.31% | 462 |
| gag | 43 | KETTINEEAAE | TN243 | 202 | 27.31% | 463 |
| pol | 27 | TELQAIHLAL | SF2 | 632 | 55.16% | 464 |
| pol | 44 | AETFYVDGAA | U455 | 591 | 27.31% | 465 |
| pol | 33 | TEEKIKALVE | SF2 | 182 | 27.31% | 466 |
| pol | 39 | KEKVYLAWVP | SF2 | 683 | 27.31% | 467 |
| pol | 43 | WEFVNTPPLV | U455 | 568 | 12.60% | 468 |
| pol | 36 | TEEAELELAE | U455 | 450 | 9.06% | 469 |
| pol | 38 | TEMEKEGKIS | IBNG | 194 | 5.69% | 470 |
| pol | 44 | LELAENREIL | U455 | 455 | 5.69% | 471 |
| rev | 11 | EELLKTVRLI | MN | 10 | 5.69% | 472 |
| vif | 22 | RDWHLGQGVS | IFA86 | 77 | 2.42% | 473 |
| vif | 32 | MENRWQVMIV | U455 | 1 | 1.03% | 474 |
| vpr | 19 | YETYGDTWAG | SF2 | 47 | 27.31% | 475 |
| vpu | 18 | EELSALVEMG | SF2 | 62 | 5.69% | 476 |

TABLE 26
B44 PEPTIDE SEQUENCES

| | | | | | | SEQ ID |
|---|---|---|---|---|---|---|
| protein | conservation | sequence | ref. strain | ref. start | B*4403 | NO: |
| env | 64 | LELDKWASLW | SF2 | 660 | 22.60% | 477 |
| env | 67 | LEITTHSFNC | SF1703 | 373 | 15.03% | 478 |
| env | 229 | DNWRSELYKY | CA20 | 196 | 11.08% | 479 |
| env | 101 | KEATTTLFCA | SF2 | 45 | 10.03% | 480 |
| env | 68 | GDLEITTHSF | SF1703 | 371 | 8.52% | 481 |
| env | 106 | IEAQQHLLQL | SF2 | 558 | 6.99% | 482 |
| env | 82 | QARVLAVERY | U455 | 570 | 5.31% | 483 |
| gag | 51 | GEIYKRWIIL | BZ126B | 257 | 15.03% | 484 |
| gag | 94 | LGLNKIVRMY | U455 | 264 | 13.83% | 485 |
| gag | 26 | EEQNKSKKKA | SF2 | 106 | 7.87% | 486 |
| gag | 49 | QEVKNWMTET | BNG | 308 | 6.99% | 487 |
| pol | 46 | KEPPFLWMGY | U455 | 377 | 48.34% | 488 |
| pol | 39 | NETPGIRYQY | IBNG | 292 | 48.34% | 489 |
| pol | 29 | AETGQETAYF | U455 | 805 | 43.01% | 490 |
| pol | 43 | RELNKRTQDF | U455 | 232 | 43.01% | 491 |
| pol | 36 | RETKLGKAGY | U455 | 602 | 35.46% | 492 |
| pol | 35 | LEIGQHRTKI | SF2 | 348 | 26.06% | 493 |
| pol | 28 | EPIVGAETFY | SF2 | 587 | 12.02% | 494 |
| pol | 38 | TEMEKEGKIS | IBNG | 194 | 10.03% | 495 |
| rev | 11 | EELLKTVRLI | MN | 10 | 17.14% | 496 |
| tat | 10 | QPKTACTNCY | HXB2R | 17 | 4.01% | 497 |
| vif | 9 | GDARLVITTY | LAI | 60 | 19.96% | 498 |
| vif | 7 | GDAKLVITTY | SF2 | 60 | 19.96% | 499 |
| vpr | 20 | EDQGPQREPY | U455 | 6 | 12.02% | 500 |
| vpu | 15 | IAIVVWTIVF | CDC42 | 18 | 6.61% | 501 |

TABLE 27
B51 PEPTIDE SEQUENCES

| | | | | | | SEQ ID |
|---|---|---|---|---|---|---|
| protein | conservation | sequence | ref. strain | ref. start | B*5101 | NO: |
| env | 85 | LPCRIKQIIN | SF1703 | 421 | 90.57% | 502 |
| env | 100 | CPKVSFEPIP | U455 | 203 | 86.77% | 503 |
| env | 53 | VAEGTDRVIE | SF2B13 | 819 | 78.20% | 504 |
| env | 84 | APTKAKRRVV | Z321 | 497 | 74.67% | 505 |
| env | 58 | APTRAKRRVV | U455 | 490 | 72.16% | 506 |
| env | 72 | GPCKNVSTVQ | SF1703 | 243 | 69.54% | 507 |
| env | 56 | GPCTNVSTVQ | KENYA | 235 | 66.81% | 508 |
| gag | 54 | NIPPIPVGEIY | BZ126B | 251 | 83.21% | 509 |
| gag | 26 | NPPIPVGDIY | U455 | 249 | 83.21% | 510 |
| gag | 63 | NANPDCKTIL | VI415 | 325 | 69.27% | 511 |
| gag | 96 | SPRTLNAWVK | UG268 | 143 | 66.81% | 512 |
| pol | 27 | FPISPIETVP | U455 | 154 | 78.42% | 513 |
| pol | 35 | LPEKDSWTVN | U455 | 400 | 76.12% | 514 |
| pol | 29 | WASQIYAGIK | U455 | 420 | 66.53% | 515 |
| pol | 27 | TAVQMAVFIH | U455 | 888 | 63.70% | 516 |
| pol | 43 | QGWKGSPAIF | IBNG | 306 | 63.12% | 517 |
| pol | 28 | SGYIEAEVIP | U455 | 795 | 63.12% | 518 |
| pol | 32 | QPDKSESELV | SF2 | 664 | 49.02% | 519 |
| pol | 43 | GPKVKQWPLT | U455 | 172 | 49.02% | 520 |
| rev | 23 | LPPLERLTLD | SF2 | 75 | 53.90% | 521 |
| tat | 14 | GPKLESKKKVE | SF170 | 83 | 74.67% | 522 |
| vif | 14 | DPDLADQLIH | IBNG | 99 | 94.14% | 523 |
| vif | 10 | DPGLADQLIH | SF2 | 99 | 94.14% | 524 |
| vpr | 20 | EAVRHFPRIW | LAI | 29 | 81.01% | 525 |
| vpu | 6 | QPLVILAIVA | TZ023 | 2 | 72.16% | 526 |

TABLE 28
B51 (9 mers) PEPTIDE SEQUENCES

| | | | | | B*5102 | SEQ ID |
|---|---|---|---|---|---|---|
| protein | conservation | sequence | ref. strain | ref. start | (9-mers) | NO: |
| env | 84 | APTKAKRRVV | Z321 | 497 | 17.61% | 527 |
| env | 58 | APTRAKRRVV | U455 | 490 | 17.61% | 528 |
| env | 85 | LPCRIKQIIN | SF1703 | 421 | 17.61% | 529 |
| env | 128 | KPVVSTQLLL | U455 | 250 | 11.65% | 530 |
| env | 94 | RPVVSTQLLL | Z321 | 253 | 11.65% | 531 |
| env | 72 | GPCKNVSTVQ | SF1703 | 243 | 7.17% | 532 |
| env | 56 | GPCTNVSTVQ | KENYA | 235 | 7.17% | 533 |
| gag | 54 | NPPIPVGEIY | BZ126B | 251 | 13.33% | 534 |
| gag | 26 | NPPIPVGDIY | U455 | 249 | 13.33% | 535 |
| gag | 63 | NANPDCKTIL | VI415 | 325 | 5.91% | 536 |
| gag | 28 | NANPDCKSIL | U455 | 321 | 4.92% | 537 |
| pol | 27 | FPISPIETVP | U455 | 154 | 56.10% | 538 |
| pol | 27 | TAVQMAVFIH | U455 | 888 | 25.48% | 539 |
| pol | 43 | QGWKGSPAIF | IBNG | 306 | 17.61% | 540 |
| pol | 28 | SGYIEAEVIP | U455 | 795 | 15.37% | 541 |
| pol | 45 | KPGMDGPKVK | IBNG | 168 | 13.33% | 542 |
| pol | 26 | GGIGGFIKVR | U455 | 103 | 8.21% | 543 |
| pol | 29 | WASQIYAGIK | U455 | 420 | 4.92% | 544 |
| pol | 45 | KGIGGNEQVD | U455 | 694 | 3.33% | 545 |
| rev | 23 | LPPLERLTLD | SF2 | 75 | 1.44% | 546 |
| tat | 14 | GPKESKKKVE | SF170 | 83 | 6.01% | 547 |
| vif | 9 | IPLGDARLVI | LAI | 57 | 28.77% | 548 |
| vif | 8 | IPLGDAKLVI | SF2 | 57 | 28.77% | 549 |
| vpr | 20 | EAVRHFPRIW | LAI | 29 | 48.56% | 550 |
| vpu | 6 | QPLVILAIVA | TZ023 | 2 | 22.94% | 551 |

TABLE 29
B58 (10 mers) PEPTIDE SEQUENCES

| | | | | | B*5801 | SEQ ID |
|---|---|---|---|---|---|---|
| protein | conservation | sequence | ref. strain | ref. start | (10-mers) | NO: |
| env | 189 | VTVYYGVPVW | U455 | 34 | 72.75% | 552 |
| env | 109 | ITQACPKVSF | U455 | 199 | 68.83% | 553 |
| env | 129 | HSFNCGGEFF | U455 | 372 | 65.14% | 554 |
| env | 86 | HSFNCRGEFF | D687 | 259 | 65.14% | 555 |
| env | 93 | VSFEPIPIHY | U455 | 206 | 53.52% | 556 |
| env | 102 | ITLPCRIKQI | 921JG037.8 | 406 | 48.46% | 557 |
| env | 51 | CSGKLICTTA | SF2 | 597 | 47.67% | 558 |
| gag | 53 | TSTLQEQIGW | K31 | 184 | 71.24% | 559 |
| gag | 42 | ETINEEAAEW | TN243 | 203 | 60.34% | 560 |
| gag | 40 | DTINEEAAEW | U455 | 199 | 60.34% | 561 |
| gag | 36 | PSHKGRPGNF | BZ126B | 437 | 50.55% | 562 |
| pol | 26 | VSAGIRKVLF | SF2 | 707 | 68.83% | 563 |
| pol | 41 | WTYQIYQEPF | U455 | 491 | 68.83% | 564 |
| pol | 45 | STKWRKLVDF | U455 | 222 | 66.78% | 565 |
| pol | 35 | SSMTKILEPF | U455 | 316 | 66.78% | 566 |
| pol | 47 | QATWIPEWEF | U455 | 561 | 62.44% | 567 |
| pol | 45 | NTPPLVKILWY | U455 | 572 | 58.51% | 568 |
| pol | 48 | MGYELHPDKW | U455 | 384 | 54.50% | 569 |
| pol | 40 | ISKIGPENPY | U455 | 201 | 51.73% | 570 |
| rev | 35 | QARRNRRRRW | SF2 | 36 | 65.96% | 571 |
| tat | 9 | FTKKGLGISY | OYI | 38 | 53.52% | 572 |
| vif | 9 | DARLVITTYW | LAI | 61 | 57.54% | 573 |
| vif | 7 | DAKILVITTYW | SF2 | 61 | 57.54% | 574 |
| vpr | 20 | EAVRHFPRIW | LAI | 29 | 53.52% | 575 |
| vpu | 10 | VAAIIAIVVW | SC | 14 | 70.30% | 576 |

TABLE 30
Cw1 PEPTIDE SEQUENCES

| | | | | | | SEQ ID |
|---|---|---|---|---|---|---|
| protein | conservation | Sequence | ref. strain | ref. start | Cw*0102 | NO: |
| env | 54 | NAKTIIVQLN | SF1703 | 286 | 42.05% | 577 |
| env | 66 | TLPCRIKQII | 92UG037.8 | 407 | 42.05% | 578 |
| env | 117 | CAPAGFAILK | U455 | 216 | 19.96% | 579 |
| env | 91 | QLQARVLAVE | U455 | 568 | 19.96% | 580 |
| env | 152 | LTVWGIKQLQ | U455 | 561 | 12.22% | 581 |
| env | 106 | EAQQHLLQLT | US1 | 562 | 12.22% | 582 |
| env | 142 | QLLSGIVQQQ | U455 | 536 | 12.22% | 583 |
| gag | 36 | IWPSHKGRPG | BZ126B | 435 | 42.05% | 584 |
| gag | 66 | RAPRKKGCWK | U455 | 400 | 12.22% | 585 |
| gag | 50 | TLQEQIGWMT | K31 | 186 | 12.22% | 586 |
| gag | 45 | FLQSRPEPTA | SF2 | 450 | 12.22% | 587 |
| pol | 29 | KALTEVIPLT | SF2 | 442 | 42.05% | 588 |
| pol | 28 | NLKTGKYARM | SF2 | 503 | 12.22% | 589 |
| pol | 32 | GAANRETKLG | U455 | 598 | 12.22% | 590 |
| pol | 47 | WVPAHKGIGG | U455 | 689 | 12.22% | 591 |
| pol | 32 | LEPFRKQNPD | SF2 | 323 | 12.22% | 592 |
| pol | 39 | KEPVHGVYYD | IBNG | 466 | 6.87% | 593 |
| pol | 44 | ELAENREILK | U455 | 456 | 6.87% | 594 |
| pol | 43 | GGNEQVDKLV | U455 | 697 | 6.87% | 595 |
| rev | 9 | ILVESPTVLE | LAI | 102 | 6.87% | 596 |
| tat | 6 | DSQTHQASLS | SF2 | 61 | 12.22% | 597 |
| vif | 11 | PLPSVKKLTE | U455 | 162 | 42.05% | 598 |
| vif | 25 | HTGERDWHLG | IBNG | 73 | 6.87% | 599 |
| vpr | 25 | QAPEDQGPQR | U455 | 3 | 6.87% | 600 |
| vpu | 19 | ILRQRKIDRL | CM240X | 33 | 6.87% | 601 |

TABLE 31
Cw7 PEPTIDE SEQUENCES

| | | | | | | SEQ ID |
|---|---|---|---|---|---|---|
| protein | conservation | sequence | ref. strain | ref. start | Cw*0702 | NO: |
| env | 50 | KYWWNLLQYW | LAI | 799 | 71.91% | 602 |
| env | 83 | LRSLCLFSYH | SF1703 | 765 | 68.10% | 603 |
| env | 81 | ARVLAVERYL | U455 | 571 | 59.94% | 604 |
| env | 58 | SYHRLRDLLL | DA_MAL | 770 | 5.24% | 605 |
| env | 146 | FNCGGEFFYC | P104 | 105 | 4.95% | 606 |
| env | 93 | IRPVVSTQLL | Z321 | 252 | 3.38% | 607 |
| env | 58 | IRQGLERALL | U455 | 847 | 3.18% | 608 |
| gag | 32 | LRPGGKKKYR | BNG | 21 | 99.90% | 609 |
| gag | 31 | LYNTVATLYC | K7 | 78 | 94.28% | 610 |
| gag | 74 | FSPEVIPMFS | U455 | 160 | 16.37% | 611 |
| gag | 71 | IRQGPKEPFR | U455 | 281 | 9.78% | 612 |
| pol | 44 | TPPLVKLWYQ | U455 | 573 | 74.16% | 613 |
| pol | 26 | KRKGGIGGYS | U455 | 900 | 70.51% | 614 |
| pol | 46 | IYQYMDDLYV | U455 | 334 | 46.95% | 615 |
| pol | 46 | EPPFLWMGYE | U455 | 378 | 37.86% | 616 |
| pol | 46 | TVLDVGDAYF | U455 | 261 | 27.09% | 617 |
| pol | 42 | QYALGIIQAQ | U455 | 654 | 25.31% | 618 |
| pol | 40 | LKEPVHGVYY | IBNG | 465 | 19.97% | 619 |
| pol | 34 | KQGQGQWTYQ | SF2 | 486 | 17.05% | 620 |
| rev | 22 | LQLPPLERLT | SF2 | 73 | 2.99% | 621 |
| tat | 7 | LNKGLGISYG | UG275A | 39 | 24.44% | 622 |
| vif | 6 | QYLALAALIK | NL43 | 146 | 17.40% | 623 |
| vif | 6 | QYLALAALIT | SF2 | 146 | 17.40% | 624 |
| vpr | 10 | LHGLGQHIYE | IBNG | 39 | 21.14% | 625 |
| Vpu | 11 | VWTIVFIEYR | CDC42 | 22 | 1.78% | 626 |

The HLA A2, A11, A3 and B7 peptides in Tables 7-9 and 14 were tested in vitro, in T2 binding assays and in ELIspot assays.

In vitro evaluation of MHC binding was performed by measuring the ability of exogenously added peptides to stabilize the class I MHC/beta 2 microglobulin structure on the surface of TAP-deficient T2 cell lines. Ljunggren et al., Nature 346:476-80 (1990). Binding assays were not performed for the HLA3 peptides. In vitro evaluation of MHC stabilization by the candidate peptide was performed as previously described herein and following the methods described in Ljunggren, supra, Nijman et al., Eur J Immunol 23:1215-19 (1993) and Brander et al., Clin Exp Immunol 101:107-13 (1995). Fluorescence of viable T2 cells (a marker of peptide binding) was measured as described in Example 1.

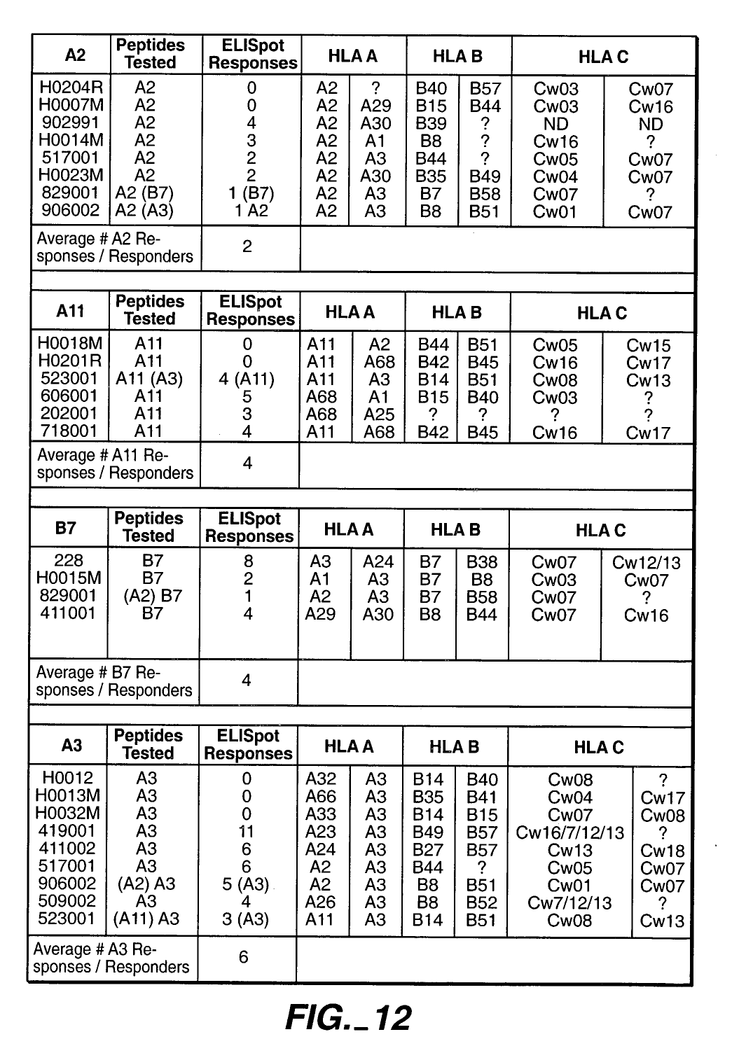

ELIspot assays were performed as follows. Twenty three HIV-1 infected subjects with viral loads below 10,000 copies per ml and absolute CD4 T cell counts above 200 cells per C1 and HIV-1 seronegative control subjects were evaluated in 34 ELIspot assays. In four cases, subjects' PBMC were tested for responses to peptides restricted by more than one HLA allele. See FIG. 12. HLA typing was performed using DNAzol (Gibco/Life Technologies) and HLA SSP ABC Typing Kits (One Lambda, Inc). In some cases, the HLA could not be resolved and these cases are designated wither with multiple alleles (for example, 14/8), where differentiation could not be determined with certainty or with “?”, where no identifiable HLA type could be discerned. FIG. 12. Peripheral blood mononuclear cells (PBMC) were separated from heparinized peripheral blood samples using Lymphoprep (Nycomed Pharma) density centrifugation. The PBMC were pre-incubated with peptide (peptide stimulation) or with PHA (PHA stimulation) or with both (Peptide/PHA stimulation) for 5 to 10 days according to published protocols. In all cases, 20 U/ml IL2 (Sigma) were added 2 or 3 days after cultures were initiated and every 2 days thereafter. PVMCs were harvested after stimulation and plated at 10,000 to 100,000 cells per well in an ELIspot plate (Millipore, Inc.) that was precoated with Mouse anti-human IFN gamma monoclonal antibody (Pharmingen), 15 μg/ml. All ELIspot assays were performed using a single peptide per well. At the time of the final assay, target peptides were added at 10 μg/ml concentration to wells and incubated for 18-20 hours. Autologous PBMC or T2 cells expressing the relevant MHC molecule were used as antigen presenting cells. Cells were also plated with PHA, 10 μg/ml, for the positive control wells, and with no peptide added for the negative control wells. Cells were discarded and the plate was washed with 0.05% Tween 10/PBS (Gibco, Life Technologies). A secondary antibody, biotinylated mouse anti-human IFN gamma monoclonal antibody (Pharmingen) was added to the wells for 3-4 hours at 1 μg/ml, then washed as before. Streptaviden-alkaline phosphatase (Pharmingen) was added for a one hour incubation, with subsequent washes as before. Lastly, BCIP-NBT buffer (Sigma) was aded for color development for 45 minutes. The plate was washed several times with deionized water and allowed to dry thoroughly. Spots were counted using a dissecting microscope (Leica, Inc.) ELIspot wells that contained a number of spots that was at least twice background and also contained greater than 20 spots per one million cells (equivalent to a ratio of 1 responder per 50,000 PBMC, above background) were considered positive, according to the criteria described by Schmechel et al., Immunol Lett 79:21-27 (2001).

A summary of the results are presented below in Table 32:

TABLE 32

| Allele | # tested | # binders | % binders | # ELIspot | % ELIspot |
|---|---|---|---|---|---|
| A2 | 25 | 13 | 52 | 6 | 24 |
| A11 | 25 | 23 | 92 | 10 | 40 |
| B7 | 25 | 21 | 84 | 11 | 44 |
| A3 | 25 | ND | ND | 16 | 64 |
| All peptides | 75 | 57 | 76 | 43 | 43 |

Fifty seven (76%) of 75 peptides tested in binding studies bound to the T2-HLA cells expressing the corresponding MHC molecule, including all of the control (published) ligands. Forty-three of 100 peptides (43%) including all of the control (published) epitopes tested in ELIspot assays stimulated gamma interferon release. EpiMatrix predicted and in vitro assays confirmed MHC-restriction by more than one HLA allele for 8 of the novel epitopes; of these epitopes, 5 were recongied in the context of MHC “supertypes” and three were promiscuous epitopes. Eighteen of the 43 confirmed epitopes (and 12 of the 32 novel epitopes) were completely conserved in more than one in 10 (10%) HIV-1 protein sequences in the Genbank database.

With regard to the A2 peptides of Table 7, thirteen of the 25 A2 peptides, including the control, (52%) selected by Conservatrix and EpiMatrix bound to T2 cells expressing HLA-A2 (T2-A2). In negative control assays none of 8 non-A2 restricted peptides stabilized the HLA-A2 MHC molecule on T2-A2 cells. ELIspot assays carried out on PBMC from 8 subjects who possessed the A2 allele using the 25 A2 (including one control) peptide. Six of the 25 A2 peptides, including the control, stimulated gamma interferon secretion from HIVB-infected subjects PBMC in vitro (24%). Two subjects did not respond to any of the selected peptides (including the control) but their cells did releae gamma-interferon. PBMC from six subjects responded to at least one A2 peptide. The average number of responses per subject, excluding subjects who did not respond to any of the peptides, was two.

With regard to the A11 peptides of Table 9, 23 of the 25 A11 peptides selected by Conservatrix and EpiMatrix bound to T2 cells expressing the A11 allele (92%), including the control peptide. In contrast, none of six A2 and B7 peptides used as negative controls bound. ELIspot assays were carried out on PBMC from six subjects who possessed the A11 allele using the 25 A11 peptides. Two subjects did not respond to any of the peptides but did respond to PHA in vitro. Ten of the A11 peptides (40%), including the control, stimulated ELIspot responses from PBMC obtained from the remaining four subjects. All but one of the peptides were binders in the T2 binding assay. The average number of responses per subject was 4.

With regard to the B7 peptides of Table 14, 21 of the 25 peptides selected by Conservatrix and EpiMatrix stabilized B7 molecules in the HLA B7-transfected T2 cell binding assay (84%), including the control peptide. None of the 8 A2 and A11 peptides used as controls stabilized B7. ELIspot assays were carried out on PBMC from three subjects who possessed the B7 allele and one subject who possessed the B8 allele using the 25 B7 peptides. Eleven of the 25 B7 peptides stimulated gamma interferon response (44%). PBMC from all four subjects responded to the peptides. The number of responses per subject ranged from 1 to 8; the average number of responses was 4.

With regard to the A3 peptides of Table 8, because functional monoclonal reagents having a reasonably low background level could not be obtained, only T cell responses to the A3 peptides were analyzed; binding assays were not performed. In ELIspot assays, 16 of the T3 peptides stimulated gamma interferon release, including the control peptide. All six subjects responding to the A3 selected peptides possessed the A3 allele. Three subjects did not respond to any A3 peptides, including the control, although these subjects did respond to PHA. The number or responses per subject when non-responders were excluded ranged from 11 to 3. The average number or responses per subject was 6.

These results demonstrate that Conservatrix and EpiMatrix permit selection of highly conserved HIV-1 T cell epitopes from among ten of millions of epitope candidates (more than 55,000 HIV-1 sequences×average 660 amino acids per sequence×10 mer overlapping frames). Representative conserved peptides for eight major HIV-1 proteins were selected and 25 peptides each for four HLA alleles (A2, A3, A11 and B7) were tested in vitro. The A2 and A3 alleles are highly prevalent worldwide. A11 is more common in Asian populations and B7 is more common in African and African American populations. 43% of epitopes selected stimulated ELIspot responses in vitro. Epitopes identified using the foregoing methods are highly conserved in isolates derived from a wide range of countries. It is possible that this analysis has uncovered regions of HIV-1 that are essential to the survival of the virus. For example, these regions may be relevant for binding to cellular receptors, to the function of certain proteins, or may be related to the three-dimensional configuration of one or the virus' proteins.

CD8+/CD4+ depletion was not performed prior to ELIspot assays; thus, some of the responses observed could possibly be due to Class 11 restriction. However, the HLA restriction for most of these epitopes was confirmed in binding studies using T2 cells expressing a single MHC molecule and generally these epitopes did no bind to T2 cell expressing MHC class I molecules for which they were predicted not to bind. Furthermore, where more than one subject responded to a peptide, the subjects were only matched for the HLA-A or HLA-B allele corresponding to the peptide selections. Since, by chance, it is extremely unlikely the responding cells were matched at more than one of their alleles, including Class II, all of the in vitro responses observed would likely be due to CD8+ restricted responses. In general, ELIspot responses to these peptides provide additional confirmatory evidence that cross-clade CTL epitopes can be identified. The results described here demonstrate that Conservatirx and EpuiMatrix can be used to identify supertype, promiscuous, dominant and subdominant CTL epitopes that can be used to stimulate a broad-based, multi-epitope, multi-allele CTL response in a prophylactic and in a therapeutic context.

The details of one or more embodiments of the invention are set forth in the accompanying description above. Although any methods and materials similar or equivalent to those described herein can be used in the practice or testing of the present invention, the preferred methods and materials have been described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

The foregoing description has been presented only for the purposes of illustration and is not intended to limit the invention to the precise form disclosed, but only to the claims appended hereto.

We claim:

1. A cross-clade HIV candidate peptide characterized by:
(i) comprising a sequence of between eight and fifty amino acids, said sequence having complete, sequential, sequence identity with a partial HIV-1 amino acid sequence that is absolutely conserved across at least 2 clades of HIV; and possessing at least one of the biological properties selected from the group consisting of:
(ii) the ability to bind a human MHC binding matrix motif for a human MHC allele;
(iii) the ability to bind human MHC HLA in the T2 in vitro peptide binding assay, as demonstrated by exhibition of greater than 1.3-fold increase in MFI upon FACS analysis; and
(vi) the ability to activate T cells from HIV positive patients in at least one in vitro assay selected from the group consisting of the ELIspot T cell assay, the ELIspot T cell restimulation assay, T cell proliferation assays, intracellular cytokine staining assays, the Brefeldin incorporation assay and tetramer staining technique.

2. A sequence according to claim 1 wherein said sequence comprises between eight and twenty-five amino acids.

3. A sequence according to claim 1 wherein said sequence comprises between eight and eleven amino acids.

4. A sequence according to claim 1 wherein said binding matrix motif is an HLA-A2, HLA-A3, HLA-A11 or HLA-B7 motif.

5. A sequence according to claim 3 wherein said binding matrix motif is an HLA-A2, HLA-A3, HLA-A11 or HLA-B7 motif.

6. A sequence according to claim 3 wherein said peptide has the ability to activate T cells from HIV positive patients in the ELIspot T cell assay.

7. A cross-clade HIV candidate peptide characterized by:
(i) comprising a sequence of between eight and ten amino acids, said sequence having complete, sequential, sequence identity with a partial HIV-1 amino acid sequence that is absolutely conserved across at least 2 clades of HIV; and possessing
(ii) the ability to bind a human MHC binding matrix motif for a human HLA allele selected from the group consisting of A2, A3, A11 and B7 alleles;
(iii) the ability to bind human MHC HLA in the T2 in vitro peptide binding assay, as demonstrated by exhibition of greater than 1.3-fold increase in MFI upon FACS analysis; and
(iv) the ability to activate T cells from HIV positive patients in at least one in vitro assay selected from the group consisting of the ELIspot T cell assay, the ELIspot T cell restimulation assay, T cell proliferation assays, intracellular cytokine staining assays, the Brefeldin incorporation assay and tetramer staining technique.

8. A polynucleotide encoding a sequence according to claim 1.

9. A polynucleotide encoding a sequence according to claim 7.

10. A vector comprising a polynucleotide according to claim 1.

11. A vector comprising a polynucleotide according to claim 9.

12. A host cell transformed with a vector according to claim 10 in operative association with an expression control sequence capable of directing replication and expression of the polynucleotide sequence in said vector.

13. A host cell transformed with a vector sequence according to claim 11 in operative association with an expression control sequence capable of directing replication and expression of the polynucleotide sequence in said vector.

14. A method of producing a cross-clade HIV peptide sequence comprising culturing a host cell according to claim 12 in a suitable culture medium and isolating said peptide sequence from said medium.

15. A method of producing a cross-clade HIV peptide sequence comprising culturing a host cell according to claim 13 in a suitable culture medium and isolating said peptide sequence from said medium.

16. A pharmaceutical composition comprising a peptide sequence according to claim 1 in admixture with a pharmaceutically acceptable excipient.

17. A pharmaceutical composition comprising a polynucleotide sequence according to claim 8 in admixture with a pharmaceutically acceptable excipient.

18. A pharmaceutical composition comprising a polynucleotide sequence according to claim 9 in admixture with a pharmaceutically acceptable excipient

19. A method for the treatment of HIV infection comprising administering to a patient a pharmaceutical composition according to claim 16 in an amount sufficient to stimulate an immune response in said patient.

20. A method according to claim 19 wherein said treatment is a prophylactic treatment.
